(19) United States
(12) Patent Application Publication
(10) Pub. No.: US 2012/0252791 A1
(43) Pub. Date: Oct. 4, 2012

(54) Heterocyclic GTP Cyclohydrolase 1 Inhibitors For the Treatment of Pain

(76) Inventor: Julian Blagg, Surrey (GB)

(21) Appl. No.: 13/496,803

(22) PCT Filed: Sep. 16, 2010
(86) PCT No.: PCT/US10/49099

§ 371 Date: Jun. 4, 2012

Related U.S. Application Data
(60) Provisional application No. 61/243,430, filed on Sep. 17, 2009.

(51) Int. Cl. A61K 31/522; A61K 31/55; A61P 29/00; C12N 5/071; C07D 473/18

(52) U.S. Cl. 514/217.06; 435/375; 544/276; 514/263.37; 540/600

(57) ABSTRACT

The present invention relates to the field of small molecule heterocyclic inhibitors of GTP cyclohydrolase (GCH-I), or a tautomer, prodrug, or pharmaceutically acceptable salt thereof. The invention also features pharmaceutical compositions of the compounds and the medical use of these compounds for the treatment or prevention of pain (e.g., inflammatory pain, nociceptive pain, functional pain, or neuropathic pain).

### CROSS-REFERENCE TO RELATED APPLICATIONS

This application claims benefit of U.S. Provisional Application No. 61/243,430, filed Sep. 17, 2009, which is hereby incorporated by reference.

### BACKGROUND OF THE INVENTION

The present invention relates to the field of small molecule heterocyclic inhibitors of GTP cyclohydrolase (GCH-1), and to the medical use of these compounds.

Tetrahydrobiopterin (BH4), which has the following structure,

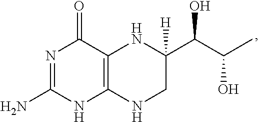

is an essential cofactor of hydroxylase enzymes that are involved in the synthesis of neurotransmitters such as serotonin, melatonin, dopamine, norepinephrine (noradrenaline), epinephrine (adrenaline), and nitric oxide (NO). GCH-1 is the enzyme that catalyzes the rate limiting step of BH4 biosynthesis, and this enzyme was found to be regulated in the dorsal root ganglion (DRG) following sciatic nerve injury (Costigan et al., BMC Neurosci 3:16, 2002). It has also been found that increased BH4 concentrations, resulting from the upregulation of GCH-1, follow axonal injury (Tegeder et al., Nature Medicine 12:1269-1277, 2006). Inhibition of this de novo BH4 synthesis in animal models resulted in reduction of neuropathic and inflammatory pain (Tegeder et al., ibid.).

Accordingly, inhibitors of GCH-1 represent beneficial therapeutics for the treatment or prevention of pain.

### SUMMARY OF THE INVENTION

In general, in a first aspect, the invention features compounds having a structure according to Formula (I),

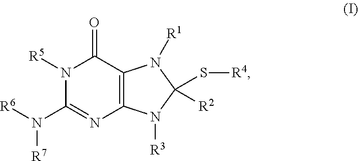

or a tautomer, prodrug, or pharmaceutically acceptable salt thereof, wherein:

R¹, R², R³, and R⁴ are each, independently, H or optionally substituted C₁₋₆ alkyl, or R¹ and R², R² and R³, or R² and R⁴ combine to form a double bond,

R⁵, R⁶, and R⁷ are each, independently, H or optionally substituted C₁₋₆ alkyl, and

wherein one and only one of R¹ and R², R² and R³, or R² and R⁴ combine to form a double bond.

In certain embodiments, when R⁵, R⁶, and R⁷ are H, R¹ and R² combine to form a double bond, and R³ is H, or when R⁵, R⁶, and R⁷ are H, R² and R³ combine to form a double bond and R¹ is H, R⁴ is not —CH₂C₆H₅, —CH₂(p-C₆H₄—CN), CH₂(p-C₆H₄—CH₃), —CH₂CH═CH₂, —CH₂C(═O)-(p-C₆H₄-OMe), —CH₂C(═O)NH-(o-C₆H₄-OEt), —CH₂C(═O)NH-(2-methoxy-5-chloro-C₆H₃), —CH₂C(═O)NH-(2-methylcyclohexyl), or —CH₂C(═O)NH-(p-C₆H₄—SO₂(azepane)).

In some embodiments, R⁵, R⁶, and R⁷ are each H.

In some embodiments, R⁶ is optionally substituted C₁₋₆ alkyl.

In some embodiments, R¹ and R² combine to form a double bond. In further embodiments, R³ is H.

In some embodiments, R² and R³ combine to form a double bond. In further embodiments, R¹ is H.

In other embodiments, R⁴ is an optionally substituted C₁₋₆ alkyl. In further embodiments, the C₁₋₆ alkyl group includes a substituent selected from aryl, heteroaryl, cycloalkyl, heterocyclyl, alkenyl, hydroxyl, C₁₋₃ alkoxy, amino, or C₁₋₆ alkylamino, and the aryl or heteroaryl is optionally substituted (e.g., by C₁₋₄ alkyl, halogen, or nitrile).

In further embodiments, the compound of Formula (I) may have a structure according to

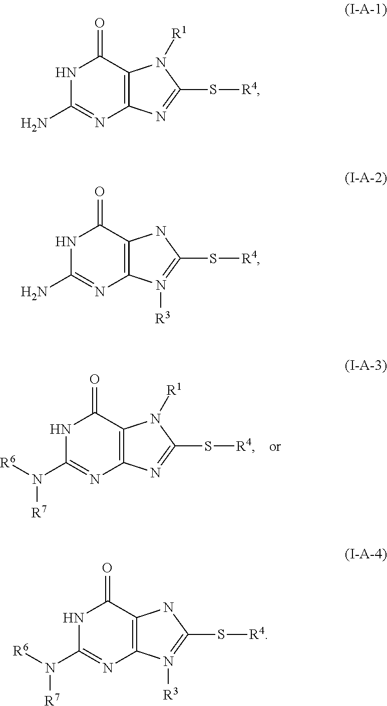

where each R¹, R³, R⁴, R⁶, and R⁷ is as defined for Formula (I).

In yet other embodiments, the compound of Formula (I) is selected from:

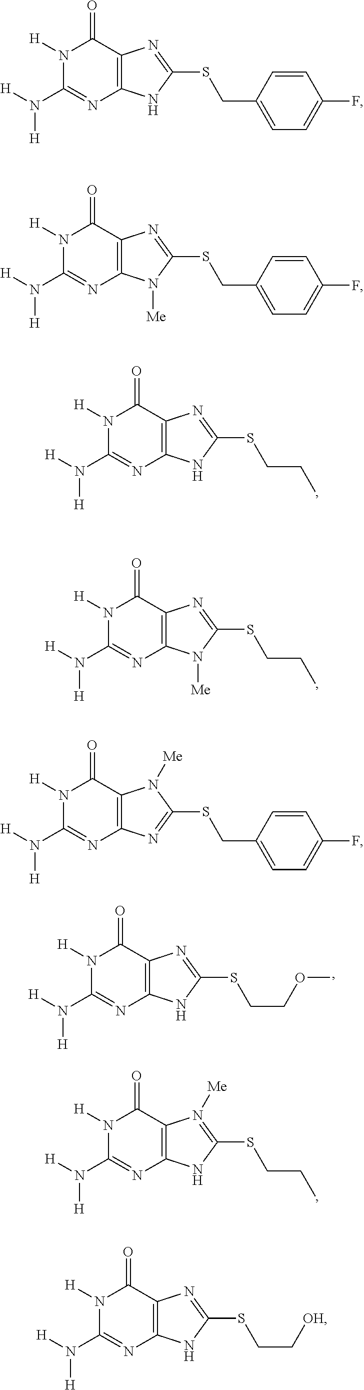

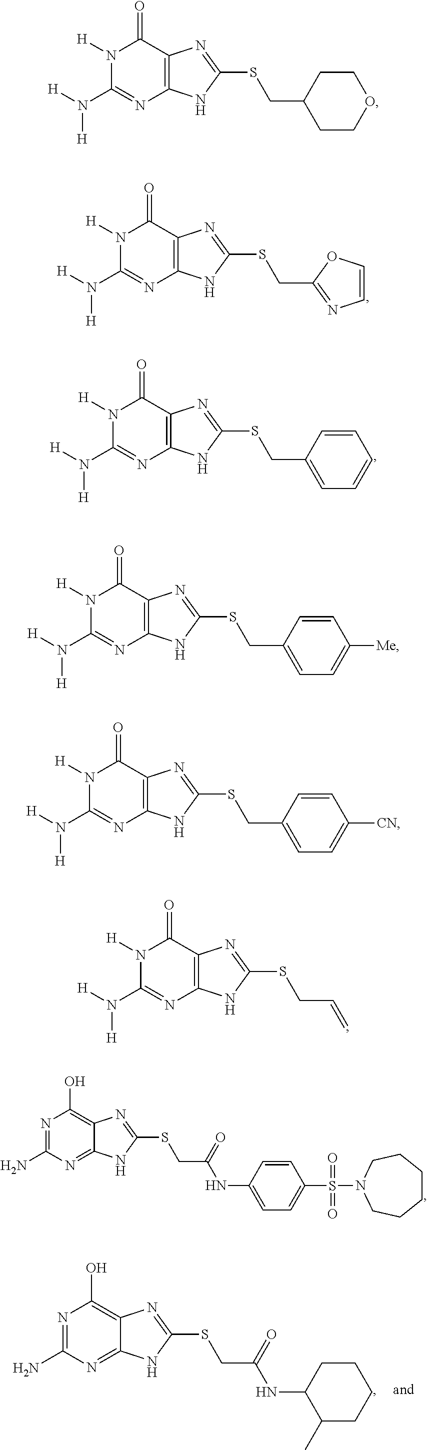

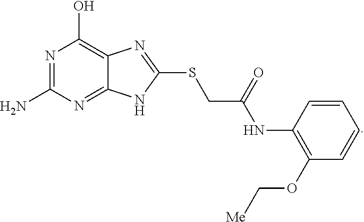

Alternatively, R² and R⁴ may combine to form a double bond. In certain other embodiments, the compound of Formula (I) may have a structure according to

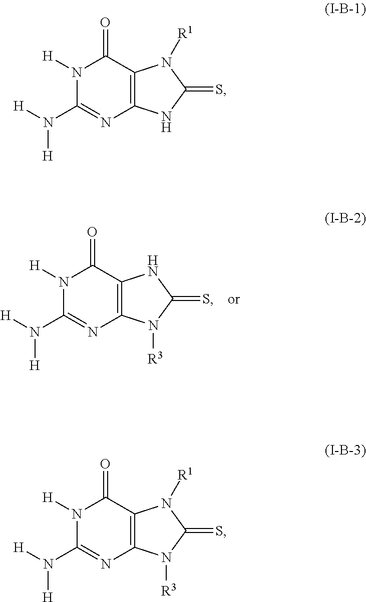

where each R¹ and R³ is as defined for Formula (I).

In certain embodiments, the compound is selected from the group consisting of:

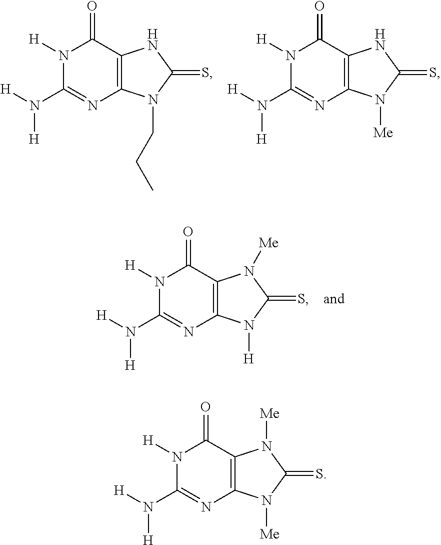

In a further embodiment, any compound according to Formula (I) may be an inhibitor of GTP cyclohydrolase (GCH-1).

In a second aspect, the invention features further compounds having a structure according to Formula (II-A),

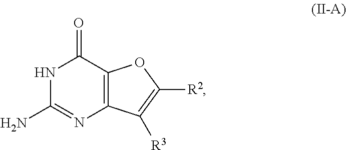

or a tautomer, prodrug, or pharmaceutically acceptable salt thereof,

or having a structure according to Formula (II-B),

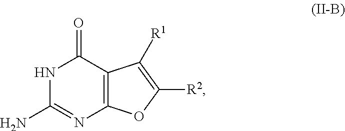

or a tautomer, prodrug, or pharmaceutically acceptable salt thereof, wherein R¹, R², and R³ are each, independently, H or optionally substituted C₁₋₆ alkyl.

The C₁₋₆ alkyl may include a substituent selected from aryl, heteroaryl, cycloalkyl, heterocyclyl, hydroxyl, C₁₋₃ alkoxy, amino, or C₁₋₆alkylamino, and the aryl or heteroaryl may be optionally substituted (e.g., by C₁₋₄ alkyl, halogen, or nitrile).

R² may be H.

In further embodiments, the compound is selected from the group consisting of:

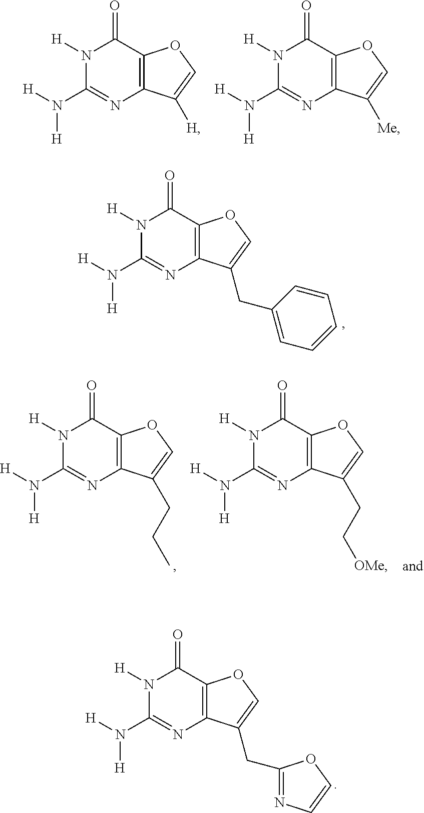

For any of the above embodiments, the compound of Formula (II-A) or (II-B) may be an inhibitor of GTP cyclohydrolase (GCH-1).

In a third aspect, the invention relates to compounds having a structure according to Formula (III):

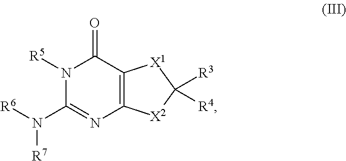

or a tautomer, prodrug, or pharmaceutically acceptable salt thereof, wherein

X¹ is O or NR¹;

X² is O or NR²;

R¹ and R² are each, independently, selected from H, or optionally substituted C₁₋₆ alkyl;

R³ is H, halogen (e.g., F, Cl, Br, or I), or or NR⁸R⁹, or R³ combines with R⁴ to form an oxo group; and

R⁴ combines with R¹ or R² to form a C═N bond or R⁴ combines with R³ to form an oxo group;

R⁵, R⁶, R⁷, R⁸, and R⁹ are each, independently, H or optionally substituted C₁₋₆ alkyl; and

when R⁵, R⁶, and R⁷ are H, X′ is NR¹, R¹ and R⁴ combine to form a C═N double bond, and X² is NH, R³ is not H or NH₂, and

when R⁵, R⁶, and R⁷ are H, X¹ is NH, R³ combines with R⁴ to form an oxo group, and X² is NR², R² is not H.

In some embodiments, R⁵, R⁶, R⁷, R⁸, and R⁹ are each H.

In certain embodiments, the C₁₋₆ alkyl includes a substituent selected from aryl, heteroaryl, cycloalkyl, heterocyclyl, hydroxyl, C₁₋₃ alkoxy, amino, or C₁₋₆alkylamino, wherein the aryl or heteroaryl is optionally substituted (e.g., by C₁₋₄ alkyl, halogen, or nitrile).

In other embodiments, the compound has the following structure:

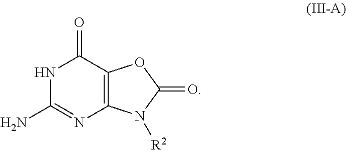

In further embodiments, the compound is selected from the group consisting of:

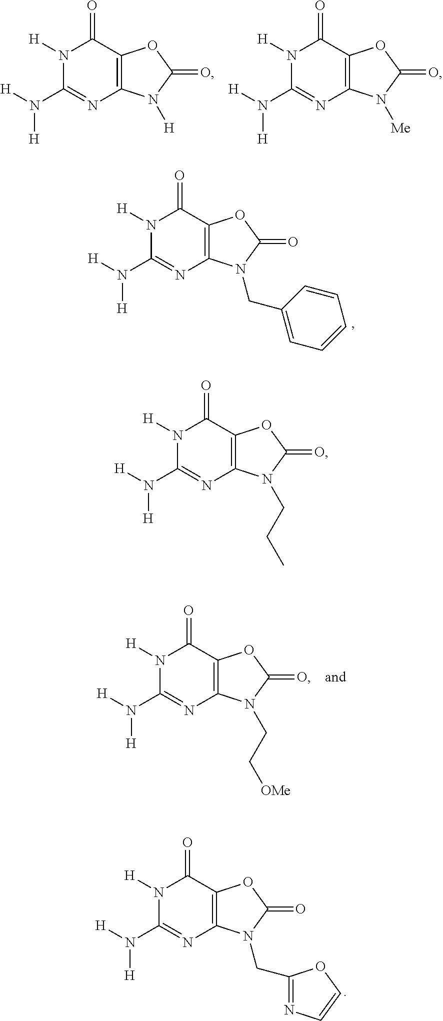

In other embodiments, X¹ is NR¹, X² is NR², R¹ and R² are each, independently, H or optionally substituted C₁₋₆ alkyl, and R³ combines with R⁴ to form an oxo group. In further embodiments, the compound is

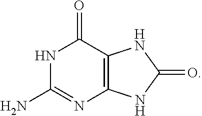

In still other embodiments, the compound has a structure according to

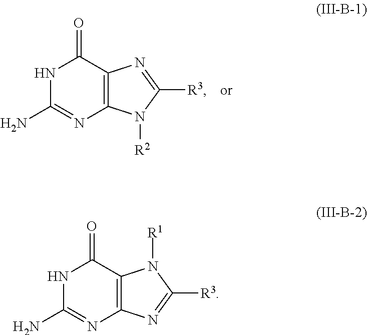

In some embodiments, R³ is H, Cl, or Br. In other embodiments, R² is optionally substituted C₁₋₆, alkyl. In further embodiments, the C₁₋₆ alkyl includes a substituent selected from aryl, heteroaryl, cycloalkyl, heterocyclyl, hydroxyl, C₁₋₃ alkoxy, amino, or C₁₋₆alkylamino, wherein the aryl or heteroaryl is optionally substituted (e.g., by C₁₋₄ alkyl, halogen, or nitrile).

In yet further embodiments, the compound of Formula (III-B-1) or (III-B-2) is selected from the group consisting of:

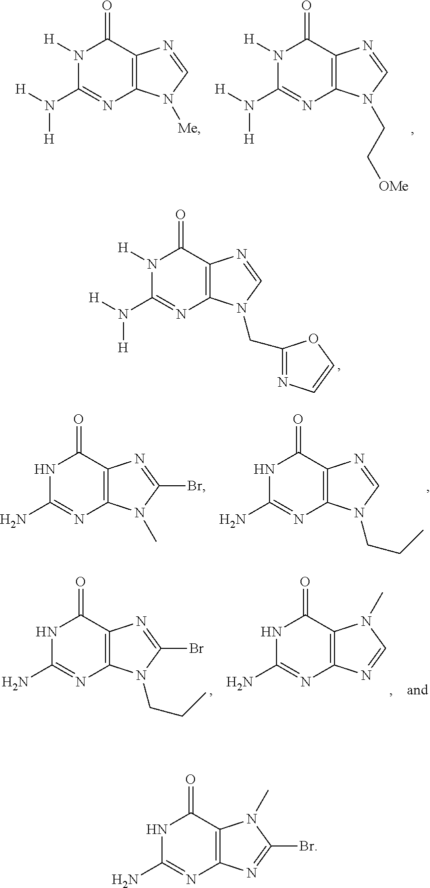

Alternatively, the compound may have a structure according to the following formula:

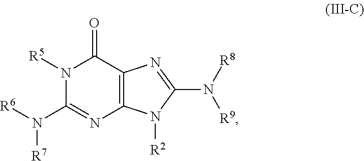

wherein R² is H or optionally substituted C₁₋₆ alkyl.

In some embodiments, R⁵, R⁶, R⁷, R⁸, and R⁹ are each H. In certain embodiments, R² is H. In other embodiments, R² is optionally substituted C₁₋₆ alkyl.

In further embodiments, the C₁₋₆ alkyl includes a substituent selected from aryl, heteroaryl, cycloalkyl, heterocyclyl, hydroxyl, C₁₋₃ alkoxy, amino, or C₁₋₆alkylamino, wherein the aryl or heteroaryl is optionally substituted (e.g., by C₁₋₄ alkyl, halogen, or nitrile). In still other embodiments, the compound can be

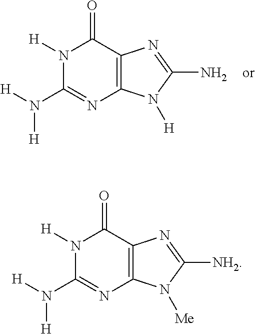

In another embodiment, the compound of Formula (III) (e.g., a compound of Formula (III-A), (III-B-1), (III-B-2), or (III-C)) may be an inhibitor of GTP cyclohydrolase (GCH-1).

In a fourth aspect, the invention relates to compounds having a structure according to

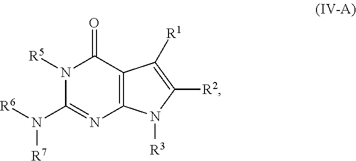

or a tautomer, prodrug, or pharmaceutically acceptable salt thereof, or according to

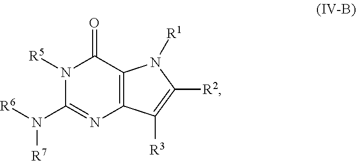

or a tautomer, prodrug, or pharmaceutically acceptable salt thereof, wherein R¹, R², R³, R⁵, R⁶, and R⁷ are each, independently, H or optionally substituted C₁₋₆ alkyl.

In some embodiments, R⁵, R⁶, and R⁷ are each H.

In other embodiments, R¹ and R³ are both II.

In certain embodiments, the C₁₋₆ alkyl includes a substituent selected from aryl, heteroaryl, cycloalkyl, heterocyclyl, hydroxyl, C₁₋₃ alkoxy, amino, or C₁₋₆alkylamino, and wherein said aryl or heteroaryl is optionally substituted (e.g., by C₁₋₄ alkyl, halogen, or nitrile). In other embodiments, R² is H. In still other embodiments, the compound is selected from the group consisting of:

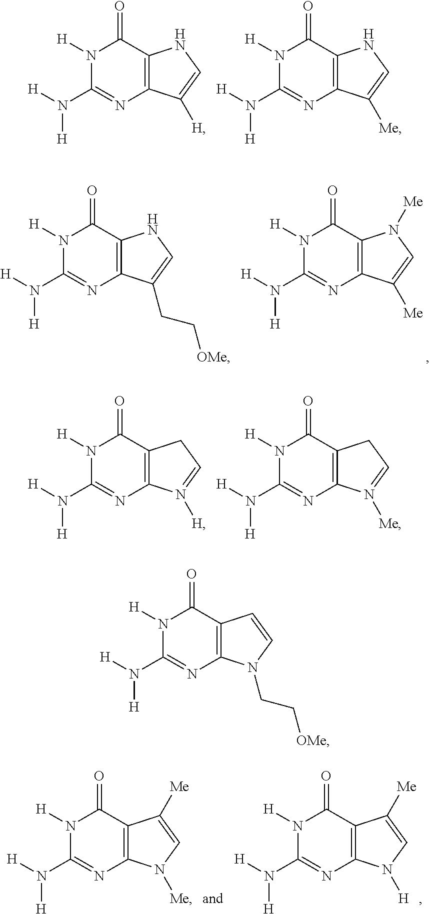

In a further embodiment, the compound of Formula (IV) (e.g., a compound of Formula (IV-A) or (IV-B)) may be an inhibitor of GTP cyclohydrolase (GCH-1).

In a fifth aspect, the invention features compounds having a structure according to

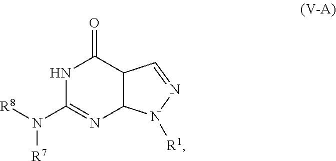

or a tautomer, prodrug, or pharmaceutically acceptable salt thereof, or

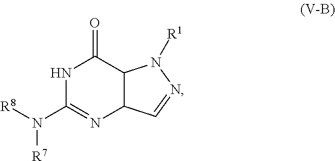

or a tautomer, prodrug, or pharmaceutically acceptable salt thereof, wherein R¹ is H or optionally substituted C₁₋₆ alkyl, and R⁶ and R⁷ are each, independently, H or optionally substituted C₁₋₆ alkyl. In some embodiments, R⁶ and R⁷ are both H. In some embodiments, the C₁₋₆ alkyl includes a substituent selected from aryl, heteroaryl, cycloalkyl, heterocyclyl, hydroxyl, C₁₋₃ alkoxy, amino, or C₁₋₆alkylamino, wherein the aryl or heteroaryl is optionally substituted (e.g., by C₁₋₄ alkyl, halogen, or nitrile). In certain embodiments, R¹ is H. In other embodiments, the compound is

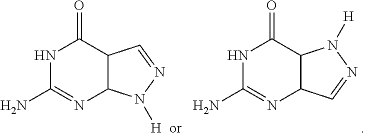

In another embodiment, the compound of Formula (V) (e.g., a compound of Formula (V-A) or (V-B)) may be an inhibitor of GTP cyclohydrolase (GCH-1).

In a sixth aspect, the invention features compounds having a structure according to Formula (VI),

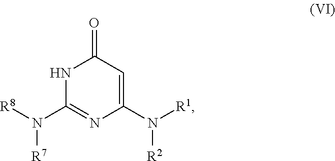

or a tautomer, prodrug, or pharmaceutically acceptable salt thereof, wherein each of R¹, R², R⁶, and R⁷ is, H or optionally substituted C₁₋₆ alkyl. In some embodiments, R⁶ and R⁷ are both H. In certain embodiments, the compound of Formula (VI) is

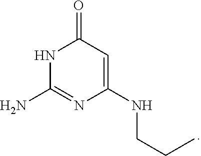

In a related aspect, the invention relates to a pharmaceutical composition that includes any of the compounds (e.g., in an effective amount) described herein (e.g., a compound of Formula (I), (II), (III), (IV), (V), or (VI), or any of Compounds (1)-(56)), or a tautomer, prodrug, or pharmaceutically acceptable salt thereof, and a pharmaceutically acceptable excipient.

The pharmaceutical composition may include an effective amount of the compound (e.g., a compound of Formula (I), (II), (III), (IV), (V), or (VI), or any of Compounds (1)-(56)), or a tautomer, prodrug, or pharmaceutically acceptable salt thereof.

In another related aspect, the invention relates to a method of treating, reducing, or preventing a condition in a mammal, wherein said method includes the administration of any of the compounds described herein (e.g., a compound of Formula (I), (II), (III), (IV), (V), or (VI), or any of Compounds (1)-(56)), or a tautomer, prodrug, or pharmaceutically acceptable salt thereof, or a pharmaceutical composition thereof, to the mammal in a dosage sufficient to inhibit GCH-1. In one embodiment, the condition is pain. The pain may be neuropathic, inflammatory, nociceptive, or functional pain. Further, the pain may be chronic or acute.

Finally, the invention relates to a method of inhibiting GCH-1 in a cell, involving contacting a cell with any of the compounds described herein (e.g., a compound of Formula (I), (II), (III), (IV), (V), or (VI), or any of Compounds (1)-(56)), or a tautomer, prodrug, or pharmaceutically acceptable salt thereof.

The term “C_{x-y} alkaryl,” as used herein, represents a chemical substituent of formula —RR′, where R is an alkylene group of x to y carbons and R′ is an aryl group as defined herein. Similarly, by the term “C_{x-y} alkheteroaryl” is meant a chemical substituent of formula —RR″, where R is an alkylene group of x to y carbons and R″ is a heteroaryl group as defined herein. Other groups preceded by the prefix “alk-” are defined in the same manner. Exemplary unsubstituted alkaryl groups are of from 7 to 16 carbons.

The term “alkcycloalkyl” represents a cycloalkyl group attached to the parent molecular group through an alkylene group.

The terms “alkenyl” or “C₂₋₆ alkenyl,” as used herein, represent monovalent straight or branched chain groups of, unless otherwise specified, from 2 to 6 carbons containing one or more carbon-carbon double bonds and is exemplified by ethenyl, 1-propenyl, 2-propenyl, 2-methyl-1-propenyl, 1-butenyl, 2-butenyl, and the like. A substituted C₂₋₆ alkenyl may have, for example, 1, 2, 3, 4, 5, or 6 substituents located at any position.

The term “alkheterocyclyl” represents a heterocyclic group attached to the parent molecular group through an alkylene group. Exemplary unsubstituted alkheterocyclyl groups are of from 2 to 14 carbons.

The term “alkoxy” represents a chemical substituent of formula —OR, where R is an optionally substituted alkyl group of 1 to 6 carbons, unless otherwise specified (e.g., “C₁₋₃alkoxy” refers to alkoxy groups including a C₁₋₃alkyl group), where the optionally substituted alkyl may be branched, linear, or cyclic. The C₁₋₆ alkyl may be substituted or unsubstituted. A substituted C₁₋₆ alkyl can have, for example, 1, 2, 3, 4, 5, or 6 substituents located at any position. Exemplary alkoxy groups include, but are not limited to, methoxy, ethoxy, propoxy, isopropoxy, tert-butoxy, and the like.

The terms “alkyl” and the prefix “alk-,” as used herein, are inclusive of both straight chain and branched chain saturated groups of from 1 to 6 carbons, unless otherwise specified (e.g., “C₁₋₄alkyl” refers to alkyl groups having 1-4 carbons). Alkyl groups are exemplified by methyl, ethyl, n- and iso-propyl, n-, sec-, iso- and tert-butyl, neopentyl, and the like, and may be optionally substituted. Exemplary substituted alkyl groups include, but are not limited to, optionally substituted C₁₋₄ alkaryl groups.

The term “C₁₋₆alkylamino,” as used herein, represents an alkyl group, as defined herein, substituted by an amino group.

The term “alkylene,” as used herein, represents a saturated divalent hydrocarbon group derived from a straight or branched chain saturated hydrocarbon by the removal of two hydrogen atoms, and is exemplified by methylene (—CH₂—), ethylene (—CH₂CH₂—), isopropylene, and the like.

By “amino” is meant a group having a structure —NR′R″, where each R′ and R″ is selected, independently, from H, optionally substituted C₁₋₆ alkyl, optionally substituted cycloalkyl, optionally substituted heterocyclyl, optionally substituted aryl, optionally substituted heteroaryl, or R′ and R″ combine to form an optionally substituted heterocyclyl. When R′ is not H or R″ is not H, R′ and R″ may be unsubstituted or substituted with, for example, 1, 2, 3, 4, 5, or 6 substituents.

By “aryl” is meant is an optionally substituted C₆-C₁₀ cyclic group with [4n+2] π electrons in conjugation and where n is 1, 2, or 3. Non-limiting examples of aryls include heteroaryls and, for example, benzene and naphthalene. Aryls also include bi- and tri-cyclic ring systems in which a non-aromatic saturated or partially unsaturated carbocyclic ring (e.g., a cycloalkyl or cycloalkenyl) is fused to an aromatic ring such as benzene or napthalene. Exemplary aryls fused to a non-aromatic ring include indanyl and tetrahydronaphthyl. Any aryls as defined herein may be unsubstituted or substituted. A substituted aryl may be optionally substituted with, for example, 1, 2, 3, 4, 5, or 6 substituents located at any position of the ring.

By “cycloalkyl” is meant an optionally substituted, saturated or partially unsaturated 3- to 10-membered monocyclic or polycyclic (e.g., bicyclic, or tricyclic) hydrocarbon ring system. Where a cycloalkyl is polycyclic, the constituent cycloalkyl rings may be fused together, form a spirocyclic structure, or the polycyclic cycloalkyl may be a bridged cycloalkyl (e.g., adamantyl or norbonanyl). Exemplary cycloalkyls induce cyclopropyl, cyclobutyl, cyclopentyl, cyclohexyl, and cycloheptyl. Cycloalkyls may be unsubstituted or substituted. A substituted cycloalkyl can have, for example, 1, 2, 3, 4, 5, or 6 substituents.

The term “cycloalkyl,” as used herein represents a monovalent saturated or unsaturated non-aromatic cyclic hydrocarbon group of from three to eight carbons, unless otherwise specified, and is exemplified by cyclopropyl, cyclobutyl, cyclopentyl, cyclohexyl, cycloheptyl, bicyclo[2.2.1]heptyl and the like. The cycloalkyl groups of this invention can be optionally substituted

The term an “effective amount” of a compound (e.g., any of Compounds (1)-(56) or any of the compounds according to Formulas (I)-(VI)), as used herein, is that amount sufficient to effect beneficial or desired results, such as clinical results, and, as such, an “effective amount” depends upon the context in which it is being applied. For example, in the context of administering an agent that inhibits GCH-1, an effective amount of an agent is, for example, an amount sufficient to achieve a reduction in GCH-1 activity as compared to the response obtained without administration of the agent and thereby prevents, reduces, or eliminates the sensation of pain. The effective amount of active compound(s) used to practice the present invention for therapeutic treatment of pain also varies depending upon the manner of administration, the age, and body weight, of the subject as well as the underlying pathology that is causing the pain. Ultimately, the attending physician or veterinarian will decide the appropriate amount and dosage regimen.

By “halogen” or “halo” is meant fluorine (—F), chlorine (—Cl), bromine (—Br), or iodine (—I).

The term “heteroaryl,” as used herein, represents that subset of heterocycles, as defined herein, which are aromatic: i.e., they contain 4n+2 pi electrons within the mono- or multicyclic ring system. Exemplary heteroaryls include, but are not limited to, furan, thiophene, pyrrole, thiadiazole (e.g., 1,2,3-thiadiazole or 1,2,4-thiadiazole), oxadiazole (e.g., 1,2,3-oxadiazole or 1,2,5-oxadiazole), oxazole, benzoxazole, isoxazole, isothiazole, pyrazole, thiazole, benzthiazole, triazole (e.g., 1,2,4-triazole or 1,2,3-triazolc), benzotriazole, pyridines, pyrimidines, pyrazines, quinoline, isoquinoline, purine, pyrazine, pteridine, triazine (e.g, 1,2,3-triazine, 1,2,4-triazine, or 1,3,5-triazine)indoles, 1,2,4,5-tetrazine, benzo[b]thiophene, benzo[c]thiophene, benzofuran, isobenzofuran, and benzimidazole. Heteroaryls may be unsubstituted or substituted. Substituted heteroaryls can have, for example, 1, 2, 3, 4, 5, or 6 substitutents.

The terms “heterocycle” or “heterocyclyl,” as used interchangeably herein represent a 5-, 6- or 7-membered ring, unless otherwise specified, containing one, two, three, or four heteroatoms independently selected from the group consisting of nitrogen, oxygen and sulfur. The 5-membered ring has zero to two double bonds and the 6- and 7-membered rings have zero to three double bonds. The term “heterocyclyl” also represents a heterocyclic compound having a bridged multicyclic structure in which one or more carbons and/or heteroatoms bridges two non-adjacent members of a monocyclic ring, e.g., a quinuclidinyl group. The term “heterocycle” includes bicyclic, tricyclic and tetracyclic groups in which any of the above heterocyclic rings is fused to one, two, or three rings, e.g., an aryl ring, a cyclohexane ring, a cyclohexene ring, a cyclopentane ring, a cyclopentene ring and another monocyclic heterocyclic ring, such as indolyl, quinolyl, isoquinolyl, tetrahydroquinolyl, benzofuryl, benzothienyl and the like. Examples of fused heterocycles include tropanes and 1,2,3,5,8,8a-hexahydroindolizine. Heterocyclics include pyrrolyl, pyrrolinyl, pyrrolidinyl, pyrazolyl, pyrazolinyl, pyrazolidinyl, imidazolyl, imidazolinyl, imidazolidinyl, pyridyl, piperidinyl, homopiperidinyl, pyrazinyl, piperazinyl, pyrimidinyl, pyridazinyl, oxazolyl, oxazolidinyl, isoxazolyl, isoxazolidinyl, morpholinyl, thiomorpholinyl, thiazolyl, thiazolidinyl, isothiazolyl, isothiazolidinyl, indolyl, quinolinyl, isoquinolinyl, benzimidazolyl, benzothiazolyl, benzoxazolyl, furyl, thienyl, thiazolidinyl, isothiazolyl, isoindazoyl, triazolyl, tetrazolyl, oxadiazolyl, uricyl, thiadiazolyl, pyrimidyl, tetrahydrofuranyl, dihydrofuranyl, tetrahydrothienyl, dihydrothienyl, dihydroindolyl, tetrahydroquinolyl, tetrahydroisoquinolyl, pyranyl, dihydropyranyl, dithiazolyl, benzofuranyl, benzothienyl and the like. Any of the heterocycle groups mentioned herein may be optionally substituted with one, two, three, four or five substituents

The term “hydroxyl,” as used herein, represents an —OH group.

The term “nitrile,” as used herein, represents a —CN group.

By “pain” is meant all types of pain including inflammatory pain, nociceptive pain, functional pain, and neuropathic pain (peripheral and central), whether acute or chronic. Exemplary, non-limiting types of pain that can be treated according to the methods described herein include musculo-skeletal pain (after trauma, infections, and exercise), neuropathic pain caused by spinal cord injury, tumors, compression, inflammation, dental pain, episiotomy pain, deep and visceral pain (e.g., heart pain, bladder pain, or pelvic organ pain), muscle pain, eye pain, orofacial pain (e.g., odontalgia, trigeminal neuralgia, glossopharyngeal neuralgia), abdominal pain, gynecological pain (e.g., dysmenorrhea and labor pain), pain associated with nerve and root damage due to trauma, compression, inflammation, toxic chemicals, metabolic disorders, hereditary conditions, infections, vasculitis and autoimmune diseases, central nervous system pain, such as pain due to spinal cord or brain stem damage, cerebrovascular accidents, tumors, infections, demyelinating diseases including multiple sclerosis, low back pain, sciatica, and post-operative pain. Pain can also be associated with conditions that include, for example, soft tissue, joint, bone inflammation and/or damage (e.g., acute trauma, osteoarthritis, or rheumatoid arthritis), myofascial pain syndromes (fibromylagia), headaches (including cluster headache, migraine, and tension type headache), myocardial infarction, angina, ischemic cardiovascular disease, post-stroke pain, sickle cell anemia, peripheral vascular occlusive disease, cancer, inflammatory conditions of the skin or joints, diabetic neuropathy, and acute tissue damage from surgery or traumatic injury (e.g., burns, lacerations, or fractures).

The term “pharmaceutical composition,” as used herein, represents a composition containing a compound (e.g., an effective amount of the compound) described herein (e.g., any of Compounds (1)-(56) or any of the compounds according to Formulas (I)-(VI)), formulated with a pharmaceutically acceptable excipient, and typically manufactured or sold with the approval of a governmental regulatory agency as part of a therapeutic regimen for the treatment of disease in a mammal. Pharmaceutical compositions can be formulated, for example, for oral administration in unit dosage form (e.g., a tablet, capsule, caplet, gelcap, or syrup); for topical administration (e.g., as a cream, gel, lotion, or ointment); for intravenous administration (e.g., as a sterile solution free of particulate emboli and in a solvent system suitable for intravenous use); or in any other formulation described herein.

A “pharmaceutically acceptable excipient,” as used herein, refers any ingredient other than the compounds described herein (for example, a vehicle capable of suspending or dissolving the active compound) and having the properties of being nontoxic and non-inflammatory in a patient. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspensing or dispersing agents, sweeteners, or waters of hydration. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, stearic acid, sucrose, talc, titanium dioxide, vitamin A, vitamin E, vitamin C, and xylitol.

The term “pharmaceutically acceptable prodrugs” as used herein, represents those prodrugs of the compounds of the present invention which are, within the scope of sound medical judgment, suitable for use in contact with the tissues of humans and animals with undue toxicity, irritation, allergic response, and the like, commensurate with a reasonable benefit/risk ratio, and effective for their intended use, as well as the zwitterionic forms, where possible, of the compounds of the invention.

The term “pharmaceutically acceptable salt,” as used herein, represents those salts which are, within the scope of sound medical judgment, suitable for use in contact with the tissues of humans and animals without undue toxicity, irritation, allergic response and the like and are commensurate with a reasonable benefit/risk ratio. Pharmaceutically acceptable salts are well known in the art. For example, pharmaceutically acceptable salts are described in: Berge et al., J. Pharmaceutical Sciences 66:1-19, 1977 and in Pharmaceutical Salts: Properties, Selection, and Use, (Eds. P. H. Stahl and C. G. Wermuth), Wiley-VCH, 2008. The salts can be prepared in situ during the final isolation and purification of the compounds of the invention or separately by reacting the free base group with a suitable organic acid. Representative acid addition salts include acetate, adipate, alginate, ascorbate, aspartate, benzenesulfonate, benzoate, bisulfate, borate, butyrate, camphorate, camphersulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undccanoate, valerate salts and the like. Representative alkali or alkaline earth metal salts include sodium, lithium, potassium, calcium, magnesium and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine and the like.

The terms “pharmaceutically acceptable solvate” or “solvate,” as used herein, means a compound of the invention wherein molecules of a suitable solvent are incorporated in the crystal lattice. A suitable solvent is physiologically tolerable at the dosage administered. For example, solvates may be prepared by crystallization, recrystallization, or precipitation from a solution that includes organic solvents, water, or a mixture thereof. Examples of suitable solvents are ethanol, water (for example, mono-, di-, and tri-hydrates), N-methylpyrrolidinone (NMP), dimethyl sulfoxide (DMSO), N,N′-dimethylformamide (DMF), N,N′-dimethylacetamide (DMAC), 1,3-dimethyl-2-imidazolidinone (DMEU), 1,3-dimethyl-3,4,5,6-tetrahydro-2-(1H)-pyrimidinone (DMPU), acetonitrile (ACN), propylene glycol, ethyl acetate, benzyl alcohol, 2-pyrrolidone, benzyl benzoate, and the like. When water is the solvent, the solvate is referred to as a “hydrate.”

The term “prevent,” as used herein, refers to prophylactic treatment or treatment that prevents one or more symptoms or conditions of a disease, disorder, or conditions described herein such as pain (e.g., neuropathic or inflammatory pain). Preventative treatment can be initiated, for example, prior to (“pre-exposure prophylaxis”) or following (“post-exposure prophylaxis”) an event that precedes the onset of the disease, disorder, or conditions. Preventive treatment that includes administration of a compound of the invention, or a pharmaceutical composition thereof, can be acute, short-term, or chronic. The doses administered may be varied during the course of preventative treatment.

The term “prodrug,” as used herein, represents compounds which are rapidly transformed in vivo to the parent compound of the above formula (e.g., any of Compounds (1)-(56) or any of the compounds according to Formulas (I)-(VI)), for example, by hydrolysis in blood. Prodrugs of the compounds of the invention may be conventional esters. Some common esters which have been utilized as prodrugs are phenyl esters, aliphatic (C₇-C₈ or C₈-C₂₄) esters, cholesterol esters, acyloxymethyl esters, carbamates, and amino acid esters. For example, a compound of the invention that contains an OH group may be acylated at this position in its prodrug form. A thorough discussion is provided in T. Higuchi and V. Stella, Pro-drugs as Novel Delivery Systems, Vol. 14 of the A.C.S. Symposium Series, Edward B. Roche, ed., Bioreversible Carriers in Drug Design, American Pharmaceutical Association and Pergamon Press, 1987, and Judkins et al., Synthetic Communications 26(23):4351-4367, 1996, each of which is incorporated herein by reference. Preferably, prodrugs of the compounds of the present invention are pharmaceutically acceptable.

The term “thioether,” as used herein, refers to a C—SR group, where R is an unsubstituted alkyl or a substituted alkyl (e.g., an alkaryl group that may be further substituted) as described herein.

The term “thiol,” as used herein, refers to the —SH group.

The term “thiooxo,” as used herein, refers to a C═S group, where a carbon atom is double-bonded to sulfur.

As used herein, and as well understood in the art, “treatment” is an approach for obtaining beneficial or desired results, such as clinical results. Beneficial or desired results can include, but are not limited to, alleviation or amelioration of one or more symptoms or conditions; diminishment of extent of disease, disorder, or condition; stabilized (i.e. not worsening) state of disease, disorder, or condition; preventing spread of disease, disorder, or condition; delay or slowing the progress of the disease, disorder, or condition; amelioration or palliation of the disease, disorder, or condition; and remission (whether partial or total), whether detectable or undetectable. “Treatment” can also mean prolonging survival as compared to expected survival if not receiving treatment. “Palliating” a disease, disorder, or condition means that the extent and/or undesirable clinical manifestations of the disease, disorder, or condition are lessened and/or time course of the progression is slowed or lengthened, as compared to the extent or time course in the absence of treatment. By “treating, reducing, or preventing pain” is meant preventing, reducing, or eliminating the sensation of pain in a subject before, during, or after it has occurred. As compared with an equivalent untreated control, such reduction or degree of prevention is at least 5%, 10%, 20%, 40%, 50%, 60%, 80%, 90%, 95%, or 100% as measured by any standard technique known in the art. To treat pain, according to the methods of this invention, the treatment does not necessarily provide therapy for the underlying pathology that is causing the painful sensation. Treatment of pain can be purely symptomatic.

Where a group is substituted, the group may be substituted with 1, 2, 3, 4, 5, or 6 substituents. Optional substituents include, but are not limited to: C₁₋₆ alkyl, C₂₋₆ alkenyl, C₂₋₆ alkynyl, cycloalkyl, cycloalkenyl, heterocyclyl, aryl, heteroaryl, halogen; azido(—N₃), nitro (—NO₂), cyano (—CN), acyloxy(—OC(═O)R′), acyl (—C(═O)R′), alkoxy (—OR′), amido (—NR′C(═O)R″ or —C(═O)NRR′), amino (—NRR′), carboxylic acid (—CO₂H), carboxylic ester (—CO₂R′), carbamoyl (—OC(═O)NR′R″ or —NRC(═O)OR′), hydroxy (—OH), isocyano (—NC), sulfonate (—S(═O)₂OR), sulfonamide (—S(═O)₂NRR′ or —NRS(═O)₂R′), or sulfonyl (—S(═O)₂R), where each R or R′ is selected, independently, from H, C₁₋₆ alkyl, C₂₋₆ alkenyl, C₂₋₆ alkynyl, cycloalkyl, heterocyclyl, aryl, or heteroaryl. A substituted group may have, for example, 1, 2, 3, 4, 5, 6, 7, 8, or 9 substituents. In some embodiments, each hydrogen in a group may be replaced by a substituent group (e.g., perhaloalkyl groups such as —CF₃ or —CF₂CF₃ or perhaloaryls such as —C₆F₅). In other embodiments, a substituent group may itself be further substituted by replacing a hydrogen of said substituent group with another substituent group such as those described herein. Substituents may be further substituted with, for example, 1, 2, 3, 4, 5, or 6 substituents as defined herein. For example, a lower C₁₋₆ alkyl or an aryl substituent group (e.g., heteroaryl, phenyl, or naphthyl) may be further substituted with 1, 2, 3, 4, 5, or 6 substituents as described herein.

Asymmetric or chiral centers may exist in any of the compounds of the present invention. The present invention contemplates the various stereoisomers and mixtures thereof. Individual stereoisomers of compounds of the present invention are prepared synthetically from commercially available starting materials that contain asymmetric or chiral centers or by preparation of mixtures of enantiomeric compounds followed by resolution well-known to those of ordinary skill in the art. These methods of resolution are exemplified by (1) attachment of a racemic mixture of enantiomers, designated (+/−), to a chiral auxiliary, separation of the resulting diastereomers by recrystallization or chromatography and liberation of the optically pure product from the auxiliary or (2) direct separation of the mixture of optical enantiomers on chiral chromatographic columns. Alternatively, chiral compounds can be prepared by an asymmetric synthesis that favors the preparation of one enantiomer over the other. Alternatively a chiral pool synthesis (starting with an enantiomerically pure building block) can be used wherein the chiral group or center is retained in the intermediate or final product. Enantiomers are designated herein by the symbols “R,” or “S,” depending on the configuration of substituents around the chiral atom. Alternatively, enantiomers are designated as (+) or (−) depending on whether a solution of the enantiomer rotates the plane of polarized light clockwise or counterclockwise, respectively. In other cases, diastereomeric isomers such as cis and trans isomers may be separated by column chromatography, chiral chromatography, or recrystallization. In some cases, derivatization can improve the separation of these mixtures.

Geometric isomers may also exist in the compounds of the present invention. The present invention contemplates the various geometric isomers and mixtures thereof resulting from the arrangement of substituents around a carbon-carbon double bond and designates such isomers as of the Z or E configuration. It is also recognized that for structures in which tautomeric forms are possible, the description of one tautomeric form is equivalent to the description of both, unless otherwise specified. For example, the invention includes the following exemplary tautomers of the respective Formulas (I)-(VI), or a prodrug or pharmaceutically acceptable salt thereof, where each R¹-R⁴, R⁶-R⁹, X¹, and X² in the tautomeric form has the same meaning as in the corresponding formula.

| Formula | Tautomer |
|---|---|
| 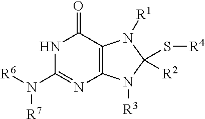 | 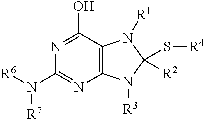 |
| (I) | |
| 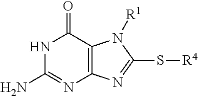 | 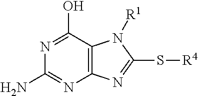 |
| (IA-1) | |
| 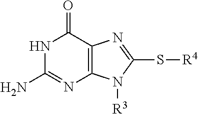 | 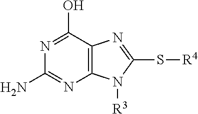 |
| (I-A-2) | |
| 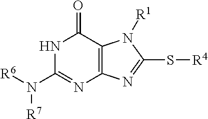 | 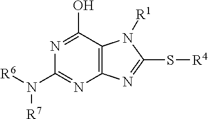 |
| (I-A-3) | |
| 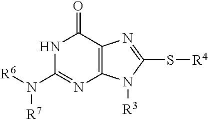 | 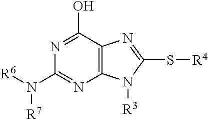 |
| (I-A-4) | |
| 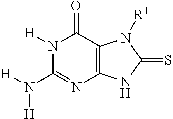 | 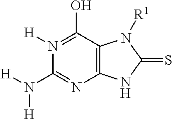 |
| (I-B-1) | |
| 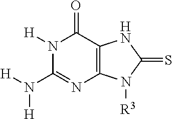 | 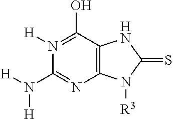 |
| (I-B-2) | |
| 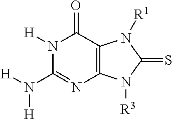 | 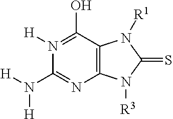 |
| (I-B-3) | |
| 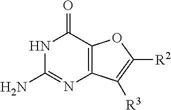 | 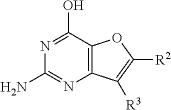 |
| (II-A) | |
| 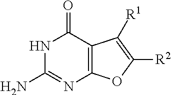 | 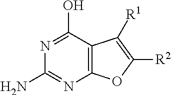 |
| (II-B) | |
| 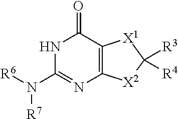 | 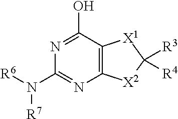 |
| (III) | |
| 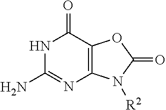 | 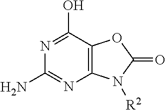 |
| (III-A) | |
| 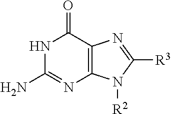 | 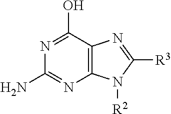 |
| (III-B-1) | |
| 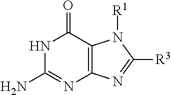 | 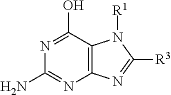 |
| (III-B-2) | |
| 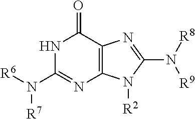 | 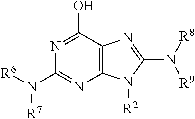 |
| (III-C) | |
| 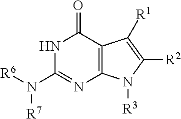 | 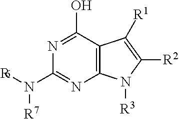 |
| (IV-A) | |
| 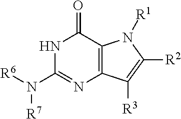 | 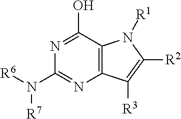 |
| (IV-B) | |
| 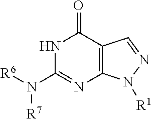 | 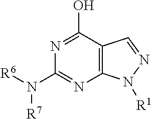 |
| (V-A) | |
| 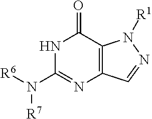 | 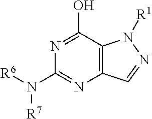 |
| (V-B) | |
| 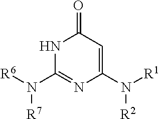 | 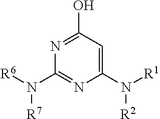 |
| (VI) | |

It is understood that substituents and substitution patterns on the compounds of the invention can be selected by one of ordinary skill in the art to provide compounds that are chemically stable and that can be readily synthesized by techniques known in the art, as well as those methods set forth below, from readily available starting materials. If a substituent is itself substituted with more than one group, it is understood that these multiple groups may be on the same carbon or on different carbons, so long as a stable structure results.

Other features and advantages of the invention will be apparent from the following Detailed Description, the drawings, and the claims.

### BRIEF DESCRIPTION OF THE DRAWINGS

FIG. 1 shows a schematic of BH4 biosynthesis and control. BH4 is synthesized de novo from guanosine triphosphate (GTP) in three steps mediated by GTP cyclohydrolase (GCH-1), 6-pyruvoyltetrahydriobiopterin synthase (PTPS), and sepiapterin reductase (SPR). BH4 is also generated by a separate recycling pathway that converts quinoid BH4 or BH2 to BH4 via enzymatic reduction.

### DETAILED DESCRIPTION

The invention relates to compounds according to Formulas (I)-(VI), or a tautomer, prodrug, pharmaceutically acceptable salt, or pharmaceutical composition thereof, and the use of these compounds and compositions in methods of treatment or to inhibit GTP cyclohydrolase (GCH-1).

Exemplary compounds, or a tautomer, prodrug, or pharmaceutically acceptable salt thereof, include those shown in Table 1.

TABLE 1

| No. | Structure |
|---|---|
| (1) | 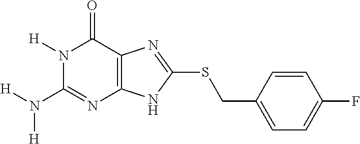 |
| (2) | 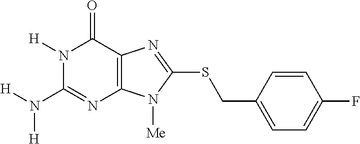 |
| (3) | 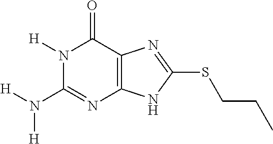 |
| (4) | 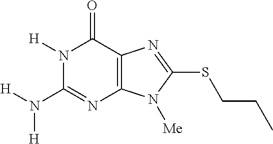 |
| (5) | 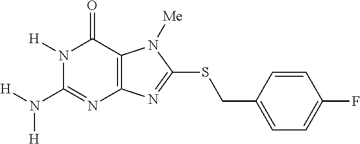 |
| (6) | 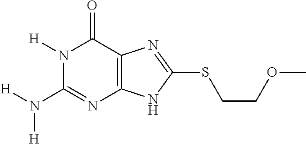 |
| (7) | 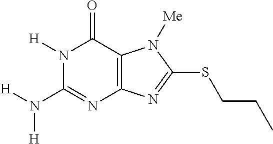 |
| (8) | 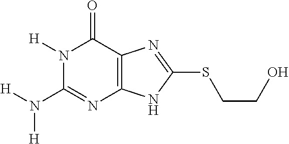 |
| (9) | 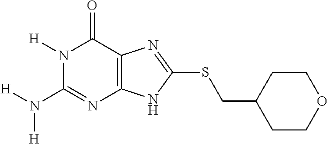 |
| (10) | 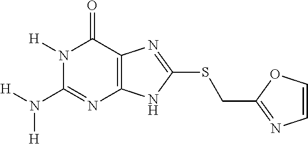 |
| (11) | 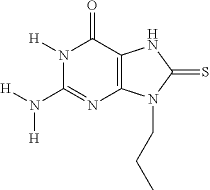 |
| (12) | 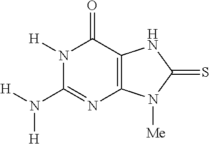 |
| (13) | 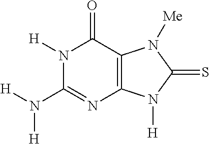 |
| (14) | 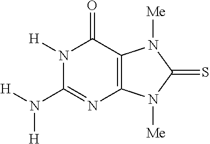 |
| (15) | 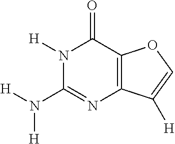 |
| (16) | 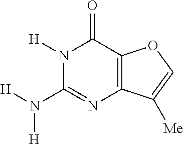 |
| (17) | 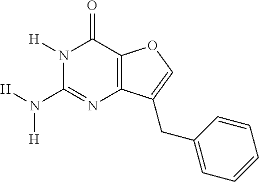 |
| (18) | 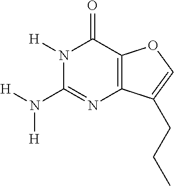 |
| (19) | 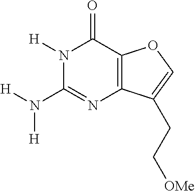 |
| (20) | 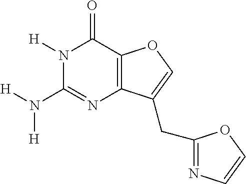 |
| (21) | 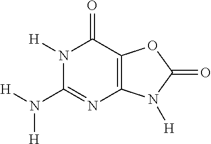 |
| (22) | 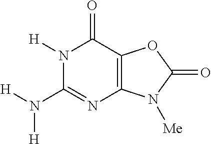 |
| (23) | 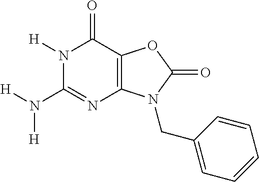 |
| (24) | 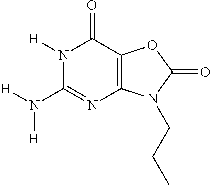 |
| (25) | 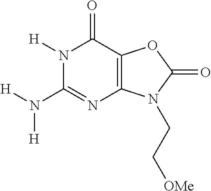 |
| (26) | 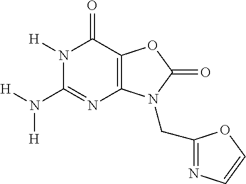 |
| (27) | 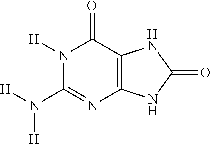 |
| (28) | 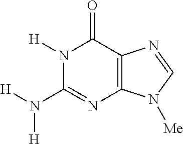 |
| (29) | 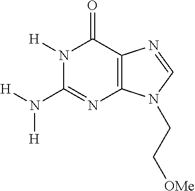 |
| (30) | 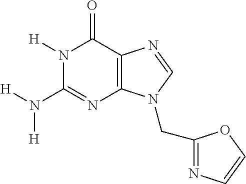 |
| (31) | 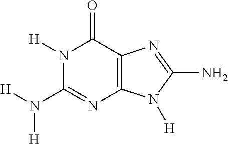 |
| (32) | 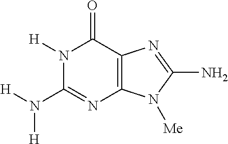 |
| (33) | 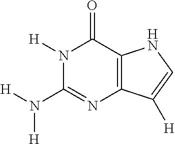 |
| (34) | 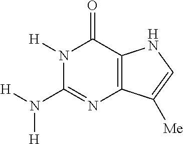 |
| (35) | 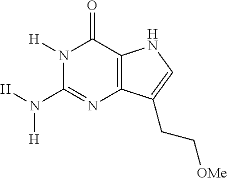 |
| (36) | 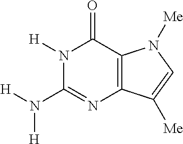 |
| (37) | 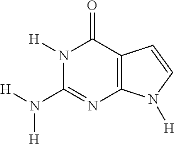 |
| (38) | 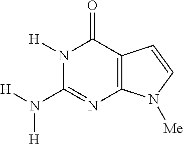 |
| (39) | 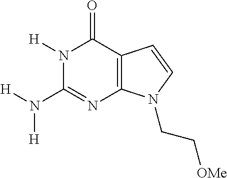 |
| (40) | 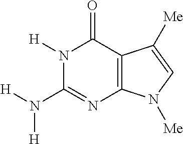 |
| (41) | 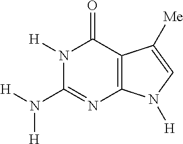 |
| (42) | 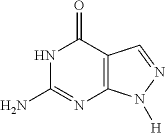 |
| (43) | 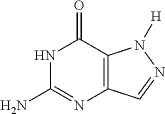 |
| (44) | 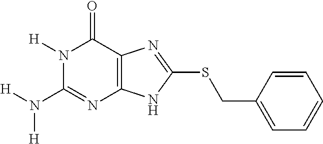 |
| (45) | 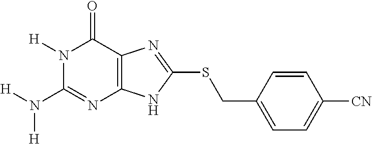 |
| (46) | 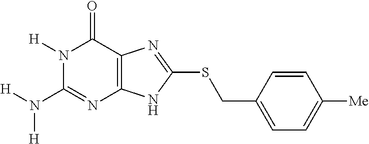 |
| (47) | 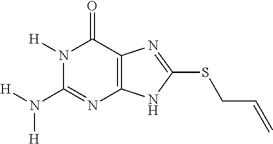 |
| (48) | 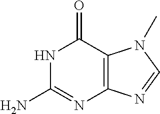 |
| (49) | 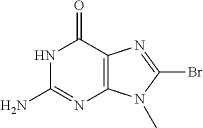 |
| (50) | 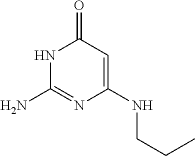 |
| (51) | 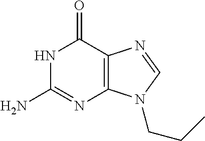 |
| (52) | 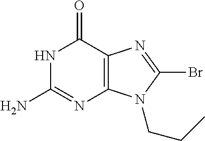 |
| (53) | 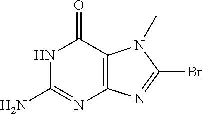 |
| (54) | 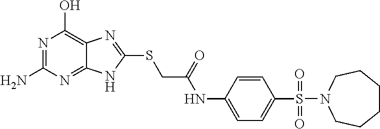 |
| (55) | 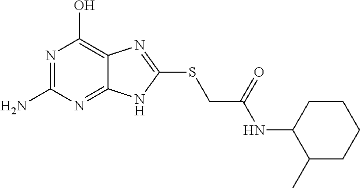 |
| (56) | 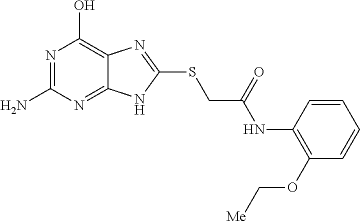 |

### Synthesis

Formula (I) Compounds

Compounds of Formula (I) that include a thioether group can be prepared according to Scheme 1 (see, for example, J. Am. Chem. Soc. 81:1898, 1959), where LG is a suitable leaving group (e.g., a halide such as —Cl, —Br, or —I or a sulfonate such as methylsulfonate (“mesylate” or OMs), trifluoromethylsulfonate (“triflate” or OTf), benzenesulfonate (“besylate” or OBs), p-toluenesulfonate (“tosylate” or OTs), or p- or o-nitrosulfonate (“nosylate” or ONs)) and R is, for example, unsubstituted alkyl or alkyl substituted with a group selected from alkoxy, hydroxy, cycloalkyl, heterocyclyl, aryl, or heteroaryl, each of which may be further substituted:

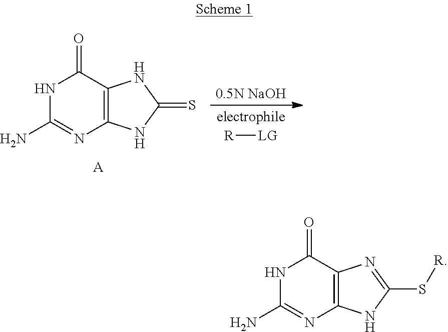

N-substituted thioether compounds of Formula (I) can be prepared according to the method described in Scheme 2 (e.g., J. Am. Chem. Soc. 6:688-692, 1963).

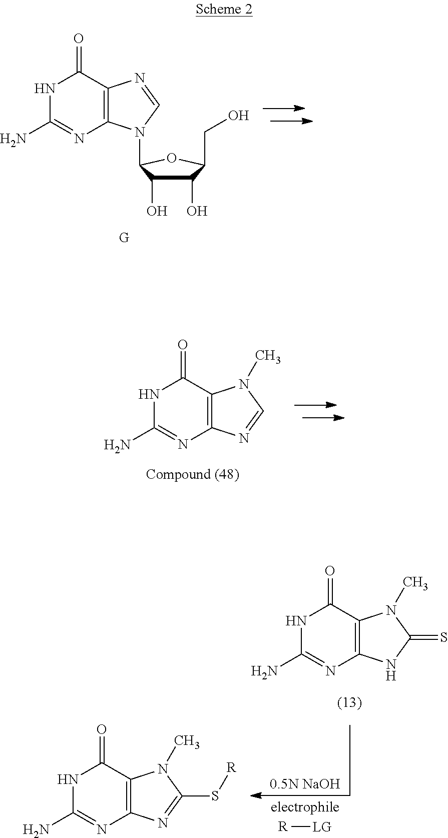

N-substituted thiooxo compounds of Formula (I) can be prepared according to the following procedure of Scheme 3 (e.g., J. Med. Chem. 36:3431, 1993):

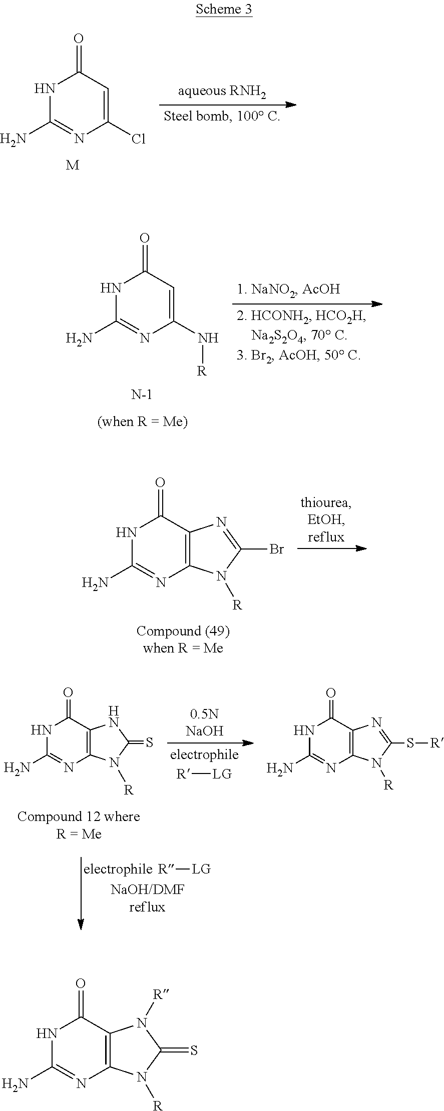

As with the procedure of Scheme 1, different R-LG, R′-LG and R″-LG alkylating groups can be employed in the syntheses of both Schemes 2 and 3 to afford other compounds of Formula (I).

Further compounds of Formula (I), such as Compound (11), can be prepared according to the following Scheme 4.

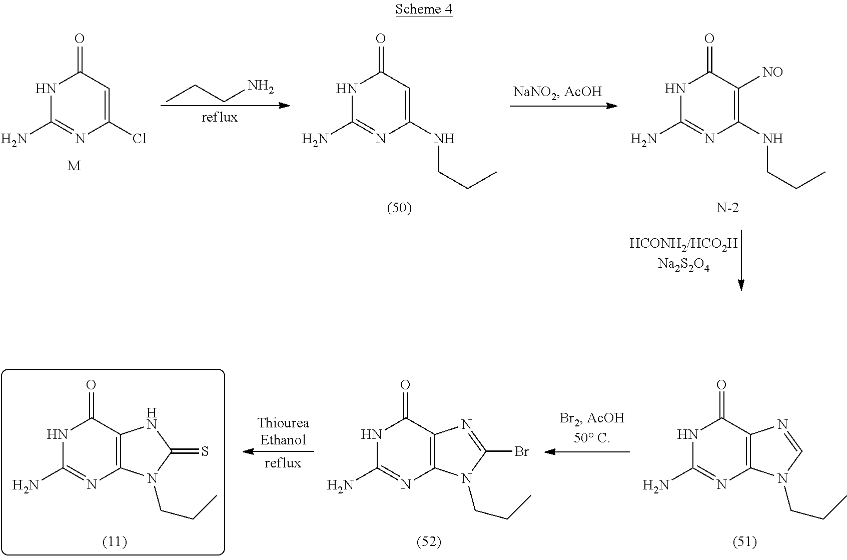

By replacing the n-propylamine used to nucleophilically displace the chloride in the first step with other amine reagents (e.g., other R—NH₂ reagents where R is an unsubstituted alkyl or substituted alkyl such as optionally substituted alkaryl), still other N-alkylated thiooxo compounds of Formula (I) can be obtained.

Formula (II) Compounds

The following procedure (Scheme 5) can be used to prepare compounds according to Formula (II) (e.g., J. Het. Chem. 36:423, 1999) by variation of the initial alkylnitrile starting material RCH₂CN, where R is an unsubstituted alkyl or substituted alkyl such as optionally substituted alkaryl.

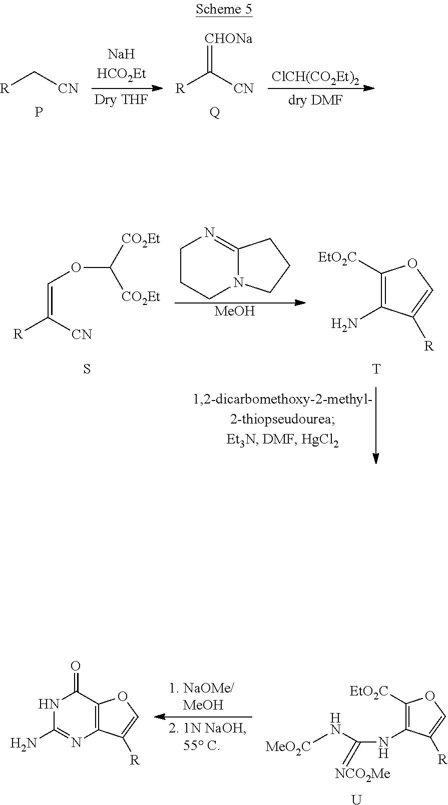

Formula (III) Compounds

Compounds of Formula (III-A) can be prepared according to the following Scheme 6 (e.g., J. Het. Chem. 32:547, 1995).

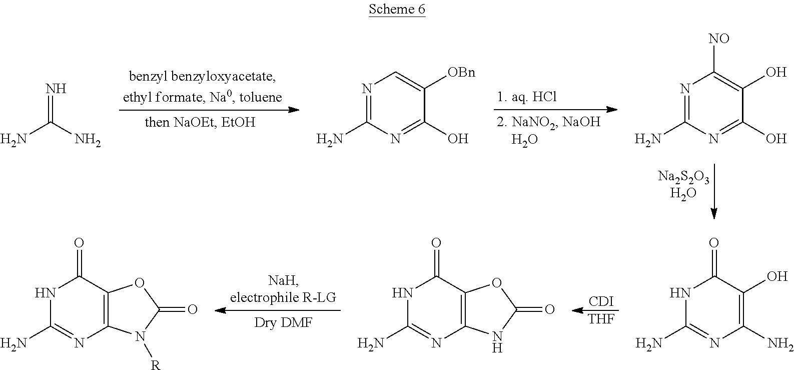

As with the procedure of Scheme 1, different R-LG alkylating groups can be employed in the final N-alkylation to afford various N-substituted oxazolidinone compounds.

Compounds of Formula (III-C) can be prepared according to Scheme 7.

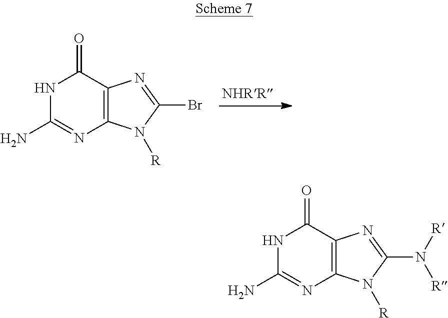

Different compounds of Formula (III-C) can be obtained by variation of the nitrogen source (e.g., amino compounds having the structure NHR′R″, where, for example, each R′ and R″ is, independently, II or optionally substituted alkyl).

Still other compounds of Formula (III-C), such as Compound (32), can be prepared in the manner described in Scheme 8 (e.g., Heterocycles, 22:1978, 1984 and WO 97/12887):

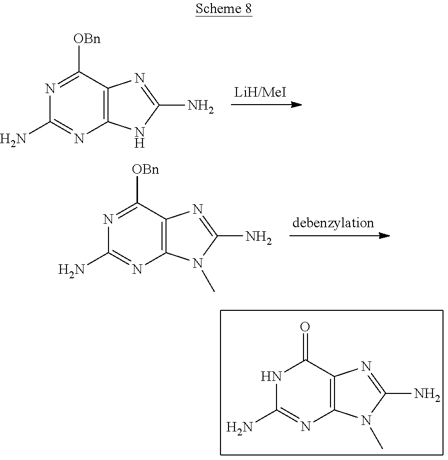

The methyl iodide reagent employed in the first step can be replaced with other electrophilic R-LG reagents as described herein.

Formula (IV) Compounds

The following procedure (Scheme 9) can be used to prepare compounds according to Formula (IV-A) (e.g., any of Compounds (37)-(41); see, for example, J. Med. Chem. 28:1870, 1985 and WO 2005099688).

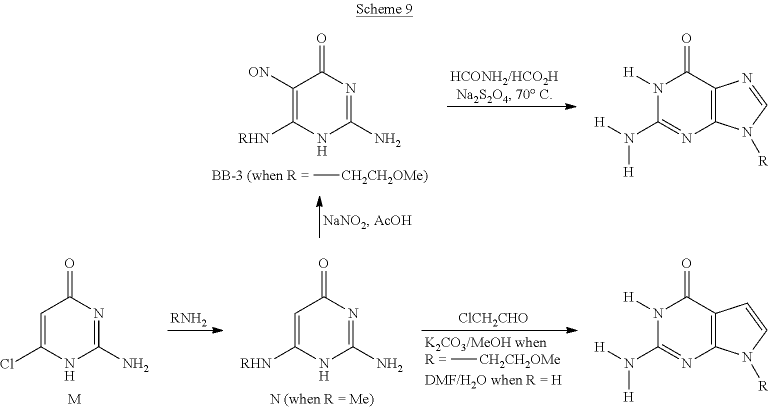

The use of various amine reagents RNH₂, where, for example, R is unsubstituted or substituted alkyl, can afford still other compounds of Formula (IV-A). Intermediate C can be diverted to afford compounds of Formula (III) via nitrosylation and subsequent cyclization. The regioisomer compounds can be prepared by the following Scheme 10, which also employs a nitrosylation/cyclization series of steps:

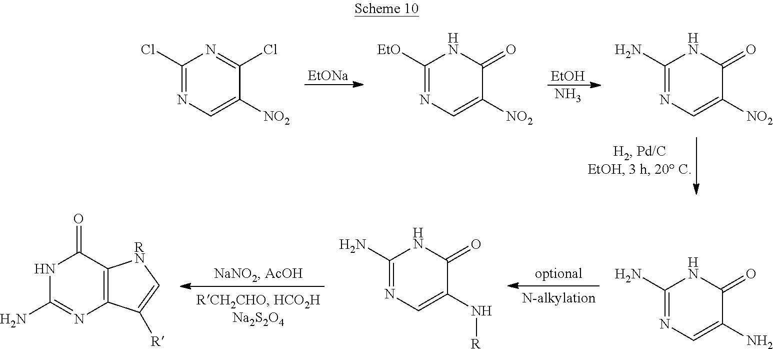

Formula (V) Compounds

Compounds of Formula V can be accessed through multi-step syntheses from monocyclic starting materials (Scheme 11), as shown herein.

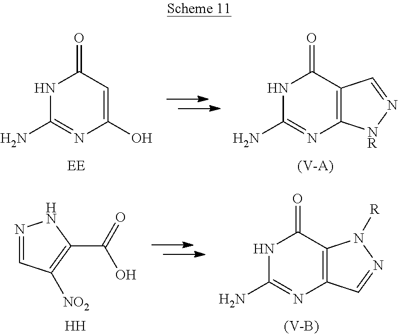

### Pharmaceutical Compositions

The compounds of the invention (e.g., any of Compounds (1)-(56) or a compound according to any of Formulas (I)-(VI)), or tautomers, salts, solvates, or prodrugs thereof, are preferably formulated into pharmaceutical compositions for administration to human subjects in a biologically compatible form suitable for administration in vivo. Accordingly, in another aspect, the present invention provides a pharmaceutical composition comprising a compound of the invention, or a tautomer, salt, solvate, or prodrug thereof, in admixture with a suitable diluent, carrier, or excipient.

The compounds of the invention (e.g., any of Compounds (1)-(56) or a compound according to any of Formulas (I)-(VI)) may be used in the form of the free base, in the form of tautomers, salts, solvates, prodrugs, or pharmaceutical compositions. All forms are within the scope of the invention. In accordance with the methods of the invention, the described compounds, or tautomers, salts, solvates, prodrugs, or pharmaceutical compositions thereof, may be administered to a patient in a variety of forms depending on the selected route of administration, as will be understood by those skilled in the art. The compounds of the invention, or tautomers, salts, solvates, prodrugs, or pharmaceutical compositions thereof, may be administered, for example, by oral, parenteral, buccal, sublingual, nasal, rectal, patch, pump, or transdermal administration and the pharmaceutical compositions formulated accordingly. Parenteral administration includes intravenous, intraperitoneal, subcutaneous, intramuscular, transepithelial, nasal, intrapulmonary, intrathecal, rectal, and topical modes of administration. Parenteral administration may be by continuous infusion over a selected period of time.

A compound of the invention (e.g., any of Compounds (1)-(56) or a compound according to any of Formulas (I)-(VI)), or a tautomer, salt, solvate, or prodrug thereof, may be orally administered, for example, with an inert diluent or with an assimilable edible carrier, or it may be enclosed in hard or soft shell gelatin capsules, or it may be compressed into tablets, or it may be incorporated directly with the food of the diet. For oral therapeutic administration, a compound of the invention, or a tautomer, salt, solvate, or prodrug thereof, may be incorporated with an excipient and used in the form of ingestible tablets, buccal tablets, troches, capsules, elixirs, suspensions, syrups, wafers, and the like.

A compound of the invention (e.g., any of Compounds (1)-(56) or a compound according to any of Formulas (I)-(VI)), or a tautomer, salt, solvate, or prodrug thereof, may also be administered parenterally. Solutions of a compound of the invention can be prepared in water suitably mixed with a surfactant, such as hydroxypropylcellulose. Dispersions can also be prepared in glycerol, liquid polyethylene glycols, DMSO and mixtures thereof with or without alcohol, and in oils. Under ordinary conditions of storage and use, these preparations may contain a preservative to prevent the growth of microorganisms. Conventional procedures and ingredients for the selection and preparation of suitable formulations are described, for example, in Remington's Pharmaceutical Sciences (2003-20th edition) and in The United States Pharmacopeia: The National Formulary (USP 32-NF 27), published in 2008.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that may be easily administered via syringe.

Pharmaceutical compositions for nasal administration may conveniently be formulated as aerosols, drops, gels, and powders. Aerosol formulations typically include a solution or fine suspension of the active substance in a physiologically acceptable aqueous or non-aqueous solvent and are usually presented in single or multidose quantities in sterile form in a sealed container, which can take the form of a cartridge or refill for use with an atomizing device. Alternatively, the sealed container may be a unitary dispensing device, such as a single dose nasal inhaler or an aerosol dispenser fitted with a metering valve which is intended for disposal after use. Where the dosage form comprises an aerosol dispenser, it will contain a propellant, which can be a compressed gas, such as compressed air or an organic propellant, such as fluorochlorohydrocarbon. The aerosol dosage forms can also take the form of a pump-atomizer.

Compositions suitable for buccal or sublingual administration include tablets, lozenges, and pastilles, where the active ingredient is formulated with a carrier, such as sugar, acacia, tragacanth, or gelatin and glycerine. Compositions for rectal administration are conveniently in the form of suppositories containing a conventional suppository base, such as cocoa butter.

The compounds of the invention may be administered to an animal, e.g., a human, alone or in combination with pharmaceutically acceptable carriers, as noted above, the proportion of which is determined by the solubility and chemical nature of the compound, chosen route of administration, and standard pharmaceutical practice.

The dosage of the compounds of the invention, and/or compositions comprising a compound of the invention, can vary depending on many factors, such as the pharmacodynamic properties of the compound; the mode of administration; the age, health, and weight of the recipient; the nature and extent of the symptoms; the frequency of the treatment, and the type of concurrent treatment, if any; and the clearance rate of the compound in the animal to be treated.

One of skill in the art can determine the appropriate dosage based on the above factors. The compounds of the invention may be administered initially in a suitable dosage that may be adjusted as required, depending on the clinical response. Generally, dosage levels of between 0.1 μg/kg to 100 mg/kg of body weight are administered daily as a single dose or divided into multiple doses. Desirably, the general dosage range is between 250 μg/kg to 5.0 mg/kg of body weight per day. Wide variations in the needed dosage are to be expected in view of the differing efficiencies of the various routes of administration. For instance, oral administration generally would be expected to require higher dosage levels than administration by intravenous injection. Variations in these dosage levels can be adjusted using standard empirical routines for optimization, which are well known in the art. In general, the precise therapeutically effective dosage will be determined by the attending physician in consideration of the above identified factors.

### Kits

Any of the compounds or pharmaceutical compositions of the invention (e.g., any of Compounds (1)-(56) or a compound according to any of Formulas (I)-(VI)) can be used together with a set of instructions, i.e., to form a kit. The kit may include instructions for use of the compounds of the invention in a screening method or as a therapy as described herein. For example, the instructions may provide dosing and therapeutic regimes for use of the compounds of the invention to reduce pain, including any type of pain described herein.

### Inhibitors of GCH-1

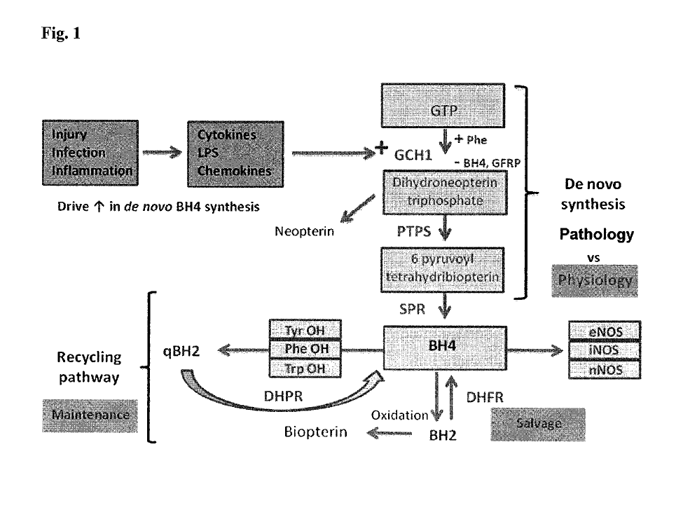

The compounds and compositions described herein can be used to inhibit GCH-1, which is the rate limiting enzyme in the transformation of GTP to BH4. BH4 is an essential co-factor required for normal function of several enzyme and neurotransmitter systems: phenylalanine hydroxylase, tyrosine hydroxylase, tryptophan hydroxylase, and the 3 nitric oxide synthases (NOS) subtypes all rely on BH4 allosteric regulation (Thony et al., Biochem. J. 347:1-16, 2000). BH4 is synthesized from guanosine triphosphate (GTP) in three tightly regulated steps by GCH-1,6-pyruvoyltetrahydriobiopterin synthase (PTPS), and sepiapterin reductase (SPR) (FIG. 1).

Two of the enzymes involved in de novo BH4 synthesis, GCH-1 and SPR, are up-regulated in preclinical pain models, and reducing the activity of these enzymes leads to preclinical pain relief (Tegeder et al., Nature Medicine 12:1269-1277, 2006). Subsequent human genetic studies examining the correlation of altered pain responses with a specific GCH-1 haplotype translate the preclinical observations between the GCH-1 pathway in animals and pain responses in humans (Max et al., Nat. Rev. Drug. Disc. 7:647-58, 2008). Accordingly, agents that reduce de novo BH4 synthesis (e.g., via direct active site inhibition of GCH-1) can be used in the prevention or treatment of pain.

Compounds that inhibit GCH-1 can be identified using the methods described herein and those known in the art (e.g., those described in U.S. Ser. No. 10/987,289 or Kolinsky et al., J. Biol. Chem. 279:40677-40682, 2004, each of which is hereby incorporated by reference). For example, GCH-1 activity may be assessed by measuring the release of labeled formic acid originating from a labeled hydrogen atom of GTP and separation of formic acid from GTP by charcoal (Viveros et al., Science 213:349, 1981). HPLC-based methods, however, are superior to the radioactive method in that HPLC allows determination of the product. For measuring GCH-1 activity, the tissue or cell homogenate containing GCH-1 is incubated with excess GTP (substrate) in the presence of EDTA to ensure that the product 7,8 dihydropterin triphosphate is not further metabolized by the downstream PTPS which requires Mg²⁺ to operate. The reaction is stopped by the addition of HCl and iodine. This also results in oxidation of the labile 7,8-dihydroneopterin triphosphate to the more stable neopterin triphosphate. Neopterin triphosphate may be analyzed directly by ion-pair HPLC and fluorescence detection. Alternatively, the mixture is treated with NaOH and alkaline phosphatase to yield neopterin which can be analyzed using reversed-phase HPLC with fluorescence detection, immunoassay, or direct fluorescence in the case of “pure” samples (such as in vitro kinase assay or CSF).

Screening of multiple compounds can be assessed, for example, by measuring GCH-1 activity as described herein using a 96 well-based enzyme assay where purified recombinant GCH-1 is incubated together with substrate and the potential inhibitor followed by oxidation and measurement of neopterin with a fluorescence ELISA reader. Neopterin shows intense fluorescence and can be directly measured.

Assays may also be based on BH4 measurement. BH4 shows no intense fluorescence because the rings of the molecule are not in the fully oxidized, aromatic state. To circumvent this, a differential oxidization method in which dihydrobiopterin and BH4 are measured following their oxidation to biopterin may be used, with a limit of detection of 0.3 pmol for biopterin with fluorescence (Fukushima and Nixon, Anal. Biochem. (1980) 102: 176-188). Assays for measuring the activity of GCH-1 or levels of biopterin are described, for example, by Kaneko et al., Brain Res. Brain Res. Protoc. 8:25-31, 2001; Ota et al., J. Neurochem. 67:2540-2548, 1996; Bräutigam et al., Physiol. Chem. 363:341-343, 1982; Curtius et al., Eur. J. Biochem. 148:413-419, 1985; Stea et al., J. Chromatogr. 168:385-393, 1979; Werner et al., J. Chromatogr. 684:51-58, 1996; Werner et al., Methods Enzymol. 281:53-61, 1997; Nagatsu et al., Anal. Biochem. 110:182-189, 1981; and Geller et al., Biochem. Biophys. Res. Commun. 276:633-41, 2000, each of which is hereby incorporated by reference.

Although not necessary, if desired, candidate GCH-1 inhibitors can be tested for efficacy in any standard animal model of pain. Various models test the sensitivity of normal animals to intense or noxious stimuli (physiological or nociceptive pain). These tests include responses to thermal, mechanical, or chemical stimuli. Thermal stimuli usually involve the application of hot stimuli (typically varying between 42-55° C.) including, for example: radiant heat to the tail (the tail flick test), radiant heat to the plantar surface of the hindpaw (the Hargreaves test), the hotplate test, and immersion of the hindpaw or tail into hot water. Immersion in cold water, acetone evaporation, or cold plate tests may also be used to test cold pain responsiveness. Tests involving mechanical stimuli typically measure the threshold for eliciting a withdrawal reflex of the hindpaw to graded strength monofilament von Frey hairs or to a sustained pressure stimulus to a paw (e.g., the Ugo Basile analgesiometer). The duration of a response to a standard pinprick may also be measured. When using a chemical stimulus, the response to the application or injection of a chemical irritant (e.g., capsaicin, mustard oil, bradykinin, ATP, formalin, acetic acid) to the skin, muscle joints or internal organs (e.g., bladder or peritoneum) is measured.

In addition, various tests assess pain sensitization by measuring changes in the excitability of the peripheral or central components of the pain neural pathway. In this regard, peripheral sensitization (i.e., changes in the threshold and responsiveness of high threshold nociceptors) can be induced by repeated heat stimuli as well as the application or injection of sensitizing chemicals (e.g., prostaglandins, bradykinin, histamine, serotonin, capsaicin, or mustard oil). Central sensitization (i.e., changes in the excitability of neurons in the central nervous system induced by activity in peripheral pain fibers) can be induced by noxious stimuli (e.g., heat), chemical stimuli (e.g., injection or application of chemical irritants), or electrical activation of sensory fibers.

Various pain tests developed to measure the effect of peripheral inflammation on pain sensitivity can also be used, if desired, to confirm the efficacy of GCH-1 inhibitors (Stein et al., Pharmacol. Biochem. Behav. (1988) 31: 445-451; Woolf et al., Neurosci. (1994) 62: 327-331). Additionally, various tests assess peripheral neuropathic pain using lesions of the peripheral nervous system. One such example is the “axotomy pain model” (Watson, J. Physiol. (1973) 231:41). Other similar tests include the SNL test which involves the ligation of a spinal segmental nerve (Kim and Chung Pain (1992) 50: 355), the Seltzer model involving partial nerve injury (Seltzer, Pain (1990) 43: 205-18), the spared nerve injury (SNI) model (Decosterd and Woolf, Pain (2000) 87:149), chronic constriction injury (CCl) model (Bennett (1993) Muscle Nerve 16: 1040), tests involving toxic neuropathies such as diabetes (streptozocin model), pyridoxine neuropathy, taxol, vincristine, and other antineoplastic agent-induced neuropathies, tests involving ischaemia to a nerve, peripheral neuritis models (e.g., CFA applied peri-neurally), models of post-herpetic neuralgia using HSV infection, and compression models.

In all of the above tests, outcome measures may be assessed, for example, according to behavior, electrophysiology, neurochemistry, or imaging techniques to detect changes in neural activity. Furthermore, several pain tests that mimic central neuropathic pain involve lesions of the central nervous system including, for example, spinal cord injury (e.g., mechanical, compressive, ischemic, infective, or chemical). In these particular tests, outcome measures are the same as those used for peripheral neuropathic pain.

### Therapy

The methods of this invention are useful for the diagnosis, treatment, reduction, or prevention of various forms of pain.

Pain can take a variety of forms depending on its origin. Pain may be described as being peripheral neuropathic if the initiating injury occurs as a result of a complete or partial transection of a nerve or trauma to a nerve plexus. Alternatively, pain is described as being central neuropathic following a lesion to the central nervous system, such as a spinal cord injury or a cerebrovascular accident. Inflammatory pain is a form of pain that is caused by tissue injury or inflammation (e.g., in postoperative pain or rheumatoid arthritis). Following a peripheral nerve injury, symptoms arc typically experienced in a chronic fashion, distal to the site of injury and are characterized by hyperesthesia (enhanced sensitivity to a natural stimulus), hyperalgesia (abnormal sensitivity to a noxious stimulus), allodynia (widespread tenderness associated with hypersensitivity to normally innocuous tactile stimuli), and/or spontaneous burning or shooting lancinating pain. In inflammatory pain, symptoms are apparent, at least initially, at the site of injury or inflamed tissues and typically accompany arthritis-associated pain, musculo-skeletal pain, and postoperative pain. Nociceptive pain is the pain experienced in response to a noxious stimulus, such as a needle prick or during trauma or surgery. Functional pain refers to conditions in which there is no obvious peripheral pathology or lesion to the nervous system. This particular form of pain is generated by abnormal function of the nervous system and conditions characterized by such pain include fibromyalgia, tension-type headache, and irritable bowel syndrome. The different types of pain may coexist or pain may be transformed from inflammatory to neuropathic during the natural course of the disease, as in post-herpetic neuralgia.

The methods of this invention are useful for the diagnosis, treatment, reduction, or prevention of various forms of pain, namely inflammatory pain, nociceptive pain, functional pain, and neuropathic pain, whether acute or chronic. Exemplary conditions that may be associated with pain include, for example, soft tissue, joint, bone inflammation and/or damage (e.g., acute trauma, osteoarthritis, or rheumatoid arthritis), myofascial pain syndromes (fibromylagia), headaches (including cluster headache, migraine, and tension type headache), myocardial infarction, angina, ischemic cardiovascular disease, post-stroke pain, sickle cell anemia, peripheral vascular occlusive disease, cancer, inflammatory conditions of the skin or joints, diabetic neuropathy, and acute tissue damage from surgery or traumatic injury (e.g., burns, lacerations, or fractures). The present invention is also useful for the treatment, reduction, or prevention of musculo-skeletal pain (after trauma, infections, and exercise), neuropathic pain caused by spinal cord injury, tumors, compression, inflammation, dental pain, episiotomy pain, deep and visceral pain (e.g., heart pain, bladder pain, or pelvic organ pain), muscle pain, eye pain, orofacial pain (e.g., odontalgia, trigeminal neuralgia, glossopharyngeal neuralgia), abdominal pain, gynecological pain (e.g., dysmenorrhea and labor pain), pain associated with nerve and root damage due to trauma, compression, inflammation, toxic chemicals, metabolic disorders, hereditary conditions, infections, vasculitis and autoimmune diseases, central nervous system pain, such as pain due to spinal cord or brain stem damage, cerebrovascular accidents, tumors, infections, demyelinating diseases including multiple sclerosis, low back pain, sciatica, and post-operative pain. Conditions that are amenable to treatment according to the present invention are described in detail, for example, in U.S. Ser. No. 10/987,289 and 11/584,449, as well as U.S. Pat. No. 6,593,331, each of which are hereby incorporated by reference.

### Combination Therapy

The compounds of the present invention (e.g., any of Compounds (1)-(56) or a compound according to any of Formulas (I)-(VI)), or a tautomers, salt, solvate, prodrug, or pharmaceutical composition thereof, may be administered either alone or in combination with a second therapeutic agent, such as an analgesic agent used in the treatment of nociception, inflammatory, functional, or neuropathic pain. According to this invention, the second therapeutic agent may or may not produce a therapeutic effect when administered on its own, but results in such an effect (e.g., pain reduction) when administered with the composition of the invention.

Exemplary analgesic agents include, without limitation, nonsteroidal anti-inflammatory agents (NSAIDs) (e.g. rofexocib, celecoxib, valdecoxib, paracoxib, salicylic acid, acetominophen, diclofenac, piroxican indomethacin, ibuprofen, and naproxen), opioid analgesics (e.g., propoxyphene, meperidine, hydromorphone, hydrocodone, oxycodone, morphine, codeine, and tramodol), NMDA antagonist analgesics (e.g., 2-piperidino-1 alkanol derivatives, ketamine, dextormethorphan, eliprodil, or ifenprodil), anesthetic agents (e.g., nitrous oxide, halothane, fluothane), local anesthetics (lidocaine, etidocaine, ropivacaine, chloroprocaine, sarapin, and bupivacaine), benzodiazepines (diazepam, chlordiazepoxide, alprazolam, and lorazepam), capsaicin, tricyclic antidepressants (e.g., amitriptyline, perphanazine, protriptyline, tranylcypromine, imipramine, desimipramine, and clomipramine), skeletal muscle relaxant analgesics (flexeril, carisoprodol, robaxisal, norgesic, and dantrium), migraine therapeutic agents (e.g., elitriptan, sumatriptan, rizatriptan, zolmitriptan, and naratriptan), anticonvulsants (e.g., phenyloin, lamotrigine, pregabalin, carbamazepine, oxcarbazepine, topiramate, valproic acid, and gabapentin), baclofen, clonidine, mexilitene, diphenyl-hydramine, hydroxysine, caffeine, prednisone, methylprednisone, decadron, paroxetine, sertraline, fluoxetine, tramodol, ziconotide, and levodopa.

If desired, the mammal being treated may be administered with more than one agent that inhibits the production of BH4 (e.g., those described in U.S. Ser. No. 10/987,289, hereby incorporated by reference). Optionally, the composition of the invention may contain more than one such inhibitor. Alternatively, the mammal may further be administered with specific inhibitors of enzymes that function downstream of BH4, in addition to the composition of the invention.

The following non-limiting examples are illustrative of the present invention.

### EXAMPLES

### Synthesis of Formula (I) Compounds

### Synthesis of Compound (1)

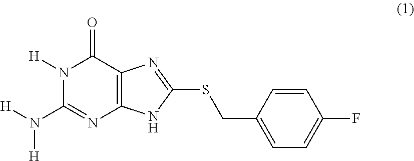

Compound (1) was synthesized according to Scheme 1 in the following manner (see also J. Am. Chem. Soc. 81:1898, 1959).

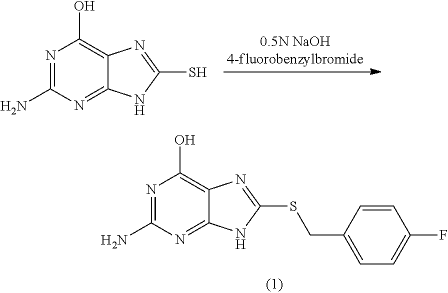

To a solution of 2-amino-6-hydroxy-8-mercaptopurine (1 g, 5.46 mmol) in 0.5 N NaOH (23.4 mL) was added slowly 4-fluorobenzylbromide (1.13 g, 5.97 mmol), and the reaction stirred for 1 hour. At this time, the precipitated solid was filtered. The filtrate was cooled to 0° C., acidified with acetic acid (pH ˜5.0), and the precipitated solid was collected by filtration. The precipitate was again dissolved in 0.5 aqueous NaOH solution (6 mL), washed with EtOAc (2×10 mL), and acidified with acetic acid (pH ˜5.0). The precipitate was collected by filtration, washed with water (20 mL) and acetone (20 mL), and then dried to give Compound (1) as a pale yellow solid. ¹H NMR (300 MHz, CDCl₃): δ 12.5 (bs, 1H, D₂O exchangeable), 10.5 (bs, 1H, D₂O exchangeable), 7.41-7.38 (m, 2H), 7.14-7.10 (m, 2H), 6.29 (bs, 2H, D₂O exchangeable), 4.37 (s, 2H), Mass (M+H)⁺=292 (100), IR (KBr) 3334, 1670, 1344 cm⁻¹.

### Synthesis of Compounds (3), (6), (8), (9), and (10)

The procedure described in Scheme 1 has also been used to prepare the following compounds according to the following general procedure (see also Scheme 12).

General Procedure A

To a solution of Intermediate A (1 mmol) in a 2:3 mixture of 0.5 aqueous NaOH(2 mL)/H₂O(3 ml) was added the corresponding alkyl halides (e.g., any of Intermediates B, C, D, E, F, or G; 2 mmol) respectively at room temperature (26° C.) and stirred for 2 hours. The reaction mixture was then treated with AcOH (5 ml), stirred for ˜15-20 minutes. The precipitated solid was collected by filtration, washed thoroughly with water (15-20 mL), and dried to obtain the corresponding crude S-alkylated product, which was further purified by preparative HPLC (Column: Zodiac sil 120-5-C-18, 50×20 mm, 10μ, Mobile phase, A; 0.01M NH₄OAc, B: MeOH; (T/% B): 0/5, 10/90, 20/90, 20.1/0.5, Flow rate: 20 mL/minute, Diluents: DMSO and MeOH).

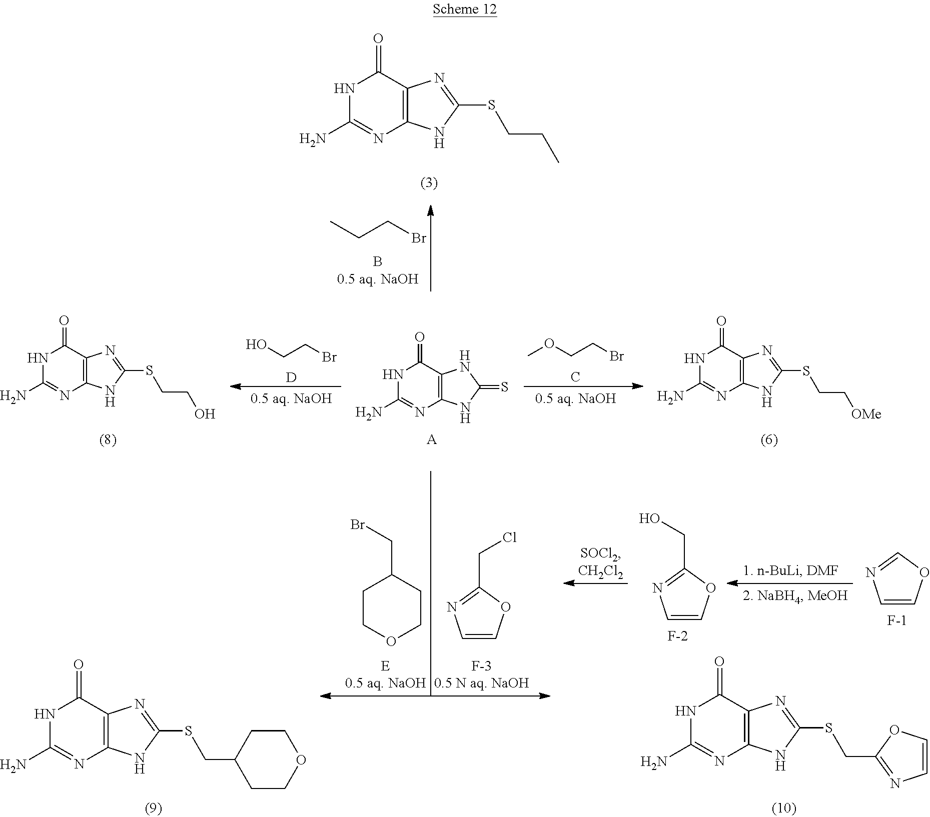

Synthesis of Compound (3)

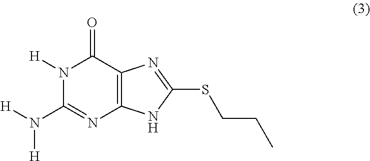

Compound (3) was prepared according to the General Procedure A, using Intermediate B as the alkylating agent. ¹H NMR (400 MHz, DMSO-d₆):12.50 (br. s, exchanged with D₂O, 1H), 10.50 (br. s, exchanged with D₂O, 1H), 6.30 (br. s, exchanged with D₂O, 2H), 3.08 (br. s, 2H), 1.65 (quintet, J=, 6.8 Hz, 2H), 0.96 (t, J=7.2 Hz, 3H). LCMS (Column: Zodiacsil 120-5-C-18, (4.6×50 mm), Mobile phase, (A: 0.01M HCOONH₄, B: MeOH, T/% B: 0/5, 10/90, 10.1/5), Flow rate: 1.0 ml/min, Diluents: H₂O+MeOH)): 99.04% at 214 nm, 98.2 at 254 nm; Rₜ=5.82; m/z=225.8.

Synthesis of Compounds (6), (8), and (9)

Compounds (6)-(8) were prepared according to General Procedure A and are shown in Table 2.

TABLE 2

| Compound | Alkylating Agent |
|---|---|
| 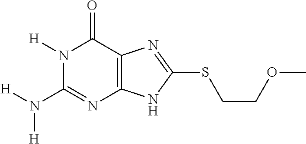 | Intermediate C |
| (6) | |
| 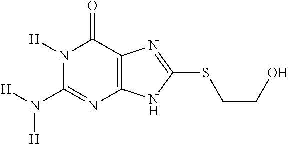 | Intermediate D |
| (8) | |
| 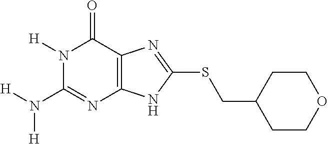 | Intermediate E |
| (9) | |

Synthesis of Compound (10)

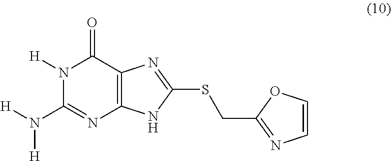

The following procedure is described in Scheme 12 and is used to prepare the alkylating agent F-3.

At −78° C., a solution of F-1 (800 mg, 11.58 mmol) in THF was treated with 1.6 M solution of n-BuLi in hexanes (7.2 mL, 11.58 mmol). The reaction was stirred for 1 hour, and then a solution of DMF (0.89 mmol, 11.58 mmol) in THF (8 mL) was added. The reaction was warmed to room temperature and stirred overnight. The reaction mixture was treated with MeOH, filtered through Dowex 50W×8-200 resin (H⁺ form, 1:1 ratio to oxazole F-1), and washed with MeOH. The combined filtrate was concentrated to obtain the crude aldehyde F-2a,

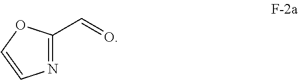

This material was then purified by column chromatography (100-200 mesh silica gel, 2% Et₂O/CH₂Cl₂) to obtain the purified aldehyde (900 mg, 80%) as a light brown liquid (R_{f}=0.7(2% Et₂O/CH₂Cl₂)). This product was used in the next step without any characterization.

To a solution of F-2a (90 mg, 9.28 mmol) in MeOH (25 mL) was added NaBH₄ (420 mg, 11.14 mmol) at −0° C. The mixture was warmed to room temperature and stirred for 1 hour. The reaction mixture was treated with water (25 mL), extracted with EtOAc, washed with brine solution (50 mL), dried (Na₂SO₄), filtered, and evaporated to obtain crude compound F-2. This material was subjected to column chromatography (100-200 mesh size silica gel, 50% EtOAc/petroleum ether) to obtain the purified product F-2 (160 mg, 14%) as colorless liquid. R_{f}=0.4 (50% EtOAc/petroleum ether). ¹H-NMR (400 MHz, CDCl₃): 7.64 (s, 1H), 7.09 (s, 1H), 4.75 (s, 2H). Mass (m/z, APCI positive mode): 100.3 (M⁺+1).

To a solution of compound F-2 (100 mg, 1.0 mmol) in CH₂Cl₂ (5 mL) was added SOCl₂ (420 mg, 11.14 mmol) at −0° C. The mixture was warmed to room temperature and stirred for 1 hour. The reaction mixture was treated with water (25 mL), extracted with EtOAc (2×30 mL), washed with brine solution (60 mL), dried (Na₂SO₄), filtered, and evaporated to obtain crude compound F-3 (125 mg, 14%) as colorless liquid. R_{f}=0.6 (5% EtOAc/petroleum ether). ¹H-NMR (400 MHz, DMSO-d₆): 8.19 (s, 1H), 7.27 (s, 1H), 4.89 (s, 2H). Mass (m/z, APCI positive mode): 118 (M⁺+1). The reagent was used without further purification.

Compound (10) was prepared according to the General Procedure A, using Intermediate F-3 as the alkylating agent. ¹H-NMR (400 MHz, DMSO-d₆): 12.62 (br. s, exchanged with D₂O, 1H), 10.52 (br. s, exchanged with D₂O, 1H), 8.04 (s, 1H), 7.13 (s, 1H), 6.34 (br. s, exchanged with D₂O, 2H), 4.51 (br. s, 2H). LCMS (Column: Zodiacsil 120-5-C-18, (4.6×50) mm; Mobile phase: A: 0.01M HCOONH₄, B: MeOH, T/% B: 0/5, 10/90, 10.1/5, Flow rate: 1.0 mL/min, diluents: mobile phase+MeOH): 97.42% at 214 nm, 96.05% at 254 nm; m/z=241.8, Rₜ=2.54 min.

Synthesis of Compounds (54)-(56)

Compounds (54)-(56) can be prepared according to the procedure described in Scheme 1 by using analogous syntheses to those described for the preparation of Compounds (3), (6), (8), (9), and (10) described herein.

Synthesis of Compounds (5), (7), (13), and (48)

The procedure described in Scheme 2 was used to prepare Compounds (5), (7), (13), and (48) (see also Scheme 13).

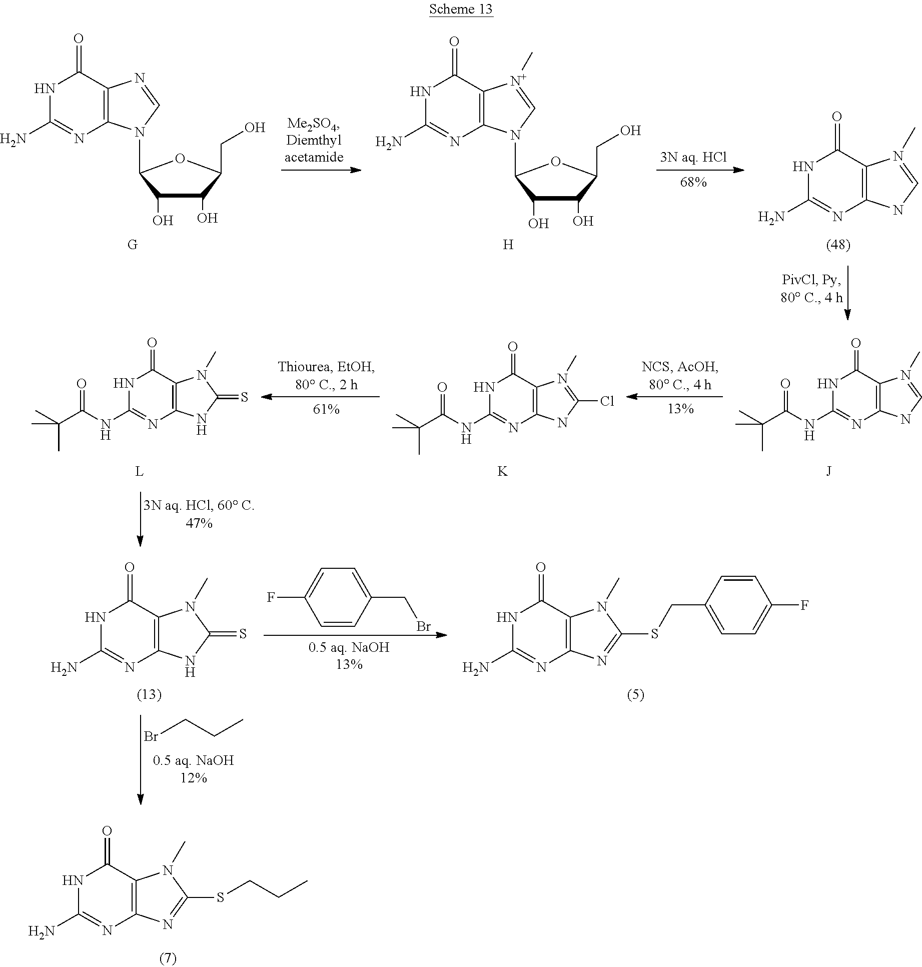

Synthesis of Compounds (48) and (13)

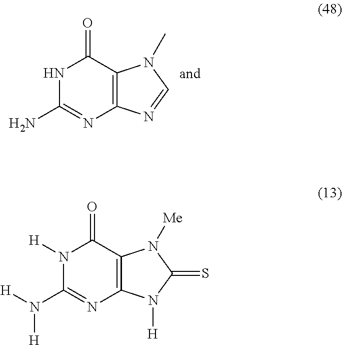

Preparation of Intermediate H

To a solution of intermediate G (10 g, 35.3 mmol) in dimethylacetamide (50 mL) was added Me₂SO₄ (10 mL, 106 mmol, 3 equivalents) at room temperature. The reaction was stirred for 6 hours; during this time, the formation of a clear solution was observed. The product (Intermediate J) was used as such for the next step without any further characterization and purification.

Preparation of Compound (48)

A solution of Intermediate H (10 g, 35.3 mmol) in 3N aq HCl (100 mL) was stirred at 100° C. for 1 hour. The reaction mixture was cooled to 0° C., treated with aqueous NH₃, and adjusted to pH=8. The precipitated solid was collected by filtration, washed with water (300 mL) and EtOH (300 mL), and dried in vacuo to obtain Compound (48) (4 g, 68%) as an off white solid liquid. R_{f}=0.5 (20% MeOH/CHCl₃/aq. NH₃). ¹H-NMR (400 MHz, DMSO-d₆) δ 8.60 (br. hump, exchanged with D₂O, 1H), 6.09 (s, exchanged with D₂O, 2H), (s, 1H), 3.82 (s, 1H). Mass (m/z, APCI, positive mode): 166.6 (M⁺+1). HPLC: (Column: Acquity; HPLC BEH; C-18 (100×2.1 mm) 1.7μ, A: 0.025% TFA (Aqueous), B: 0.025% TFA (Acetonitrile), T/% B: 0/10, 4/80, 6/80, 6.1/10, Flow rate: 0.3 mL/min, Diluent: A:B (9:1)), Rₜ=0.927 min, 98.35 (215 nm), 98.56 (254 nm), 98.61 (Max plot).

Preparation of Intermediate J

To a solution of Compound (48) (1 g, 6.06 mmol) in pyridine (10 mL) was added pivaloyl chloride (1.1 mL, 9.1 mmol), and the reaction was stirred at 80° C. for 3 hours. The reaction mixture was cooled to room temperature (0° C.), treated with cold water, extracted with EtOAc (2×50 mL), washed with brine solution (30 mL), dried (Na₂SO₄), and concentrated to obtain crude Intermediate J, which was subjected to column chromatography (100-200 mesh silica gel, 30% EtOAc/petroleum ether) to obtain the purified product (300 mg, 20%) as anoff white solid. R_{f}=0.6 (50% EtOAc/petroleum ether). Mass (m/z, APCI, positive mode): 250.1 (M⁺+1).

Preparation of Intermediate K

To a solution of Intermediate J (1 g, 4.01 mmol) in AcOH (5 mL) was added N-chlorosuccinimide (NCS; 800 mg, 6 mmol), and the resulting mixture was stirred at 80° C. for 6 hours. The reaction mixture was cooled to room temperature (0° C.), H₂O was added (100 mL), and the mixture was extracted with EtOAc (2×100 mL), washed with brine solution (20 mL), dried (Na₂SO₄), filtered, and concentrated to obtain crude Intermediate K, which was subjected to column chromatography (100-200 mesh silica gel, 10% EtOAc/petroleum ether) to afford the product (150 mg, 13%) as off white powder. R_{f}=0.6 (30% EtOAc/petroleum ether). ¹H-NMR (400 MHz, DMSO-d₆) δ 12.38 (br. s, exchanged with D₂O, 1H), 11.17 (br. hump, exchanged with D₂O, 1H), 3.89 (s, 1H), 1.16 (s, 9H). Mass (m/z, APCI, positive mode and negative mode): 284.1 (M⁺+1) & 282.0 (M⁺−1).

Preparation of Intermediate L

To a solution of Intermediate K (500 mg, 3.53 mmol) in EtOH (5 mL) was added thiourea (537 mg, 7.06 mmol), and the resulting mixture was stirred at 80° C. for 4 hours. The reaction mixture was cooled to 0° C. and concentrated to dryness. H₂O was added (50 mL), and the mixture was extracted with EtOAc (2×50 mL), washed with brine solution (20 mL), dried (Na₂SO₄), filtered, and concentrated to obtain crude Intermediate L, which was purified by column chromatography (100-200 mesh silica gel, 30% EtOAc/petroleum ether) to afford the product (300 mg, 61%) as off white solid. R_{f}=0.4 (20% EtOAc/petroleum ether). ¹H-NMR (400 MHz, DMSO-d₆): 13.38 (br. s, exchanged with D₂O, 1H), 12.32 (br. s, exchanged with D₂O, 1H), 11.23 (br. hump, exchanged with D₂O, 1H), 3.70 (s, 1H), 1.23 (s, 9H). Mass (m/z, APCI, positive mode): 280.1 (M⁺+1).

Preparation of Compound (13)

A mixture of Intermediate L (300 mg, 1.06 mmol) and 3N aqueous HCl (5 mL) was stirred at 60° C. for 1 hour. The reaction mixture was cooled to 0° C., treated with aqueous NH₃, adjusted to pH=8, and the precipitated solid was collected by filtration. This solid was then washed with H₂O and EtOH (2×50 mL) and dried to obtain Compound (13) (100 mg, 47%). R_{f}=0.4 (20% EtOAc/petroleum ether). ¹H-NMR (400 MHz, DMSO-d₆): 10.80 (br. hump, exchanged with D₂O, 1H), 6.56 (br. s, exchanged with D₂O, 2H), 3.62 (s, 1H). Mass (m/z, positive mode): 197.7. LCMS (Column: Zodiacsil 120-5-C-18, (4.6×50) mm; Mobile phase: A: 0.01M aq.HCOONH₄, B: MeOH, T/% B: 0/5, 10/90, 10.1/5; Flow rate: 1.0 mL/min, diluents: Mobile phase+MeOH upon heating): 97.83% at 214 nm, 98.19% at 254 nm; m/z=241.8, Rₜ=3.95 min.

Synthesis of Compound (5)

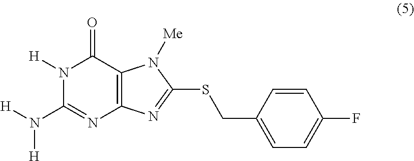

Compound (5) was prepared from Compound (13) according to the following procedure. To a solution of Compound (13) (100 mg, 5.10 mmol) in 0.5N aqueous NaOH (3 mL) was added 4-fluorophenyl methyl bromide (0.12 mL, 10.2 mmol) at 0° C. The reaction was warmed to room temperature (26° C.) and stirred overnight. The reaction mixture was treated with AcOH (5 mL) and stirred for ˜15-20 minutes. The precipitated solid was collected by filtration, washed thoroughly with water (15-20 mL), and dried to obtain crude Compound (5) (20 mg, 13%). R_{f}=0.5 (30% MeOH/CHCl₃/0.2 mL of aqueous NH₃). ¹H-NMR (400 MHz, DMSO-d₆) δ 10.68 (br. s, exchanged with D₂O, 1H), 7.43 (t, J=8.4 Hz, 2H), 7.12 (t, J=8.4 Hz, 2H), 6.09 (br. s. exchanged with D₂O, 2H), 4.43 (s, 2H), 3.62 (s, 3H). Mass (APCI, positive mode, m/z): 306.0. HPLC (Column: Kromasil 100-C-18, (4.6×250) mm, 5μ; Mobile phase: A; 0.01M NH₄OAc, B: MeOH; T/% B: 0/50,5/80/15/80/15.1/80; Flow rate: 1.0 mL/min, Diluents: 200 mL of formic acid in McCN): 94.6% at 214 nm, 94.8% at 254 nm; Rₜ=7.72 min.

Synthesis of Compound (7)

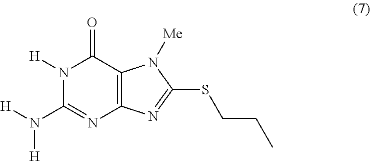

Compound (7) was prepared from Compound (13) according to the following procedure. To a solution of Compound (13) (100 mg, 5.10 mmol) in 0.5N aqueous NaOH (3 mL) was added n-propylbromide (0.12 mL, 10.2 mmol) at 0° C. The reaction was warmed to room temperature (26° C.) and stirred overnight. The reaction mixture was treated with AcOH (5 mL) and stirred for ˜15-20 minutes. The precipitated solid was collected by filtration, washed thoroughly with water (15-20 mL), and dried to obtain crude Compound (7) (15 mg, 12%). R_{f}=0.5 (30% MeOH/CHCl₃/0.2 mL of aqueous NH₃). ¹H-NMR (400 MHz, DMSO-d₆) δ 10.70 (br. hump, exchanged with D₂O, 1H), 6.07 (br. s, exchanged with D₂O, 2H), 3.70 (s, 1H), 3.18 (t, J=6.8 Hz, 2H), 1.68 (quintet, J=6.8 Hz, 2H), 0.97 (t, J=6.8 Hz, 3H). Mass (m/z, APCI, positive mode): 240.1 LCMS (Zodiacsil 120-5-C-18, (4.6×50 mm), mobile phase, A; 0.01M HCOONH₄, B: MeOH; Gradient: T/% B: 0/5, 10/90, 10.1/5, Flow rate: 1.0 mL/min, diluents: aqueous NH₃+MeOH): 93.06% at 214 nm, 92.76 at 254 nm; Rₜ=7.51 min.

Synthesis of Compound (53)

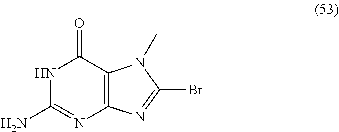

Compound (53) can be prepared from Compound (48) under brominating conditions such as those described in Scheme 14.

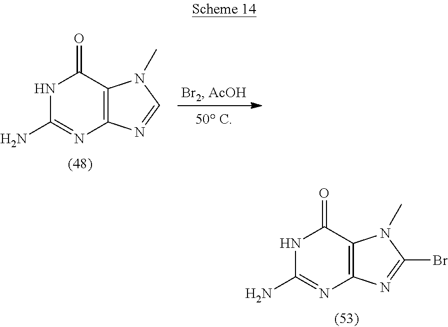

Synthesis of Compounds (2), (4), (12), (28), and (49)

The procedure described in Scheme 3, which uses commercially available Intermediate M as a starting material, has been used to prepare the following compounds where R=Me and where compound (12) can be treated with another electrophile to yield the S-alkyl compound. Additional synthetic information is also provided in Scheme 15.

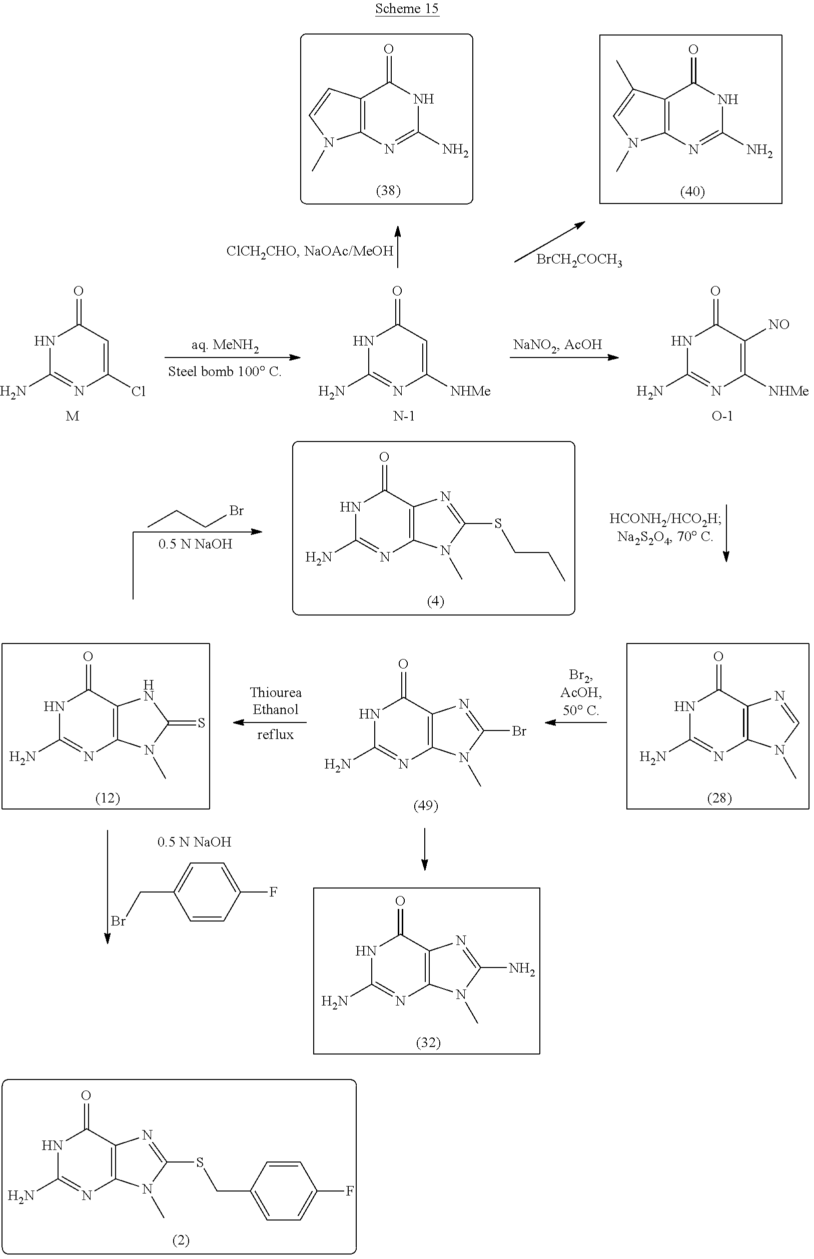

Synthesis of Compound (12)

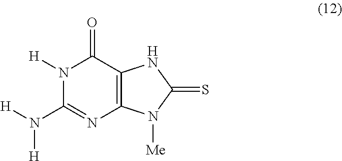

Preparation of Intermediate N-1

To a solution of Intermediate M (5 g, 34.48 mmol) was added 40% aqueous methyl amine solution (50 mL), and the resulting mixture was heated in a steel bomb at 120-130° C. (bath temperature) for 8 hours. The reaction mixture was cooled to room temperature and then concentrated to obtain a crude residue that was washed with EtOH several times to obtain pure Intermediate N-1 (3.5 g, 72%). R_{f}=0.6 (20% MeOH/CHCl₃/0.2 mL of aqueous NH₃). ¹H-NMR (400 MHz, DMSO-d₆) δ 9.66 (br. s, exchanged with D₂O, 1H), 6.25 (br. s, exchanged with D₂O, 1H), 6.10 (br. s, exchanged with D₂O, 2H), 4.36 (s, exchanged with D₂O, 1H), 2.61 (s, 3H). Mass (APCI positive mode, m/z): 141.0 (M⁺+1).

Preparation of Intermediate O-1

To a suspension of Intermediate N-1 (5 g, 35.7 mmol) in a 1:1 mixture of H₂O (50 mL)/AcOH (50 mL) was added NaNO₂ (5 g) in H₂O (50 mL) at 0° C. The reaction was warmed to room temperature and stirred for 1 hour; during this time, formation of an orange red solid was observed. The reaction mixture was then cooled to 0° C., and the precipitated solid was collected by filtration. The solid was washed with H₂O thoroughly and dried to obtain Intermediate 0-1 (2.5 g, 41%), which was used directly in the next step. R_{f}=0.2 (20% MeOH/CHCl₃/0.2 mL of aqueous NH₃). ¹H-NMR (400 MHz, DMSO-d₆) δ 12.28 (br. s, exchanged with D₂O, 1H), 10.95 (br. s, exchanged with D₂O, 1H), 8.30 (br. s, exchanged with D₂O, 1H), 6.92 (br. s, exchanged with D₂O, 2H), 2.88 (d, J=4.8 Hz, addition of D₂O changed to s, 3H). Mass (APCI positive mode, m/z): 170.0 (M⁺+1).

Synthesis of Compound (28)

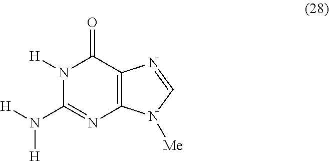

A suspension of Intermediate O-1 (5 g, 29.58 mmol) in a mixture of HCONH₂ (23 mL, 591 mmol) and 90% HCO₂H (16.7 mL, 443 mmol) was heated to 70° C. for 1 hour. At this time, Na₂S₂O₅ (5 g) was added portionwise, and the resulting mixture was heated to 170° C. for 3 hours. The reaction mixture was cooled to room temperature and then poured into ice cold H₂O. The precipitated solid was collected by filtration, washed with cold H₂O, and dried. The crude Compound (28) was further purified by dissolving in 1N aqueous HCl, filtering through a plug of charcoal, neutralizing the filtrate with aqueous NH₃, and then collecting the precipitated solid by filtration to obtain Compound (28) (3.0 g, 61.5%) as an off white solid. R_{f}=0.5 (20% MeOH/CHCl₃/0.2 mL of aqueous NH₃). ¹H-NMR (400 MHz, DMSO-d₆) δ 10.50 (br. s, exchanged with D₂O, 1 H), 7.72 (s, 1H), 6.45 (br. s, exchanged with D₂O, 2H), 3.52 (s, 3H). Mass (m/z, APCI positive mode): 165.9 (M⁺+1). HPLC: (Column: Acquity; UPLC BEH; C-18 (100×2.1 mm) 1.7μ: 0.025% TFA (Aqueous), B: 0.025% TFA (Acetonitrile), T/% B: 0/10, 4/80, 6/80, 6.1/10, Flow rate: 0.3 mL/min, Diluent: A:B (7:3), Rt=0.936 min, 98.33 (215 nm), 99.31 (254 nm), 99.22 (Max plot)).

Synthesis of Compound (49)

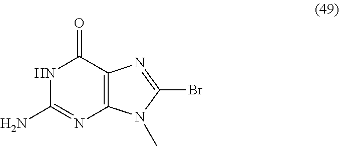

A suspension of Compound (28) (4.0 g, 24.24 mmol) in AcOH (20 mL) was heated to 50° C. for 30 minutes, and Br₂ (1.2 mL, 24.24 mmol) was added. The reaction was stirred at 70° C. for 1.5 hours. The reaction mixture was then cooled to room temperature and poured over ice cold H₂O. The precipitated solid was then collected by filtration, washed with cold H₂O, and dried to obtain compound Compound (49) (5.0 g, 68%) as yellow solid. R_{f}=0.6 (20% MeOH/CHCl₃/0.2 mL of aqueous NH₃). ¹H-NMR (400 MHz, DMSO-d₆) δ 10.68 (br. s, exchanged with D₂O, 1H), 6.52 (br. s, exchanged with D₂O, 2H), 3.47 (s, 3H). Mass (m/z, APCI positive mode): 244.0 (M⁺+1) & 246.0 (M¹+3). HPLC: (Column: Atlantis C-18; (250×4.6 mm); 5μ, Mobile phase: A: 0.1% Formic acid (Aq), B: MeOH, T/% B: 0/20, 10/80, 15/80, 15.1/20, Flow rate: 1.0 mL/min, Diluent: A:B (1:1), Rₜ=7.373, 98.22 (258 nm)).

Preparation of Compound (12)

A solution of Compound (49) (2 g, 8.23 mmol) and thiourea (1.25 g, 16.46 mmol) in EtOH (10 mL) was heated to 80° C. for 4 hours. The reaction mixture was cooled to room temperature and concentrated to obtain Compound (12) as a residue, which was thoroughly washed with H₂O and dried to obtain the product (1.0 g, 62.5%) as off white solid. R_{f}=0.2 (20% MeOH/CHCl₃/0.2 mL of aqueous NH₃). ¹H-NMR (400 MHz, DMSO-d₆) δ 12.73 (br. s, exchanged with D₂O, 1H), 10.97 (br. s, exchanged with D₂O, 1H), 6.167 (br. s, exchanged with D₂O, 2H), 3.38 (s, 3H). Mass (m/z, APCI-ve mode): 196.0 (M⁺−1). HPLC: (Column: Atlantis C-18; (250×4.6 mm); 5μ, Mobile phase: A: 0.01M Ammonium acetate, B: MeOH, T/% B: 0/30, 10/80, 15/80, 15.1/30, Flow rate: 0.8 mL/min, Diluent: A:B (1:1), Rₜ=6.723 min, 97.31 (215 nm), 97.06 (254 nm), 96.63 (Max plot)).

Synthesis of Compound (2)

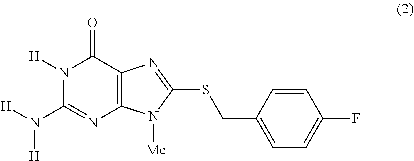

To a solution of Compound (12) (300 mg, 1.52 mmol) in 0.5N aqueous NaOH (5 mL) and THF (1 mL) was added 4-fluorophenyl methyl bromide (0.228 mL, 10.2 mmol) at room temperature (26° C.), and the reaction stirred overnight. The reaction mixture was treated with AcOH (5 mL) and stirred for ˜15-20 minutes. The resulting precipitated solid was collected by filtration, washed thoroughly with water (15-20 mL), and dried to obtain crude Compound (2), which was further purified by preparative HPLC (Column: Zodiac Sil 120-5-C-4, (150×4.6) mm, 5μ: Mobile phase: A: 0.01M NH₄OAc, B: MeOH; Gradient: T/% B 0/10, 10/80, 20/80, 20.1/80, Flow rate: 1 mL/min, Diluents: MeOH) to obtain the purified product (30 mg, 6.4%). R_{f}=0.5 (30% MeOH/CHCl₃/0.2 mL of aqueous NH₃). ¹H-NMR (400 MHz, DMSO-d₆) δ 10.57 (br. s, exchanged with D₂O, 1H), 7.38 (t, J=8.4 Hz, 2H), 7.12 (t, J=8.4 Hz, 2H), 6.48 (br. s, exchanged with D₂O, 2H), 4.43 (s, 2H), 3.30 (s, 3H). Mass (ES positive mode, m/z): 306.41. HPLC (Column: Acquity UPLC BEH C-18, 1.7μ (2.1×50) mm, Mobile phase, A; 0.01% HCOOH, B: 0.01% HCOOH in MeCN; T/% B: 0/15, 3/90, 5/90, 5.1/15; Flow rate: 0.4 mL/min, Diluents: MeCN)): 99.1% at 214 nm, 98.27 at 254 nm, Rₜ=1.48 min.

Synthesis of Compound (4)

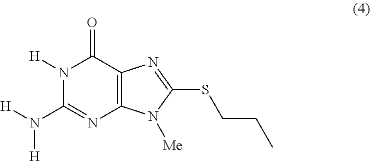

To a solution of Compound (12) (300 mg, 1.53 mmol) in 0.5 N aqueous NaOH (5 mL) was added n-propylbromide (0.2 mL, 2.29 mmol) at room temperature (26° C.), and the reaction stirred overnight. The reaction mixture was treated with AcOH (5 mL) and stirred for ˜15-20 minutes. The precipitated solid was collected by filtration, washed thoroughly with water (15-20 mL), and dried to obtain crude Compound (4) (30 mg, 8.2%). R_{f}=0.5 (30% MeOH/CHCl₃/0.2 mL aqueous NH₃). ¹H NMR (400 MHz, DMSO-d₆) δ 10.51 (br. s, exchanged with D₂O, 1H), 6.44 (br. s, exchanged with D₂O, 2H), 3.10 (t, J=7.2 Hz, 2H), 1.67 (quintet, J=, 7.2 Hz, 2H), 0.97 (t, J=7.2 Hz, 3H). Mass (m/z)=240.1. HPLC (Column: Acquity; UPLC BEH, C-18 (100×2.1) mm 1.7 g; B=Mobile phase A: 0.01M (NH₄)₂CO₃, B: MeCN, T/% B: 0/20, 4/80, 6/80, 6.1/20, Flow rate: 0.3 mL/min, diluents: A:B (7:3)): Rₜ 1.75 min, purity: 87.87% at 214 nm, 89.76% at 254 nm.

Synthesis of Compound (14)

The procedure of Scheme 3 was used to prepare Compound (14).

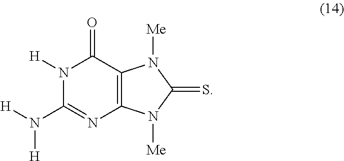

Synthesis of Compounds (11), (50), (51), and (52)

The procedure of Scheme 4 has been used to prepare Compounds (11), (50), (51), and (52).

Synthesis of Compound (50)

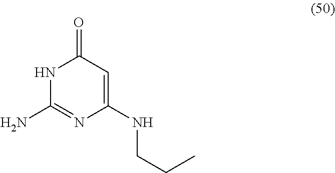

To a solution of Intermediate M (5 g, 34.48 mmol) was added n-propylamine (50 mL) and H₂O (20 mL), and the resulting mixture was heated in a steel bomb at 120-130° C. (bath temperature) for 8 hours. The reaction mixture was cooled to room temperature and concentrated to obtain a crude residue. This residue was washed with EtOH several times to obtain pure Compound (50) as a yellow solid. R_{f}=0.5 (20% MeOH/CHCl₃/0.2 mL of aqueous NH₃). ¹H-NMR (400 MHz, DMSO-d₆) δ 9.62 (s, exchanged with D₂O, 1H), 6.35 (s, exchanged with D₂O, 1H), 6.10 (s, exchanged with D₂O, 1H), 4.49 (s, 1H), 3.34 (br. s, 2H), 1.70-1.60 (m, 2H), 0.85 (t, J=7.6 Hz, 2H). Mass (m/z): 168.8 (M⁺+1). HPLC: (Column: Acquity; UPLC BEH; C-8 (100×2.1 mm) 1.7μ, A: 0.05M Ammonium bicarbonate (Aqueous), B: Acetonitrile, T/% B: 0/20, 4/80, 6/80, 6.1/20, Flow rate: 0.3 mL/min, Diluent: A:B (7:3)), Rₜ=1.194 min, 98.07 (215 nm), 98.51 (254 nm), 96.45 (Max plot).

Preparation of Intermediate N-2

To a suspension of Compound (50) (1.2 g, 6.55 mmol) in a 1:1 mixture of H₂O (12 mL)/AcOH (12 mL) was added NaNO₂ (1.2 g) in H₂O (12 mL) at 0° C. The reaction was warmed to room temperature and stirred for 1 hour; during this time, formation of an orange red solid was observed. The reaction mixture was cooled to 0° C., and the precipitated solid was collected by filtration, washed with H₂O thoroughly, and dried to obtain Intermediate P-2 (0.65 g, 46%). This compound was found to be sufficiently pure to be used in the next step without additional purification. R_{f}=0.2 (20% MeOH/CHCl₃/0.2 mL of aqueous NH₃). ¹H-NMR (400 MHz, DMSO-d₆+D₂O; mixture of isomeric forms is observed, NMR spectrum is clean upon D₂O exchange) δ 3.36 (t, J=8.0 Hz, 2H), 1.53 (quintet, J=7.6 Hz, 2H), 0.89 (t, J=7.6 Hz, 2H). Mass (APCI positive mode, m/z): 198.1 (M⁺+1).

Synthesis of Compound (51)

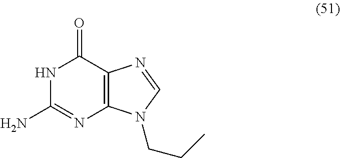

A suspension of Intermediate N-2 (0.6 g, 3.08 mmol) in a mixture of HCONH₂ (2.45 mL, 61.5 mmol) and 90% HCO₂H (1.74 mL, 46.08 mmol) was heated to 70° C. for 1 hour. At this time, Na₂S₂O₅ (0.6 g) was added portionwise, and the resulting mixture was heated to 170° C. for 3 hours. The reaction mixture was cooled to room temperature and poured over ice cold H₂. The precipitated solid was collected by filtration, washed with cold H₂O, and dried. The crude Compound (51) was further purified by dissolving the material in 1N aqueous HCl, filtering through a plug of charcoal, neutralizing the filtrate with aqueous NH₃, and then collecting the precipitated solid by filtration. The desired product (0.35 g) was obtained as an off white solid. R_{f}=0.5 (20% MeOH/CHCl₃/0.2 mL of aqueous NH₃). ¹H-NMR (400 MHz, DMSO-d₆) δ 10.5 (br. s, exchanged with D₂O, 1H), 7.67 (s, 1H), 6.41 (br. s, exchanged with D₂O, 2H), 3.87 (t, J=7.2 Hz, 2H), 1.69 (sextet, J=7.6 Hz, 2H), 0.83 (t, J=7.6 Hz, 2H). Mass (m/z): 193.8 (M⁺+1). LCMS: (Column: Zodiacsil 120-5-C-18-Aq (4.6×50 mm), Mobile phase: A: 0.01M HCOONH₄ (Aq); B: MeOH, T/% B: 0/5, 10/90, 10.1/5, Flow: 1.0 mL/min, Diluent: MeOH+Mobile phase), Rₜ=4.439 min, 99.33 (214 nm), 99.51 (254 nm).

Synthesis of Compound (52)

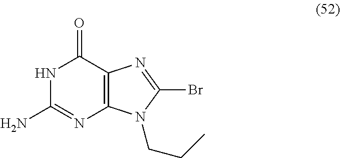

A suspension of Compound (51) (0.35 g, 1.81 mmol) in AcOH (5 mL) was heated to 50° C. for 30 minutes. At this time, Br₂ (1.2 mL, 24.24 mmol) was added, and the reaction stirred at 70° C. for 1.5 hours. The reaction mixture was cooled to room temperature and poured over ice cold H₂O. The precipitated solid was then collected by filtration, washed with cold H₂O, and dried to obtain Compound (52) (0.25 g) as yellow solid. R_{f}=0.5 (20% MeOH/CHCl₃/0.2 mL of aqueous NH₃). ¹H-NMR (400 MHz, DMSO-d₆): 10.66 (s, exchanged with D₂O, 1H), 6.60 (s, exchanged with D₂O, 2H), 4.25 (s, exchanged with D₂O, moisture or unidentified impurity), 3.87 (t, J=7.6 Hz, 2H), 1.70-1.60 (m, 2H), 0.87 (t, J=7.6 Hz, 2H). Mass (APCI+be mode, m/z): 272 (M⁺+1) and 274 (M⁺+3). LCMS: (Column: Zodiacsil 120-5-C-18-Aq (4.6×50 mm), Mobile phase: A: 0.01M HCOONH₄ (Aq); B: MeOH, T/% B: 0/5, 10/90, 10.1/5, Flow: 1.0 mL/min, Diluent: MeOH+ Mobile phase+Heating, Rₜ=6.492 min), 84.56 (214 nm), 87.11 (254 nm).

Synthesis of Compound (11)

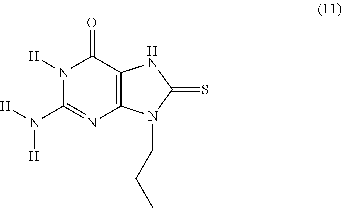

A solution of Compound (52) (0.25 g, 0.922 mmol) and thiourea (0.14 g, 1.845 mmol) in EtOH (10 mL) was heated to 80° C. for 4 hours. The reaction mixture was cooled to room temperature and concentrated to obtain the product as a residue. The residue was thoroughly washed with H₂O and dried to obtain Compound (11) (0.1 g, 48.5%) as an off white solid. R_{f}=0.4 (20% MeOH/CHCl₃/0.2 mL of aqueous NH₃). ¹H-NMR (400 MHz, DMSO-d₆) δ 12.72 (br. s, exchanged with D₂O, 1H), 10.87 (s, exchanged with D₂O, 1H), 6.65 (br. s, exchanged with D₂O, 2H), 3.92 (t, J=8.0 Hz, 2H), 1.69 (quintet, J=7.6 Hz, 2H), 0.86 (t, J=7.6 Hz, 2H). Mass (m/z): 225.8 (M⁺+1). LCMS: (Column: Zodiacsil 120-3-C-18-Aq (4.6×50 mm), Mobile phase: A: 0.01M HCOONH₄ (Aq); B: MeOH, T/% B: 0/5, 10/90, 10.1/5, Flow: 1.0 mL/min, Diluent: MeOH, Rₜ=6.086 min), 96.28% (254 nm).

### Synthesis of Formula (II) Compounds

Synthesis of Compounds (15)-(20)

The procedure described in Scheme 5 was used to prepare Compounds (17) and (18). This procedure is also described in Scheme 16.

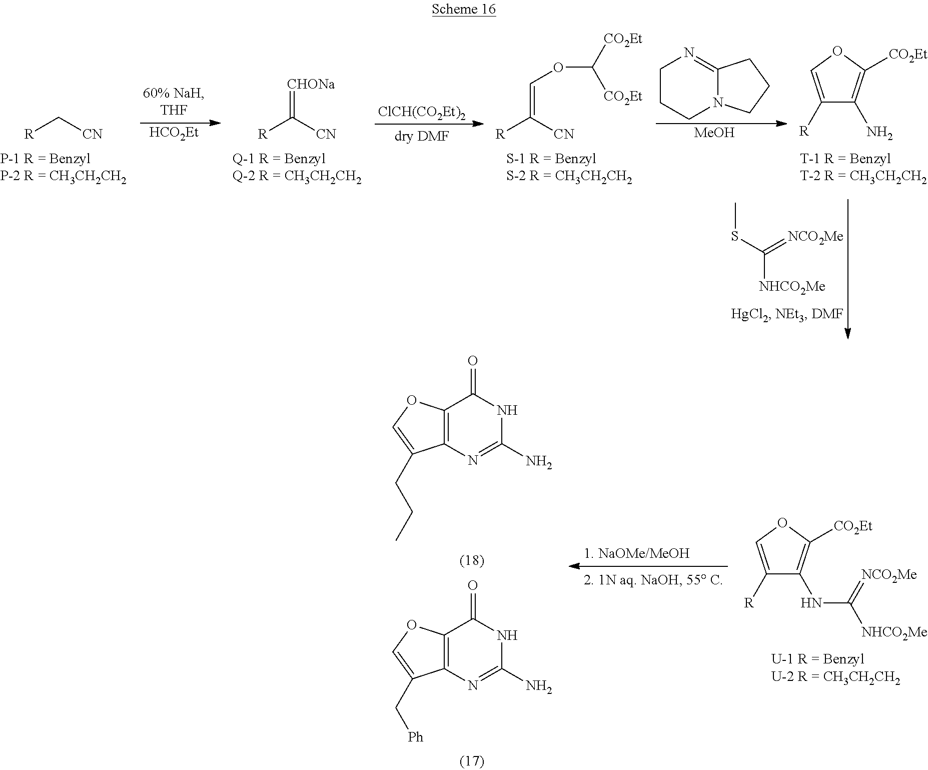

Synthesis of Compound (17)

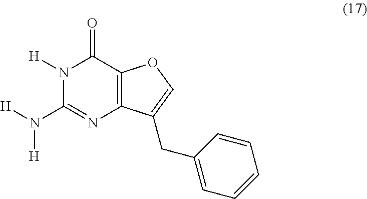

Preparation of Intermediate Q-1

To a solution of compound P-1 (10 g, 76.33 mmol) in THF (125 mL) was added NaH (6.1 g, 152.6 mmol, 60% dispersion in oil) at 0° C. Ethyl formate (12.28 mL, 152.66 mmol) was then added, and an exothermic reaction was observed. The reaction mixture was warmed to room temperature and stirred overnight. Petroleum ether (˜100 mL) was added, and the reaction was stirred for ˜1 hour. The precipitated sodium salt of compound Q-1 was collected by filtration and washed with petroleum ether. The sodium salt of Intermediate Q-1 was neutralized by treating with 1N aqueous HCl. The mixture was extracted with EtOAc, washed with H₂O, brine solution, dried (Na₂SO₄), and evaporated to obtain the purified product. R_{f}=0.4 (20% EtOAc/petroleum ether). ¹H-NMR (400 MHz, CDCl₃) δ 8.50 (s, 1H), 7.50-7.10 (series of m, 5H), 3.35 (s 2H). Mass (APCI positive mode, m/z): 158.1 (M⁺+1).

Preparation of Intermediate S-1

To a solution of Intermediate Q-1 (3.06 g, 19.24 mmol) in DMF (25 mL) was added ethyl 2-chloromalonate (3.11 g, 19.24 mmol) at room temperature (26° C.), and the mixture stirred overnight. To the reaction mixture was then added EtOAc (−200 mL). The resulting mixture was then washed with 1N aqueous HCl (−50 mL), H₂O (2×100 mL), brine solution (50 mL), dried (Na₂SO₄), filtered, and evaporated to obtain crude Intermediate S-1, which was purified by column chromatography (100-200 mesh silica gel, EtOAc/petroleum ether) to obtain the product (1.25 g, 33%). R_{f}=0.4 (20% EtOAc/petroleum ether). ¹H-NMR (400 MHz, CDCl₃) δ 7.50-7.10 (series of m, 5H), 4.93 (s, 1H), 4.30-4.10 (q, J=6.8 Hz, 2H), 3.6 (s, 1H), 1.35 (t, J=6.8 Hz, 3H). Mass (APCI positive mode, m/z): 316 (M⁺+1).

Preparation of Intermediate T-1

To a solution of Intermediate S-1 (2.0 g, 6.30 mmol) in EtOH (25 mL) was added DBN (0.86 mL, 6.94 mmol) at room temperature (26° C.) and stirred at 75-80° C. overnight. The reaction mixture was concentrated to obtain a residue, and EtOAc (˜100 mL) was added. The mixture was then washed with H₂O (50 mL) and brine solution (50 mL), dried (Na₂SO₄), filtered, and evaporated to obtain crude Intermediate T-1, which was purified by column chromatography (100-200 mesh silica gel, 8→10% EtOAc/petroleum ether) to obtain the product (0.35 g, 22.6%). R_{f}=0.65 (20% EtOAc/petroleum ether). ¹H-NMR (400 MHz, CDCl₃) δ 7.50-7.10 (series of m, 6H), 4.30-4.10 (overlapped s and q, J=6.8 Hz, 4H), 3.72 (s, 2H), 1.35 (t, J=6.8 Hz, 3H). Mass (APCI positive mode, m/z): 246.1 (M⁺+1).

### Preparation of Intermediate U-1

A mixture of Intermediate T-1 (200 mg, 0.816 mmol), Et₃N (0.339 mL, 2.44 mmol) and 1,3-dicarbomethoxy-2-methyl-2-thiopseudourea (168 mg, 0.816 mmol) in DMF (8 mL) was added HgCl₂ (220 mg, 0.819 mmol) at room temperature (26° C.) and then stirred overnight. The reaction mixture was filtered, and the filtrate was diluted with H₂O (50 mL) and extracted with EtOAc (˜150 mL). The organic extracts were washed with H₂O (50 mL) and brine solution (50 mL), dried (Na₂SO₄), filtered, and evaporated to obtain crude Intermediate U-1 (125 mg), which was used in the next strep without further purification. R_{f}=0.3 (20% EtOAc/petroleum ether). Mass (APCI positive mode, m/z): 232.1 (M⁺+1).

Preparation of Compound (17)

To a solution of Intermediate U-1 (125 mg, 0.310 mmol) in MeOH (10 mL) was added NaOMe (83 mg, 1.55 mmol) at 0° C. The reaction was warmed to room temperature and stirred overnight. The reaction mixture was concentrated and 1N aqueous NaOH (2.5 mL) was added. The reaction was heated to 60° C. for 30 minutes. At this time, the reaction mixture was evaporated to obtain a crude residue that was purified by column chromatography (100-200 mesh silica gel, MeOH/CHCl₃/aq. NH₃) to obtain Compound (17) (15 mg, 7.6% from Intermediate U-1). R_{f}=0.6 (20% 20% MeOH/CHCl₃/0.1 mL of aqueous NH₃). ¹H-NMR (400 MHz, DMSO-d₆) δ 10.86 (br. s, exchanged with D₂O, 1H), 7.65 (s, 1H), 7.28-7.10 (m, 5H), 6.31 (br. s, exchanged with D₂O, 2H), 3.80 (s, 2H). Mass (m/z, APCI negative scan): 240.1 (M⁺−1). LCMS: (Column: Zodiacsil 120-5-C-18-Aq (4.6×50 mm), Mobile phase: A: 0.01M HCOONH₄ (Aq); B: MeOH, T/% B: 0/5, 10/90, 10.1/5, Flow: 1.0 mL/min, Diluent: McOH+Mobile phase+Heating), Rₜ=7.700 min, 91.45 (214 nm), 83.56 (254 nm).

Synthesis of Compound (18)

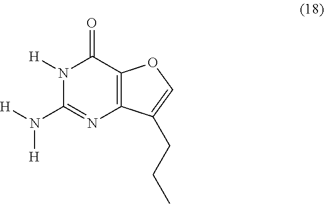

Preparation of Intermediate Q-2

To a solution of Intermediate P-2 (10 g, 120.48 mmol) in THF (125 mL) was added NaH (9.6 g, 240.9 mmol, 60% dispersion in oil) at 0° C. Ethyl formate (19.38 mL, 240.96 mmol) was then slowly added during this time, and an exothermic reaction was observed. The reaction mixture was warmed to room temperature and stirred overnight. Afterwards, petroleum ether (˜100 mL) was added and the mixture stirred for ˜1 hour. The precipitated sodium salt of Intermediate Q-2 was collected by filtration and washed with petroleum ether. The sodium salt of Intermediate Q-2 was neutralized by treating with 1N aqueous HCl. The mixture was then extracted with EtOAc and washed with H₂O and brine solution, dried (Na₂SO₄), and evaporated to obtain Intermediate Q-2. R_{f}=0.55 (20% EtOAc/petroleum ether). Mass (APCI positive mode, m/z): 110.3 (M⁺+1)

### Preparation of Intermediate S-2

To a solution of Intermediate Q-2 (2.0 g, 15.26 mmol) in DMF (20 mL) was added ethyl 2-chloromalonate (2.46 mL, 15.26 mmol) at room temperature (26° C.), and the reaction was stirred overnight. To the reaction mixture was added EtOAc (˜200 mL). The mixture was then washed with 1N aqueous HCl(˜50 mL), H₂O (2×100 mL) and brine solution (50 mL), dried (Na₂SO₄), filtered, and evaporated to obtain crude Intermediate S-2. This material was purified by column chromatography (100-200 mesh silica gel, EtOAc/petroleum ether) to obtain the purified product (3.0 g, impurities were still observed in sample). R_{f}=0.5 (20% EtOAc/petroleum ether). Mass (APCI positive mode, m/z): 268.1 (M⁺+1).

Preparation of Intermediate T-2

To a solution of Intermediate S-2 (3.0 g, 11.15 mmol) in EtOH (30 mL) was added DBN (1.38 mL, 11.13 mmol) at room temperature (26° C.), and the reaction stirred at 75-80° C. overnight. The reaction mixture was concentrated to obtain a residue, to which was added EtOAc (˜100 mL). The resulting mixture was washed with H₂O (50 mL) and brine solution (50 mL), dried (Na₂SO₄), filtered, and evaporated to obtain crude Intermediate T-2, which was purified by column chromatography (100-200 mesh silica gel, 8→10% EtOAc/petroleum ether) to obtain Intermediate T-2 (0.1 g, 2% from Intermediate S-2). R_{f}=0.7 (20% EtOAc/petroleum ether). Mass (APCI positive mode, m/z): 198.1 (M⁺+1).

Preparation of Intermediate U-2

To a mixture of Intermediate T-2 (100 mg, 0.507 mmol), Et₃N (0.21 mL), 1.54 mmol), and 1,3-dicarbomethoxy-2-methyl-2-thiopseudourea (104 mg, 0.504 mmol) in DMF (5 mL) was added HgCl₂ (137 mg, 0.505 mmol) at room temperature (26° C.). The reaction was stirred overnight. The reaction mixture was filtered, and the filtrate was diluted with H₂O (50 mL), extracted with EIOAc (˜150 mL), washed with H₂O (50 mL) and brine solution (50 mL), dried (Na₂SO₄), filtered, and evaporated to obtain crude Intermediate U-2 (150 mg). This material was used in the next step without further purification. R_{f}=0.4 (20% EtOAc/petroleum ether).

Preparation of Compound (18)

To a solution of Intermediate U-2 (150 mg, 0.422 mmol) in MeOH (10 mL) was added NaOMe (114 mg, 2.11 mmol) at 0° C. The mixture was then warmed to room temperature and stirred overnight. The reaction mixture was concentrated, and 1N aqueous NaOH (2.5 mL) was added. The reaction was then heated to 60° C. for 30 minutes. The reaction mixture was evaporated to obtain a crude residue that was purified by column chromatography (100-200 mesh silica gel, 20% MeOH/CHCl₃/0.1 mL of aqueous NH₃) to obtain Compound (18) (10 mg, 10.3% from Intermediate U-2). R_{f}=0.7 (20% 20% MeOH/CHCl₃/0.1 mL aqueous NH₃). ¹H-NMR (400 MHz, DMSO-d₆) δ 10.82 (br. s, exchanged with D₂O, 1H), 7.76 (s, 1H), 6.27 (br. s, exchanged with D₂O, 2H), 2.39 (t, J=7.6 Hz, 2H), 1.54 (sextet, J=7.6 Hz, 2H), 0.90 (t, J=7.2 Hz, 3H). Mass (m/z): 193.8 (M⁺−1). LCMS: (Column: Zodiacsil 120-5-C-18-Aq (4.6×50 mm), Mobile phase: A: 0.01M HCOONH₄ (Aq); B: MeOH, T/% B: 0/30, 5/70, 6/80, 6.1/30, Flow: 1.0 mL/min, Diluent: MeOH), Rₜ=3.806 min, 91.98 (214 nm), 94.08 (254 nm).

The compounds of Table 3 were also prepared according to methods described in Scheme 5.

TABLE 3

| Compound | R |
|---|---|
| 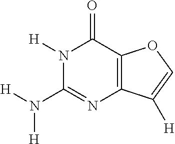 | H |
| (15) | |
| 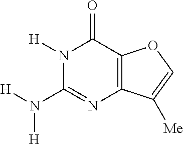 | methyl |
| (16) | |

The procedure described in Scheme 5 was also used to prepare the two compounds of Table 4 by using the indicated starting materials.

TABLE 4

| Compound | Starting Material |
|---|---|
| 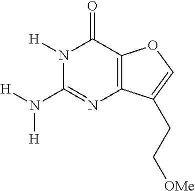 | 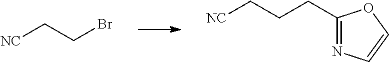 |
| (19) | |
| 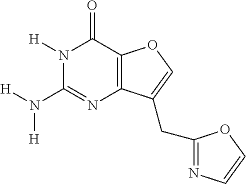 | 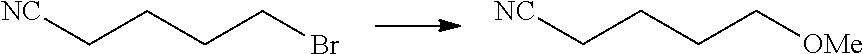 |
| (20) | |

### Synthesis of Formula (III) Compounds

Synthesis of Compounds (21)-(26)

The procedure described in Scheme 6 was used to prepare the oxazolidinone compounds shown in Table 5.

TABLE 5

| Compound | Electrophile R-LG |
|---|---|
| 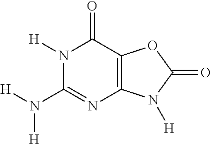 | n/a |
| (21) | |
| 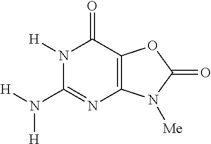 | MeI |
| (22) | |
| 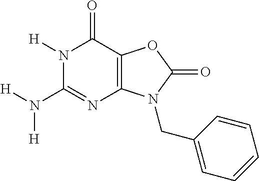 | benzyl bromide |
| (23) | |
| 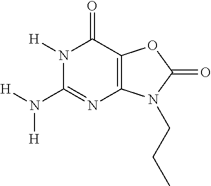 | n-propylbromide |
| (24) | |
| 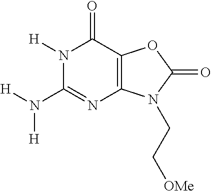 | BrCH₂CH₂OMe |
| (25) | |
| 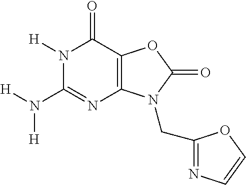 | 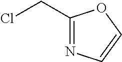 |
| (26) | |

Synthesis of Compound (27)

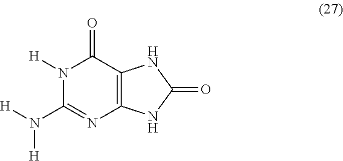

The procedure described in Scheme 17 has been used to prepare Compound (27).

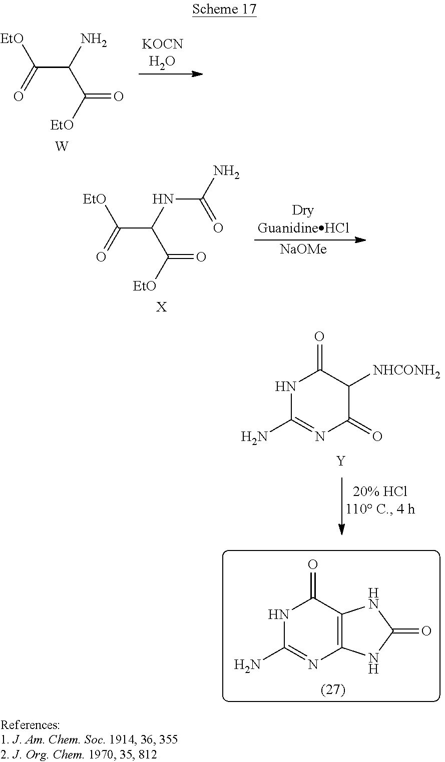

### Preparative Procedures

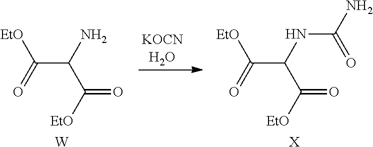

See also: J. Am. Chem. Soc. 36:355, 1914.

To a stirred solution of compound W (1.0 g, 6.36 mmol) in H₂O (10 mL) was added potassium cyanate (0.45 g, 5.67 mmol). The reaction stirred at room temperature for one hour. The product was a white precipitate that separated out from the reaction mixture, which was collected via filtration, washed with ethanol and dried to afford the desired product as an off-white solid. ¹H NMR (300 MHz, DMSO-d₆) δ 6.78 (d, J=7.5 Hz, 1H), 5.90 (s, 2H, NH₂), 4.89 (d, J=7.8 Hz, 1H), 4.22-4.11 (m, 4H), 1.20-1.17 (m, 6H); Mass (M−H): 217 (100).

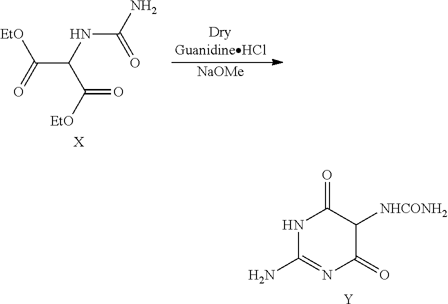

See also: J. Org. Chem. 35:812, 1970.

Compound X (1 g, 4.58 mmol) was dissolved in dry methanol (8 mL; previously distilled from magnesium methoxide). To this solution was added sodium methoxide (488 mg, 9.40 mmol) followed by dry guanidine.HCl (900 mg, 9.40 mmol. The guanidine.HCl reagent had been azeotropically distilled with toluene and dried before use. The reaction mixture was further diluted with methanol (8 mL), and the resulting yellowish mixture was stirred for 3 hours in a N₂ atmosphere under reflux. At this time, most of the solvent was removed under nitrogen flow at 85° C., and the last traces were removed under reduced pressure. The resulting solid cake was washed with cold water (11 mL) and dried over P₂O₅ overnight. The resulting solid was then suspended in water at 5° C. and stirred with 2 mL of 6N aqueous HCl for 30 minutes. The milky white suspension was filtered, and 330 mg of product (40% yield) was obtained for use in the next reaction without further purification.

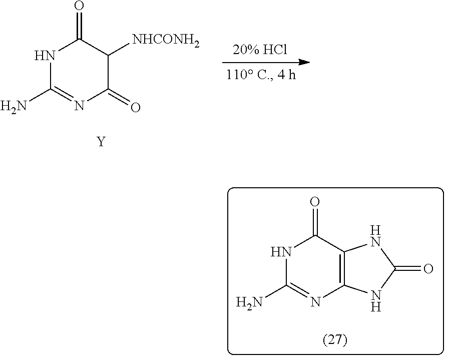

See also: J. Org. Chem. 1970, 35, 812.

Compound Y (330 mg, 1.78 mmol) was heated to reflux in 20% aqueous HCl (100 mL) for 6 hours. At this time, the solvent was evaporated under reduced pressure, and the residue was dissolved in 20% aqueous NaOH (10 mL). The addition of 20% aqueous HCl precipitated the product, which was isolated as a white solid (100 mg, 32.67%). ¹H NMR (300 MHz, DMSO-d₆): δ 10.83 (s, 1H), 10.56-10.54 (br. s, 1H), 10.22 (s, 1H), 6.32 (bs, 2H); Mass (M+H): 167.8 (100).

Synthesis of Compound (32)

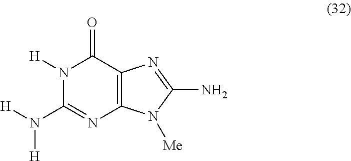

Compound (32) was prepared according to Scheme 15. A mixture of Compound (49) (300 mg, 1.23 mmol) and NH₂NH₂ monohydrate (5 mL) was heated to 130° C. in a sealed tube for 6 hours. Purification of the above reaction mixture was accomplished using preparative HPLC (Column: Zodiacsil 120-5-C-4, (150×4.6) mm, 5μ: Mobile phase: A: 0.01M NH₄OAc, B: MeOH; Gradient: T/% B 0/20, 5/20, 10/80, 10.1/20; Flow rate: 1 mL/min; Diluents: MeOH) to obtain Compound(32) (2 mg, 0.9%). ¹H-NMR (400 MHz, DMSO-d₆) δ 10.5 (br. s, exchanged with D₂O, 1H), 6.54 (br. s, exchanged with D₂O, 1H), 6.30 and 6.27 (2 overlapped br. s, exchanged with D₂O, 3H), 3.28 (s, 3H). Mass (m/z): 180.7 (M++1). LCMS: (Column: Zodiacsil 120-5-C-18-Aq (4.6×50 mm), Mobile phase: A: 0.01M HCOONH₄ (Aq); B: MeOH, T/% B: 0/5, 10/90, 10.1/5, Flow: 1.0 mL/min, Diluent: MeOH+Mobile phase), Rₜ=2.256 min, 90.40 (214 nm), 92.63 (254 nm).

Synthesis of Compounds (28)-(30)

The method used to prepare Compound (28) is described in the synthetic protocol provided for Compound (49) (vide supra; see also Scheme 15).

The method used to prepare Compound (29) is included with the synthetic protocols provided for Compounds (37) and (39) (vide infra; see also Scheme 18).

The procedure described in Scheme 9 was used to prepare Compound (30),

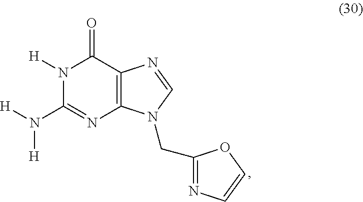

(e.g., J. Med. Chem. 36:3431, 1993 or J. Med. Chem. 28:1870, 1985).

### Synthesis of Formula (IV) Compounds

Synthesis of Compound (38)

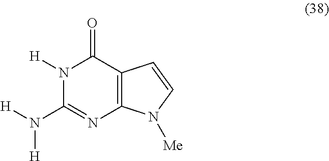

Compound (38) was prepared according to Scheme 15. Intermediate N-1 (0.5 g, 3.57 mmol) was added to a solution of NH₄OAc (1.1 g, 14.28 mmol, 4 equivalents) in H₂O (30 mL). The reaction was then heated to 35-40° C. for ˜15 minutes. A solution of 50% ClCH₂CHO (1.4 mL, 8.91 mmol) was added, and the reaction stirred for 2 hours at 35-40° C. The reaction mixture was filtered to remove undissolved material, and the filtrate evaporated to obtain a residue. This material was washed with cold H₂O and dried to obtain Compound (38) (35 mg, 6%) as an off white solid, along with 100 mg of additional Compound (38). R_{f}=0.5 (30% MeOH/CHCl₃/0.2 mL aqueous NH₃). ¹H-NMR (400 MHz, DMSO-d₆) δ 10.24 (br. s, exchanged with D₂O, 1H), 6.67 (d, J=2.8 Hz, 1H), 6.19 (br. s, exchanged with D₂O, 3H, upon addition of D₂O overlapped signal appeared at 6.23 (d, J=2.8 Hz, 1H)), 3.33 (s, 3H). LCMS: (m/z, positive mode): 164.8 (M⁺+1). (Column: Zodiacsil 120-5-C-18-Aq (4.6×50 mm), Mobile phase: A: 0.01M HCOONH₄ (Aq); B: MeOH, T/% B: 0/5, 10/90, 10.1/5, Flow: 1.0 mL/min, Diluent: MeOH), Rₜ=4.002 min, 92.14 (214 nm), 96.31 (254 nm).

Synthesis of Compound (40)

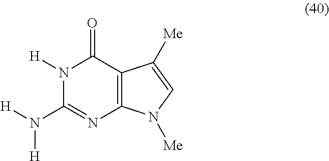

Compound (40) was prepared according to Scheme 15. Intermediate N-1 (0.2 g, 1.42 mmol) was added to a solution of NaOAc (0.194 g, 1.428 mmol) in H₂O (30 mL). The reaction was heated to 65° C. for ˜15 min, and then BrCH₂COCH₃ (0.5 mL, 8.91 mmol) was added. The mixture then stirred for 2 hours at 65° C. The reaction mixture was evaporated to obtain a residue that was purified by column chromatography over silica gel (100-200 mesh, 20% MeOH/CHCl₃/0.2 mL of aqueous NH₃) to obtain Compound (40) (3 mg, ˜1.7%) as an off white solid, along with 10 mg of additional, slightly impure Compound (40). R_{f}=0.7 (30% MeOH/CHCl₃/0.2 mL of aqueous NH₃). ¹H-NMR (400 MHz, DMSO-d₆) δ 10.17 (br. s, exchanged with D₂O, 1H), 6.10 (br. s, exchanged with D₂O, 2H), 5.94 (s, 1H), 3.40 (s, 3H), 2.19 (s, 3H). Mass (m/z, APCI positive mode): 179.0 (M⁺+1). LCMS: (Column: Zodiacsil 120-5-C-18-Aq (4.6×50 mm), Mobile phase: A: 0.01M HCOONH₄ (Aq); B: MeOH, T/% B: 0/5, 10/90, 10.1/5, Flow: 1.0 mL/min, Diluent: MeOH+Mobile phase), Rₜ=5.098 min, 95.09 (214 nm), 93.59 (254 nm).

Synthesis of Compounds (29), (37), and (39)

Compounds (29), (37), and (39) were also synthesized according to the procedure shown in Scheme 18.

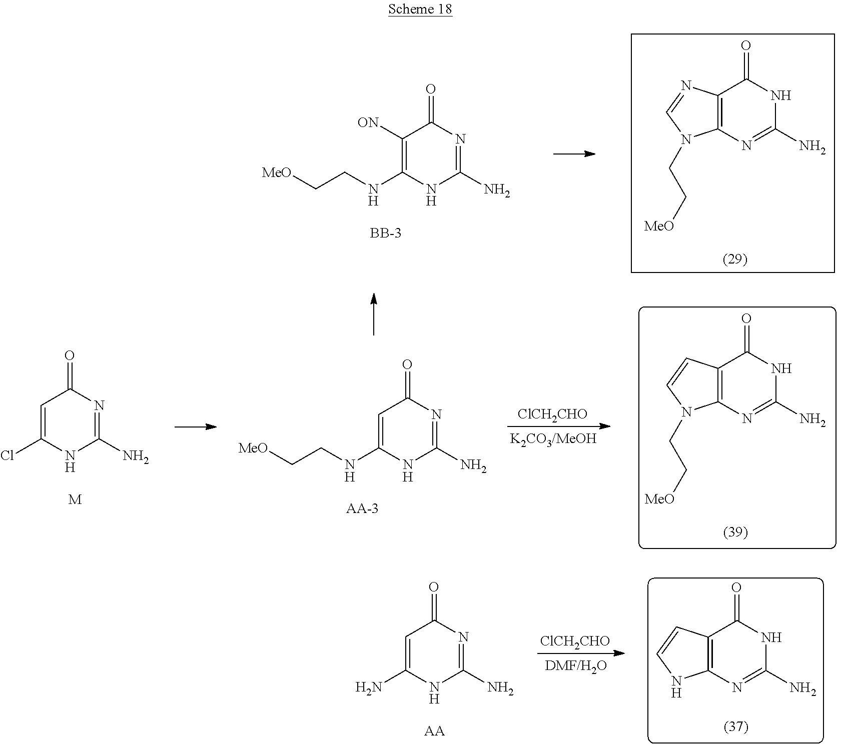

Preparation of Intermediate AA-3

To a solution of Intermediate M (5 g, 34.48 mmol) was added methoxy methylamine (50 mL) and H₂O (20 mL), and the resulting mixture was heated in a steel bomb at 120-130° C. (bath temperature) for 8 hours. The reaction mixture was cooled to room temperature, and concentrated to obtain crude residue that was washed with EtOH several times to obtain pure Intermediate AA-3 (3.0 g, 46%) as a yellow solid. R_{f}=0.6 (20% MeOH/CHCl₃/0.2 mL aqueous NH₃). ¹H-NMR (400 MHz, DMSO-d₆) δ 9.65 (br. s, exchanged with D₂O, 1H), 6.20 (br. s, exchanged with D₂O, 1H), 6.14 (br. s, exchanged with D₂O, 2H), 4.4 (s, 1H), 3.37 (t, J=5.6 Hz, 2H), 3.32 (overlapped s and t, 3H). Mass (APCI positive mode, m/z): 184.9 (M⁺+1).

Synthesis of Compound (29)

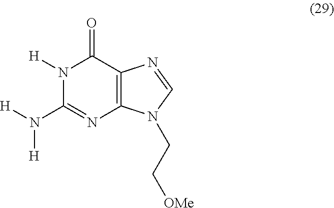

Preparation of Intermediate BB-3

To a suspension of Intermediate AA-3 (2 g, 10.86 mmol) in a 1:1 mixture of H₂O (20 mL)/AcOH (20 mL) was added NaNO₂ (2 g) in H₂O (20 mL) at 0° C. The reaction was warmed to room temperature and stirred for 1 hour. During this time, the formation of an orange red solid was observed. The reaction mixture was cooled to 0° C., and the precipitated solid was collected by filtration, washed with H₂O, and thoroughly dried to obtain Intermediate BB-3 (0.95 g, 41%) that was found to be sufficiently pure for use in the next step. R_{f}=0.2 (20% MeOH/CHCl₃/0.2 mL aqueous NH₃). ¹H-NMR (400 MHz, DMSO-d₆) δ 12.50 (br. s, exchanged with D₂O, 1H), 10.80 (br. s, exchanged with D₂O, 1H), 8.25 (br. s, exchanged with D₂O, 1H), 6.89 (br. s, exchanged with D₂O, 1H), 3.56 (q, J=5.6 Hz, addition of D₂O→t, J=5.6 Hz, 2H), 3.46 (t, J=5.6 Hz, 2H), 3.31 (s, 3H). Mass (APCI positive mode, m/z): 214.1 (M⁺+1).

Preparation of Compound (29)

A suspension of Intermediate BB-3 (0.95 g, 4.50 mmol) in a mixture of HCONH₂ (3.58 mL, 90 mmol) and 90% HCO₂H (2.55 mL, 67.52 mmol) was heated to 70° C. for 1 hour. At this time, Na₂S₂O₅ (0.95 g) was added in portions and the resulting mixture was heated to 170° C. for 3 hours. The reaction mixture was cooled to room temperature and poured over ice cold H₂O. The precipitated solid was collected by filtration, washed with cold H₂O, and dried. The crude Compound (29) was further purified by dissolving in 1N aqueous HCl, filtering through a plug of charcoal, neutralizing the filtrate with aqueous NH₃, and then collecting the precipitated solid by filtration to obtain the purified product (0.3 g, 85%, HPLC purity 85%) as off white solid. R_{f}=0.5 (20% MeOH/CHCl₃/0.2 mL of aqueous NH₃).

Purification of Compound (29) (0.1 g, 85%, HPLC purity 85%) was accomplished by preparative HPLC (Column: Kromasil C-8, 4.6×250 mm, 5μ; Mobile phase: A: 0.01M aqueous NH₄OAc, B: MeOH; T/% B 0/20,3/20, 20/80, 20.1/20; Flow rate: 0.8 mL/min, Diluents: 1:1 Mobile phase). Following purification, 40 mg of Compound (29) was obtained. ¹H-NMR (400 MHz, DMSO-d₆) δ 10.50 (br. s, exchanged with D₂O, 1H), 7.62 (s, 1H), 6.43 (br. s, exchanged with D₂O, 2H), 4.08 (t, J=5.6 Hz, 2H), 3.61 (t, J=5.6 Hz, 2H), 3.23 (s, 3H). Mass (m/z): 237.8 (M⁺+1). LCMS: (Column: Zodiacsil 120-5-C-18-Aq (4.6×50 mm), Mobile phase: A: 0.01M HCOONH₄ (Aq); B: MeOH, T/% B: 0/5, 10/90, 10.1/5, Flow: 1.0 mL/min, Diluent: MeOH+Mobile phase), Rₜ=3.385 min, 98.82 (214 nm), 99.17 (254 nm).

Synthesis of Compound (39)

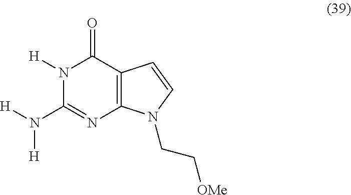

Intermediate AA-3 (0.5 g, 10.27 mmol) was added to a solution of NaOAc (0.369 g, 2.71 mmol, 1.2 eq.) in H₂O (15 mL), heated to 65° C. for ˜15 minutes. A solution of 50% aqueous ClCH₂CHO (0.5 mL) was then added, and the mixture stirred for 2 hours at 65° C. The reaction mixture was filtered to remove undissolved material, and the filtrate was stored at 0° C. overnight. The precipitated solid was collected by filtration, washed with cold H₂O, and dried to obtain crude Compound (39). This material was purified by column chromatography (100-200 mesh silica gel, 20% MeOH/CHCl₃/0.2 mL of aqueous NH₃) to obtain the purified product (120 mg, 20%) as pink solid. R_{f}=0.7 (30% MeOH/CHCl₃/0.2 mL of aqueous NH₃). Mass M/z 151 (M⁺+1). ¹H-NMR (400 MHz, DMSO-d₆) δ 10.23 (br. s, exchanged with D₂O, 1H), 6.69 (d, J=3.6 Hz, 1H), 6.19 (d, J=3.6 Hz, 1H), 6.17 (br. s, exchanged with D₂O, 2H), 4.05 (t, J=5.6 Hz, 2H), 3.58 (t, J=5.6 Hz, 2H), 3.22 (s, 3H). Mass (m/z): 208.8 (M⁺+1). LCMS: (Column: Zodiacsil 120-5-C-18-Aq (4.6×50 mm), Mobile phase: A: 0.01M HCOONH₄ (Aq); B: MeOH, T/% B: 0/5, 10/90, 10.1/5, Flow: 1.0 mL/min, Diluent: MeOH), Rₜ=4.583 min, 94.98 (214 nm), 93.91 (254 nm).

Synthesis of Compound (37)

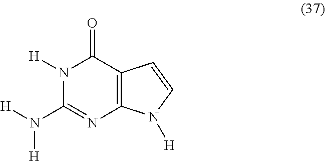

Intermediate AA (0.5 g, 3.96 mmol) was added to a solution of NaOAc (0.647 g, 4.76 mmol, 1.2 eq.) in H₂O (15 mL). The reaction was heated to 60° C. for ˜15 minutes, and a solution of 50% aqueous ClCH₂CHO (0.5 mL) was then added. The reaction stirred for 2 hours at 60° C. The reaction mixture was filtered to remove undissolved material, and the filtrate stored at 0° C. overnight. The resulting precipitated solid was collected by filtration, washed with cold H₂O, and dried to obtain Compound (37) (120 mg, 20%) as a pink solid. R_{f}=0.7 (30% MeOH/CHCl₃/0.2 mL of aqueous NH₃). ¹H-NMR (400 MHz, DMSO-d₆) δ 10.95 (br. s, exchanged with D₂O, 1H), 10.20 (s, exchanged with D₂O, 1H), 6.60 (dd, J=3.2, 2.0 Hz, 1H), 6.18 (dd, J=3.2, 2.0 Hz, 1H), 6.03 (br. s, exchanged with D₂O, 2H). Mass (m/z): 150.8 (M⁺+1). LCMS: (Column: Zodiacsil 120-5-C-18-Aq (4.6×50 mm), Mobile phase: A: 0.01M HCOONH₄ (Aq); B: MeOH, T/% B: 0/5, 10/90, 10.1/5, Flow: 1.0 mL/min, Diluent: MeOH), Rₜ=2.599 min, 99.16 (214 nm), 99.10 (254 nm).

Synthesis of Compound (33)

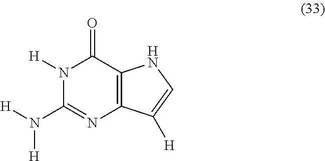

Compound (33) was prepared according to Scheme 19.

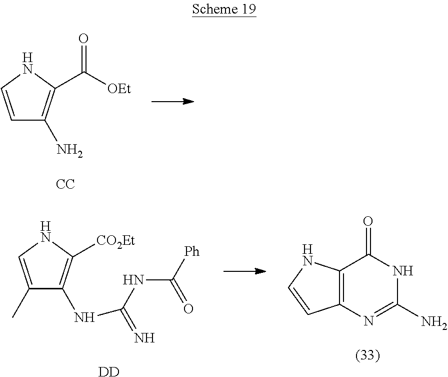

Preparation of Intermediate DD

To a mixture of Intermediate CC (200 mg, 1.049 mmol), Et₃N (0.436 mL, 3.14 mmol), and 1,3-dicarbomethoxy-2-methyl-2-thiopseudourea (216 mg, 1.049 mmol) in DMF (8 mL) was added HgCl₂ (284 mg, 1.049 mmol) at room temperature (26° C.). The mixture was stirred overnight. The reaction mixture was then filtered, and the filtrate was diluted with H₂O (50 mL), extracted with EtOAc (˜150 mL), washed with H₂O (50 mL) and brine solution (50 mL), dried (Na₂SO₄), filtered, and evaporated to obtain crude Intermediate DD (150 mg). This material was used in the next step without any further characterization and purification. R_{f}=0.3 (20% EtOAc/petroleum ether). Mass: (m/z=313.1).

Preparation of Compound (33)

To a solution of Intermediate DD (150 mg, 0.48 mmol) in MeOH (10 mL) was added NaOMe (129 mg, 2.40 mmol) at 0° C. The reaction was warmed to room temperature and stirred overnight. The reaction mixture was concentrated, and 1N aqueous NaOH (2.5 mL) was added. The reaction was heated to 60° C. for 30 minutes. The reaction mixture was evaporated to obtain a crude residue that was purified by column chromatography (100-200 mesh silica gel, 20% MeOH/CHCl₃/aq. NH₃) to obtain Compound (33) (15 mg, 9.6%). R_{f}=0.6 (20% MeOH/CHCl₃/0.1 mL aqueous NH₃). ¹H-NMR (400 MHz, DMSO-d₆) δ 11.36 and 11.20 (2 overlapped br. s, exchanged with D₂O, 2H), 7.05 (s, 1H), 6.05 (br. s, exchanged with D₂O, 2H), 5.88 (d, J=2.4 Hz, 1H). Mass (m/z): 150.7 (M⁺+1). LCMS: (Column: Zodiacsil 120-5-C-18-Aq (4.6×50 mm), Mobile phase: A: 0.01M HCOONH₄ (Aq); B: MeOH, T/% B: 0/5, 10/90, 10.1/5, Flow: 1.0 mL/min, Diluent: MeOH), Rₜ=2.379 min, 97.08 (214 nm), 98.26 (254 nm).

Synthesis of Compounds (34), (35), and (36)

Compounds (34), (35), and (36), which are shown in Table 6, were synthesized according to the procedure described in Scheme 10.

TABLE 6

| Compound | R | R′ |
|---|---|---|
| 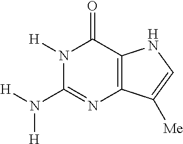 | H | CH₃ |
| (34) | | |
| 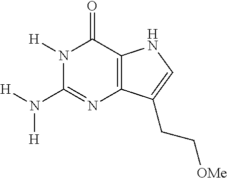 | H | CH₂CH₂OCH₃ |
| (35) | | |
| 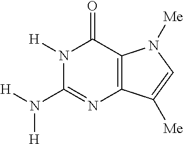 | CH₃ | CH₃ |
| (36) | | |

Synthesis of Compound (42)

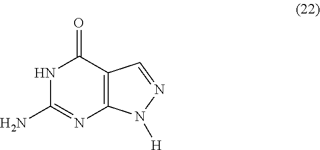

The synthesis of Compound (42) was accomplished using the three step procedure described in Scheme 20.

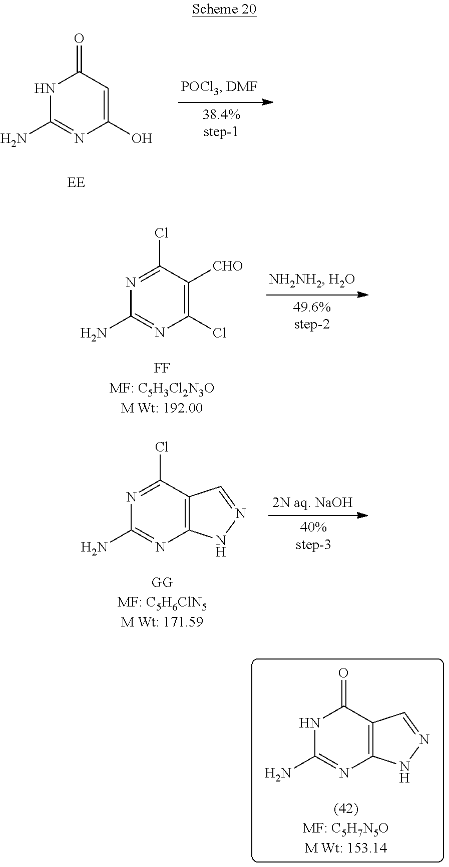

### Step-1

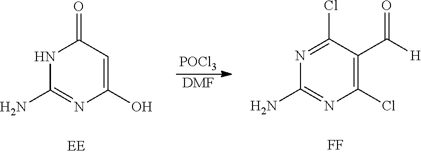

Reference: Helv. Chim. Acta. 69:1602, 1986.

To an ice-cold stirred solution of POCl₃ (9.72 g, 63.38 mmol) was added dry DMF dropwise over a period of 5 minutes. The ice-bath was removed, and Intermediate EE (1.0 g, 7.87 mmol) was added in small portions under stirring and N₂ atmosphere. After the exothermic reaction ceased, the reaction mixture was brought up to 100° C. and stirred for 1.5 hours. The solution was cooled to room temperature, reduced to half the volume under reduced pressure, poured into ice-water (15 mL), and warmed to 50° C. for 2 hours. A pale yellow precipitate then separated out, and the precipitate was filtered, washed with water (˜10 mL) and acetone (˜10 mL), and then dried. The Intermediate FF (580 mg, 38.4%) was obtained as pale yellow solid. ¹H NMR (300 MHz, DMSO-d₆) δ 10.05 (s, CHO), 8.49 (s, 2H, D₂O exchangeable).

### Step-2

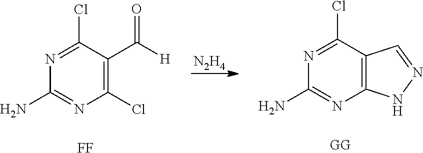

Reference: Helv. Chim. Acta. 69:1602, 1986.

At 50° C., a solution of Intermediate FF (320 mg, 1.66 mmol) in 3:1 THF/H₂O (7 mL) was treated with hydrazine hydrate (170 mg, 3.33 mmol) in H₂O (1.7 mL). The mixture stirred for 20 minutes when a yellowish precipitate was formed. The crude reaction material was poured into ice-cold H₂O (8 mL) and the solvent was reduced to facilitate complete precipitation. The resulting solid was filtered and washed with acetone to yield Intermediate GG as a yellow solid. ¹H NMR (300 MHz, DMSO-d₆): δ 13.25 (s, 1H), 7.95 (s, 1H), 7.15 (bs, 214).

### Step-3

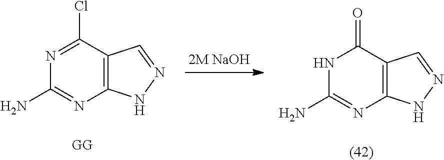

### Reference: Tetrahedron 48:8089, 1992.

A solution of Intermediate GG (250 mg, 1.47 mmol) in 2N aqueous NaOH (4 mL) was refluxed for 20 hours. The solution was cooled to room temperature and acidified (pH 5) with concentrated HCl. The precipitated solid was collected by filtration, washed with water, and dried. The solid was again boiled in 30% acetic acid, filtered, washed with water, and dried to give Compound (42) as a yellow solid. ¹H NMR (300 MHz, DMSO-d₆): δ 12.76 (bs, 1H), 10.45 (s, 1H), 7.76 (s, 1H), 6.49 (bs, 2H); IR (KBr Pellet) cm⁻¹: 3348, 1719, 1671, cm⁻¹; Mass (M+H)=152.

### Synthesis of Compound (43)

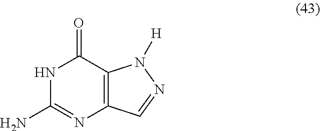

The synthesis of Compound (43) was accomplished using the procedure described in Scheme 21.

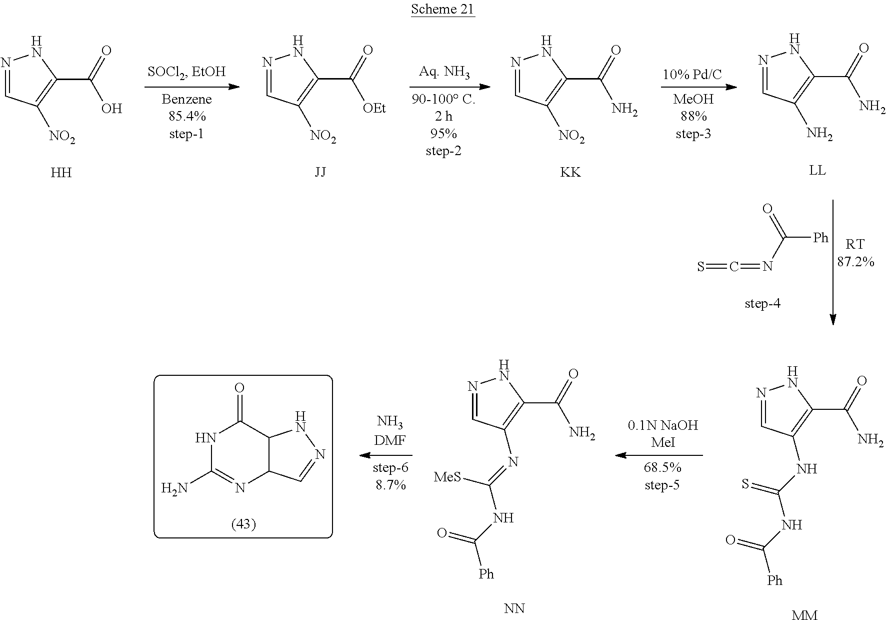

Step-1

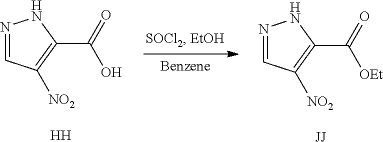

To an ice-cold stirred solution of intermediate HH (1.0 g, 6.36 mmol) in ethanol (17 mL) was added SOCl₂ (0.83 g, 7.00 mmol) dropwise. The reaction was then stirred at room temperature for 10 hours. At this time, excess SOCl₂ and ethanol were evaporated under reduced pressure to yield Intermediate JJ as a solid (1 g, 85.4%). ¹H NMR (300 MHz, CDCl₃): δ 8.48 (s, 1H), 4.51 (q, J=7.17 Hz, 2H), 1.44 (t, J=7.3 Hz, 3H); Mass (M−H): 184 (100).

Step-2

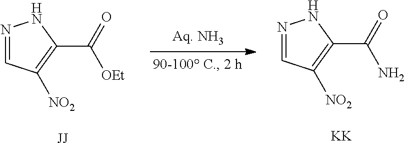

Reference: J. Am. Chem. Soc. 1956, 784, 2418.

Intermediate JJ (1 g, 5.40 mmol) was added to 20 mL of concentrated ammonium hydroxide solution (20 mL) and the mixture was heated at 100° C. for 4 hours. The solvent was evaporated under reduced pressure, and toluene (2×10 mL) was added to the residue. The toluene was azeotropically distilled to yield Intermediate KK (800 mg, 95%), which was used for the next step without further purification. Mass (M−H): 155 (100).

Step-3

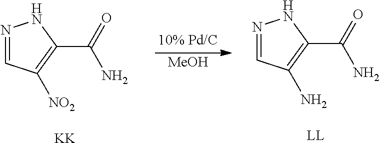

Reference: J. Am. Chem. Soc. 1956, 784, 2418.

To a solution of Intermediate KK (5.0 g, 32.04 mmol) in methanol (95 mL) in a stainless steel Parr hydrogenation flask was added a slurry of 10% Pd/C (500 mg) in methanol (5 mL). The resulting mixture was shaken at 20 psi at room temperature for a period of 2 hours. The catalyst was filtered over a Celite bed, which was then washed with MeOH. The combined filtrates were evaporated under reduced pressure to yield Intermediate LL (3.0 g, 88%), which was used for the next step without further purification. Mass (M+H): 127 (100).

Step-4

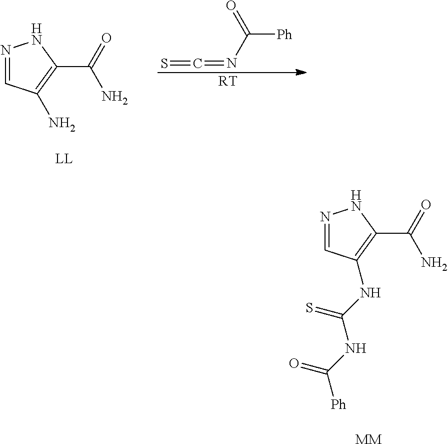

Reference: J. Am. Chem. Soc. 1982, 104, 1073.

Intermediate LL (4.0 g, 31.74 mmol) was dissolved by warming in water (200 mL). A mixture of benzoyl isothiocyanate (5.7 g, 34.92 mmol) and ethanol (200 mL) was added slowly to the rapidly stirred solution of compound J. The mixture was stirred for 2 hours, during which time the sides of the flask were frequently scraped clean of adhering solid. At this time, the solid was collected, washed with hot ethanol (50 mL), and dried to yield Intermediate MM as a pale yellow solid (8 g, 87.2%). ¹H NMR (300 MHz, DMSO-d₆): δ 13.68 (s, 1H), 13.3 (s, 1H), 11.4 (s, 1H), 9.09 (s, 1H), 7.96 (d, J=7.5 Hz, 2H), 7.71 (bs, 1H), 7.66-7.63 (m, 1H), 7.55-7.51 (m, 2H), 7.41 (bs, 1H), Mass (M+H): 290 (100).

Step-5

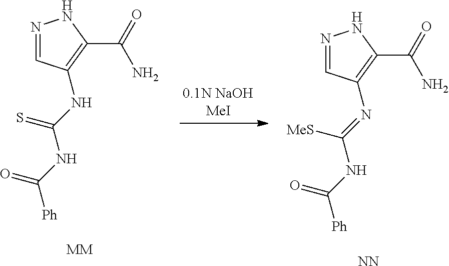

Reference: J. Am. Chem. Soc. 1982, 104, 1073.

Intermediate MM (7.0 g, 24.19 mmol) was dissolved in 0.1 N aqueous NaOH (1050 mL), followed by the addition of MeI (2.1 mL, 33.87 mmol). The reaction was stirred at room temperature for two hours. At this time, the reaction mixture was acidified using acetic acid (pH˜6), and the precipitated solid was filtered, washed with water (50 mL), acetone (50 mL), and dried to yield Intermediate NN (5 g, 68.5%) obtained as off white solid. ¹H NMR (300 MHz, DMSOd₆): δ 13.40 (bs, 1H), 10.8 (bs, 1H), 8.38 (bs, 1H), 8.12-8.10 (m, 2H), 7.94 (bs, 1H), 7.66-7.50 (m, 4H), 2.58 (s, 3H), Mass (M−H): 302 (100).

Step-6

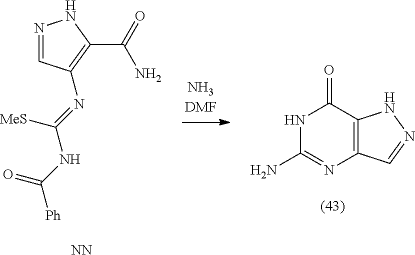

Reference: J. Am. Chem. Soc. 1982, 104, 1073.

A mixture of Intermediate NN (600 mg, 1.98 mmol) and N,N-dimethylformamide (20 mL), which had been previously saturated with ammonia at 0° C., was placed in a sealed tube and heated at 125-130° C. for 2.5 hours. The reaction mixture was cooled, and the solvent was evaporated to dryness. The solid was collected by filtration, and the solid was then washed with water and dried. The solid was stirred and heated at reflux in a 1N NaOH (7 mL) solution for 3.5 hours. The solution was acidified (pH˜6) with concentrated HCl, and the mixture was allowed to stand at 5° C. for ˜12 hours. The solid which had separated was collected by filtration, washed with water (˜10 mL) and acetone (5 mL), and dried. The dry solid was then extracted with boiling ethanol (3×5 mL), and the ethanol insoluble solid was recrystallized from water (5 mL) to obtain (104 mg) of crude Compound (43). Pure Compound (43) was obtained as an off-white solid (26 mg, 8.7% yield) by reprecipitation (at pH ˜6.0) from hot sodium hydroxide with diluted HCl, recrystallization from water, and then purification using preparative HPLC. ¹H NMR (300 MHz, DMSO-d₆): δ 8.29 (bs, 2H), 7.57 (s, 1H), 6.10 (s, 2H), IR (KBr): 3399, 3338, 1695, 1648 cm⁻¹; Mass (M−H):150 (100).

### Screening Conditions for Identifying GCH-1 Inhibition

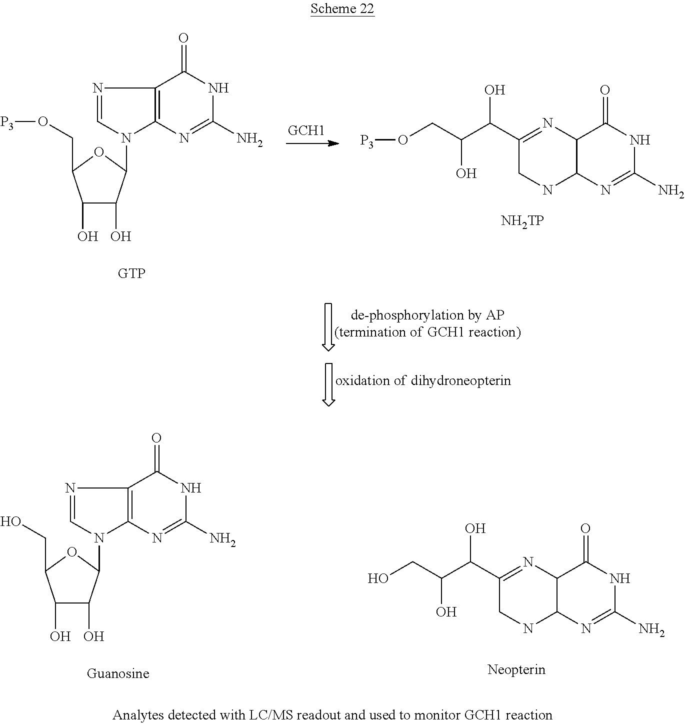

Scheme 22 shows the LC/MS assay used to monitor substrate consumption and product formation. This method is based on transformations known in the art (see, for example, Xie et al., J. Biol. Chem. 273(33):21091-21098, 1998). The LC-MS assay is carried out by the following steps.

(1) GCH-1 reaction
2 μM GCH-1, 120 μM GTP, 1 hour incubation at 37° C.
   (2) Termination of GCH-1 reaction by de-phosphorylation
The reaction is stopped by the addition of excess alkaline phosphatase (2 units/sample)
1 hour incubation at 37° C.
   (3) Oxidation of 7,8-dihydroneopterin to get a stabile final product
Oxidation with 0.1 M 12/KI for 1 hour at 37° C. resulting in oxidation of 7,8-dihydroneopterin to neopterin
Termination of the oxidation reaction by addition of ascorbic acid

### (4) LC/MS Readout

Sample analysis using highly specific LC/MS methods to detect guanosine and neopterin in parallel
Data analysis and automated CRC fitting to get IC₅₀ values

The assay sensitivity was validated and confirmed using the known GCH-1 inhibitors 8-mercaptoguanine and 8-azaguanine (Table 7). The obtained IC₅₀ values were well in the expected potency range (Yoneyama et al., Arch. Biochem. Biophys. 388:67-73, 2001), and the LC/MS based assay was even more sensitive when compared to other GCH-1 screening assays (for example, chromogenic GCH-1 screening assays).

TABLE 7

| | IC₅₀ [μM] | |
|---|---|---|
| Inhibitor | LC/MS assay | IC₅₀ [μM] Literature |
| 8- | 5 | 24 |
| mercaptoguanine | | |
| 8-azaguanine | 5 | 21 |

Exemplary results obtained from the assay are shown in Table 8.

TABLE 8

| No. | Structure | IC₅₀ (μM) |
|---|---|---|
| (3) | 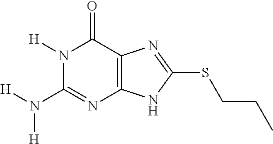 | 14 |
| (4) | 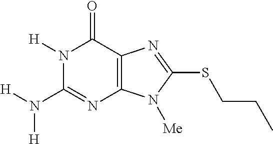 | 85 |
| (9) | 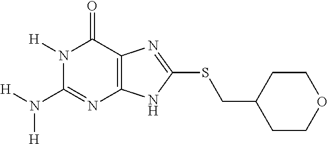 | 39 |
| (6) | 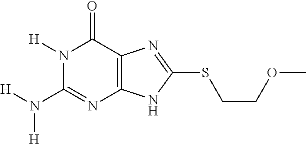 | 25 |
| (13) | 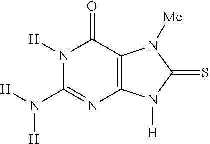 | 76 |
| (17) | 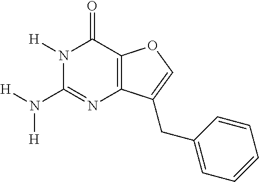 | 0 |
| (29) | 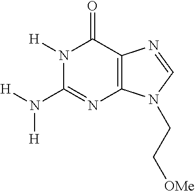 | >400 |
| (31) | 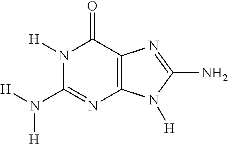 | 27 |
| (32) | 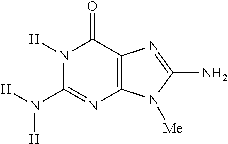 | 0 |
| (37) | 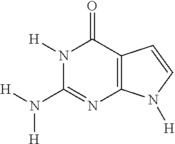 | 43 |
| (38) | 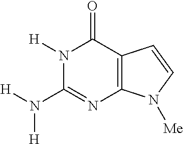 | 193 |
| (40) | 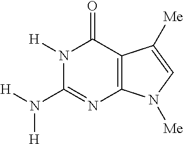 | >400 |
| (52) | 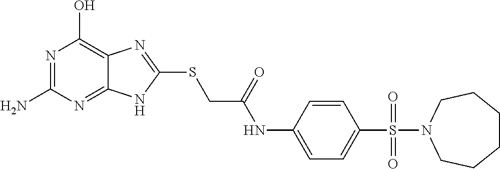 | 127 |
| (54) | 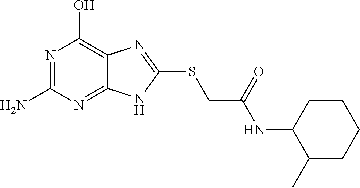 | 89 |
| (56) | 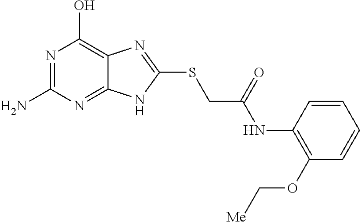 | 40 |

### Other Embodiments

While the invention has been described in connection with specific embodiments thereof, it will be understood that it is capable of further modifications and this application is intended to cover any variations, uses, or adaptations of the invention following, in general, the principles of the invention and including such departures from the present disclosure that come within known or customary practice within the art to which the invention pertains and may be applied to the essential features hereinbefore set forth.

All references, patents, patent application publications, and patent applications cited herein are hereby incorporated by reference to the same extent as if each of these references, patents, patent application publications, and patent applications were separately incorporated by reference herein.

What is claimed is:

1. A compound having a structure according to Formula (I): 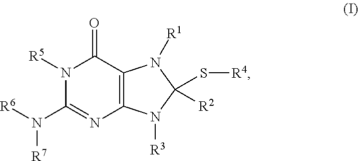
or a tautomer, prodrug, or pharmaceutically acceptable salt thereof, wherein:
R¹, R², R³, and R⁴ are each, independently, H, optionally substituted C₁₋₆ alkyl, or R¹ and R², R² and R³, or R² and R⁴ combine to form a double bond,
R⁵, R⁶, and R⁷ are each, independently, H or optionally substituted C₁₋₆ alkyl, and
wherein one and only one of R¹ and R², R² and R³, or R² and R⁴ combine to form a double bond, and
when R⁵, R⁶, and R⁷ are H, R¹ and R² combine to form a double bond and R³ is H, or when R⁵, R⁶, and R⁷ are H, R² and R³ combine to form a double bond and R¹ is H, R⁴ is not —CH₂C₆H₅, —CH₂(p-C₆H₄—CN), —CH₂(p-C₆H₄—CH₃), —CH₂CH═CH₂, —CH₂C(═O)-(p-C₆H₄-OMe), —CH₂C(═O)NH-(o-C₆H₄-OEt), —CH₂C(═O)NH-(2-methoxy-5-chloro-C₆H₃), —CH₂C(═O)NH-(2-methylcyclohexyl), or —CH₂C(═O)NH-(p-C₆H₄—SO₂(azepane)).

2. The compound of claim 1, wherein R⁵, R⁶, and R⁷ are each H.

3. The compound of claim 1, wherein R⁶ is optionally substituted C₁₋₆ alkyl.

4. The compound of claim 1 or 2, wherein R¹ and R² combine to form a double bond.

5. The compound of claim 4, wherein R³ is H.

6. The compound of claim 1 or 2, wherein R² and R³ combine to form a double bond.

7. The compound of claim 6, wherein R¹ is H.

8. The compound of any of claims 1-7, wherein R⁴ is optionally substituted C₁₋₆ alkyl.

9. The compound of claim 8, wherein said C₁₋₆ alkyl group comprises a substituent selected from aryl, heteroaryl, cycloalkyl, heterocyclyl, alkenyl, hydroxyl, C₁₋₃ alkoxy, amino, or C₁₋₆ alkylamino, and wherein said aryl or heteroaryl is optionally substituted by C₁₋₄ alkyl, halogen, or nitrile.

10. The compound of claim 1, wherein said compound has a structure according to one of the following formulas:

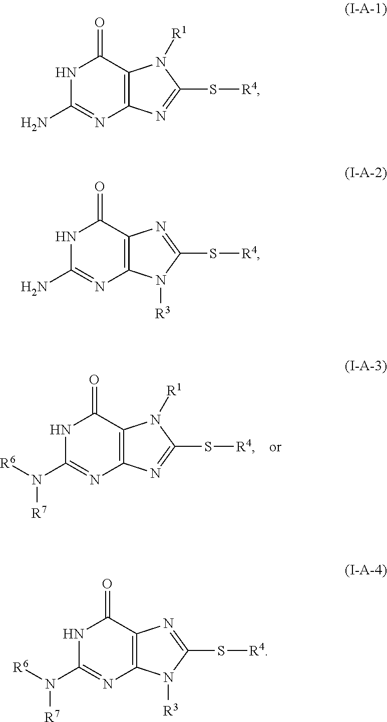

11. The compound of claim 10, or a tautomer, prodrug, or pharmaceutically acceptable salt thereof, wherein said compound is selected from the group consisting of:

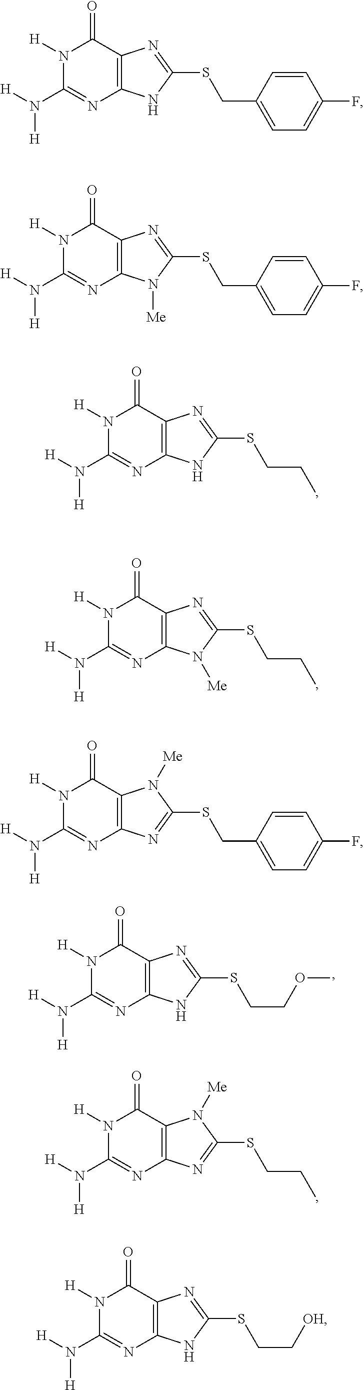

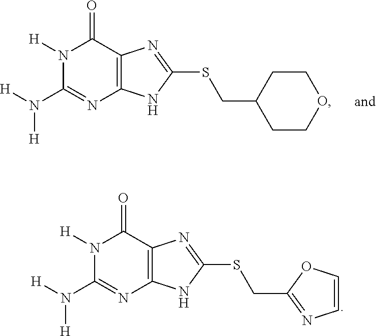

12. The compound of claim 1 or 2, wherein R² and R⁴ combine to form a double bond.

13. The compound of claim 1 or 2, wherein said compound has a structure according to one of the following formulas:

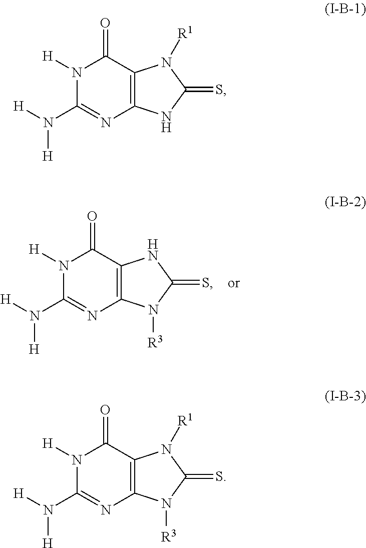

14. The compound of claim 13, wherein said compound is selected from the group consisting of:

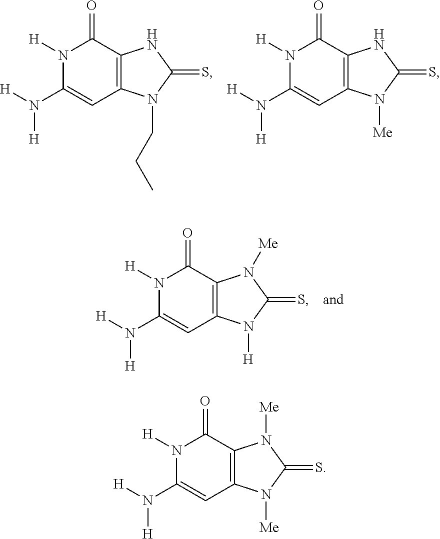

15. A compound having a structure according to Formula (III): 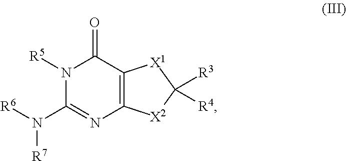
or a tautomer, prodrug, or pharmaceutically acceptable salt thereof, wherein
X¹ is O or NR¹;
X² is O or NR²;
R¹ and R² are each, independently, selected from H, or optionally substituted C₁₋₆ alkyl;
R³ is H, halogen, or NR⁸R⁹, or R³ combines with R⁴ to form an oxo group; and
R⁴ combines with R¹ or R² to form a C═N bond or R⁴ combines with R³ to form an oxo group;
R⁵, R⁶, R⁷, R⁸, and R⁹ are each, independently, H or optionally substituted C₁₋₆ alkyl; and
when R⁵, R⁶, and R⁷ are H, X¹ is NR¹, R¹ and R⁴ combine to form a C═N double bond, and X² is NH, R³ is not H or NH₂, and
when R⁵, R⁶, and R⁷ are H, X¹ is NH, R³ combines with R⁴ to form an oxo group, and X² is NR², R² is not H.

16. The compound of claim 15, wherein R⁵, R⁶, R⁷, R⁸, and R⁹ are each H.

17. The compound of claim 15 or 16, wherein said C₁₋₆ alkyl group comprises a substituent selected from aryl, heteroaryl, cycloalkyl, heterocyclyl, hydroxyl, C₁₋₃ alkoxy, amino, or C₁₋₆alkylamino, and wherein said aryl or heteroaryl is optionally substituted by C₁₋₄ alkyl, halogen, or nitrile.

18. The compound of any of claims 15-17, wherein said compound has the following structure:

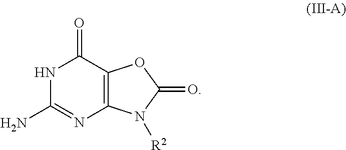

19. The compound of claim 18, wherein said compound is selected from the group consisting of:

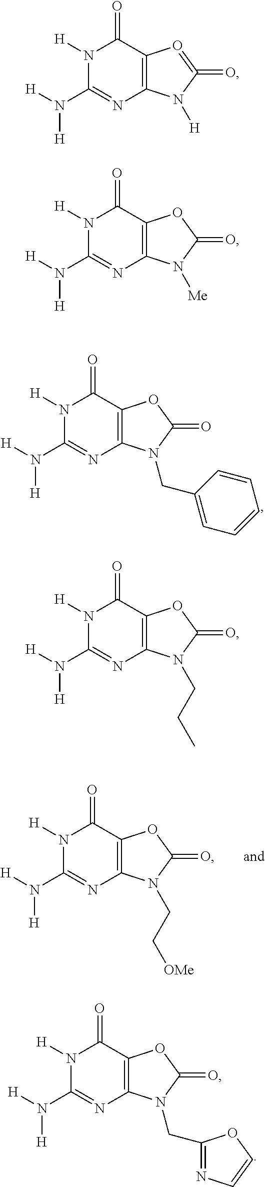

20. The compound of any of claims 15-17, wherein X¹ is NR¹, X² is NR², R¹ and R² are each, independently, H or optionally substituted C₁₋₆ alkyl, and R³ combines with R⁴ to form an oxo group.

21. The compound of claim 20, wherein said compound is

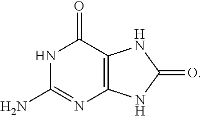

22. The compound of any of claims 15-17, wherein said compound has a structure according to

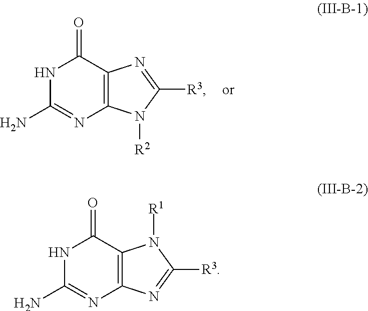

23. The compound of claim 22, wherein R³ is H.

24. The compound of claim 22 or 23, wherein said compound has a structure according to 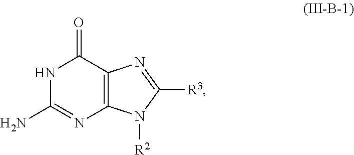
and wherein R² is optionally substituted C₁₋₆ alkyl.

25. The compound of claim 24, wherein said C₁₋₆ alkyl group comprises a substituent selected from aryl, heteroaryl, cycloalkyl, heterocyclyl, hydroxyl, C₁₋₃ alkoxy, amino, or C₁₋₆alkylamino, and wherein said aryl or heteroaryl is optionally substituted by C₁₋₄ alkyl, halogen, or nitrile.

26. The compound of claim 24, wherein R³ is Cl or Br.

27. The compound of claim 22, wherein said compound is selected from the group consisting of:

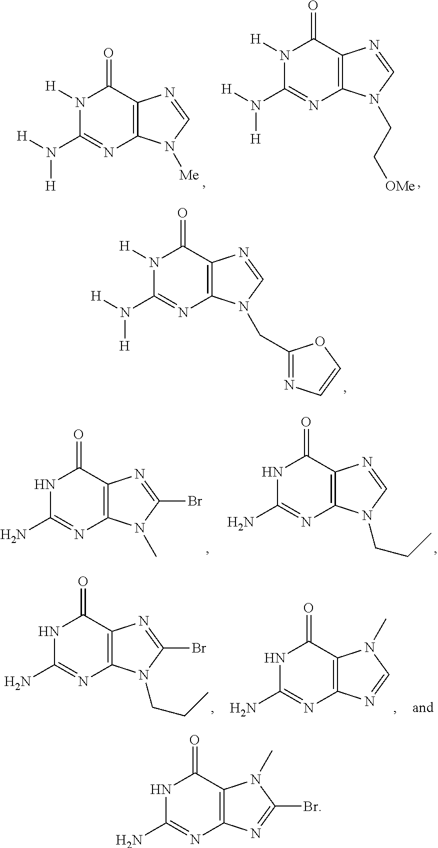

28. The compound of any of claims 15-17, wherein said compound has a structure according to the following formula:

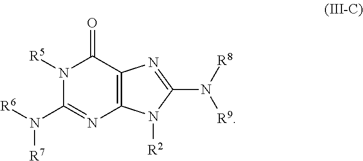

29. The compound of claim 28, wherein R⁵, R⁶, R⁷, R⁸, and R⁹ are each H, and R² is optionally substituted C₁₋₆ alkyl.

30. The compound of claim 28, wherein R² is H.

31. The compound of any of claims 28-30, wherein said C₁₋₆ alkyl group comprises a substituent selected from aryl, heteroaryl, cycloalkyl, heterocyclyl, hydroxyl, C₁₋₃ alkoxy, amino, or C₁₋₆alkylamino, and wherein said aryl or heteroaryl is optionally substituted by C₁₋₄ alkyl, halogen, or nitrile.

32. The compound of claim 29, wherein said compound is

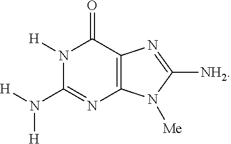

33. A compound having a structure according to 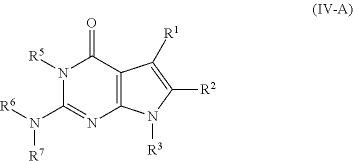
or a tautomer, prodrug, or pharmaceutically acceptable salt thereof, or according to 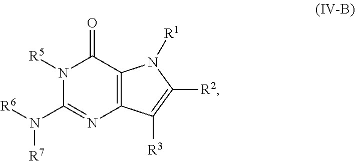
or a tautomer, prodrug, or pharmaceutically acceptable salt thereof, wherein R¹, R², R³, R⁵, R⁶, and R⁷ are each, independently, H or optionally substituted C₁₋₆ alkyl.

34. The compound of claim 33, wherein R⁵, R⁶, and R⁷ are each H.

35. The compound of claim 33 or 34, wherein R¹ and R³ are both H.

36. The compound of any of claims 33-35, wherein said C₁₋₆ alkyl group comprises a substituent selected from aryl, heteroaryl, cycloalkyl, heterocyclyl, hydroxyl, C₁₋₃ alkoxy, amino, or C₁₋₆alkylamino, and wherein said aryl or heteroaryl is optionally substituted by C₁₋₄ alkyl, halogen, or nitrile.

37. The compound of claim 36, wherein R² is H.

38. The compound of claim 37, wherein said compound is selected from the group consisting of:

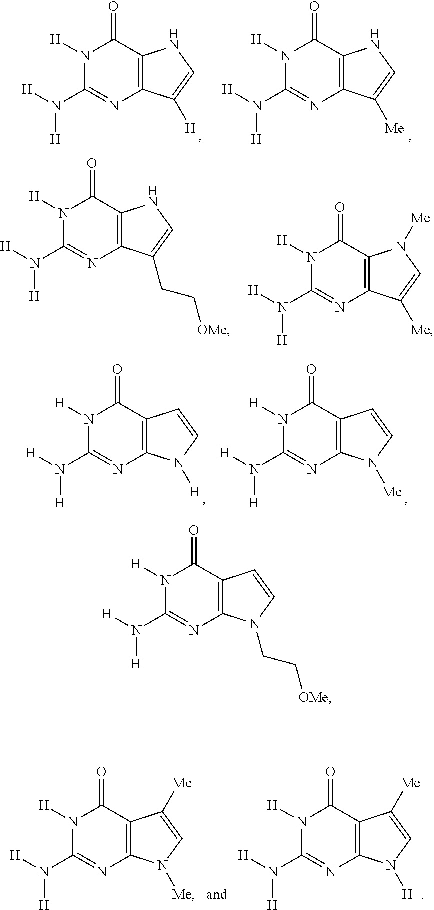

39. A compound selected from the group consisting of: 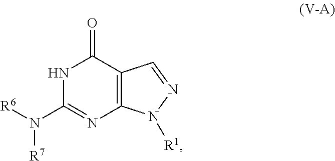
or a tautomer, prodrug, or pharmaceutically acceptable salt thereof,
and 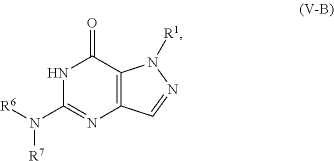
or a tautomer, prodrug, or pharmaceutically acceptable salt thereof, wherein each of R¹, R⁶, and R⁷, is H or optionally substituted C₁₋₆ alkyl.

40. The compound of claim 39, wherein R⁶ and R⁷ are both H.

41. The compound of claim 39, wherein said C₁₋₆ alkyl group comprises a substituent selected from aryl, heteroaryl, cycloalkyl, heterocyclyl, hydroxyl, C₁₋₃ alkoxy, amino, or C₁₋₆alkylamino, and wherein said aryl or heteroaryl is optionally substituted by C₁₋₄ alkyl, halogen, or nitrile.

42. The compound of claim 41, wherein R¹ is H.

43. The compound of claim 39, wherein said compound is 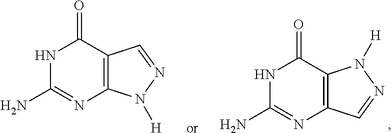
or a tautomer, prodrug, or pharmaceutically acceptable salt thereof.

44. A compound according to Formula (VI), 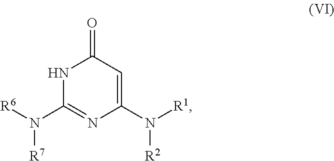
or a tautomer, prodrug, or pharmaceutically acceptable salt thereof, wherein each of R¹, R², R⁶, and R⁷ is, independently, H or optionally substituted C₁₋₆ alkyl.

45. The compound of claim 44, wherein R⁶ and R⁷ are both H.

46. The compound of claim 44, wherein said compound is

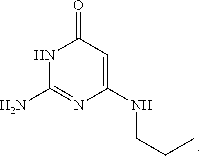

47. The compound of any of claims 1-46, wherein said compound is an inhibitor of GTP cyclohydrolase (GCH-1).

48. A pharmaceutical composition comprising the compound of any of claims 1-47, or any of the following compounds, 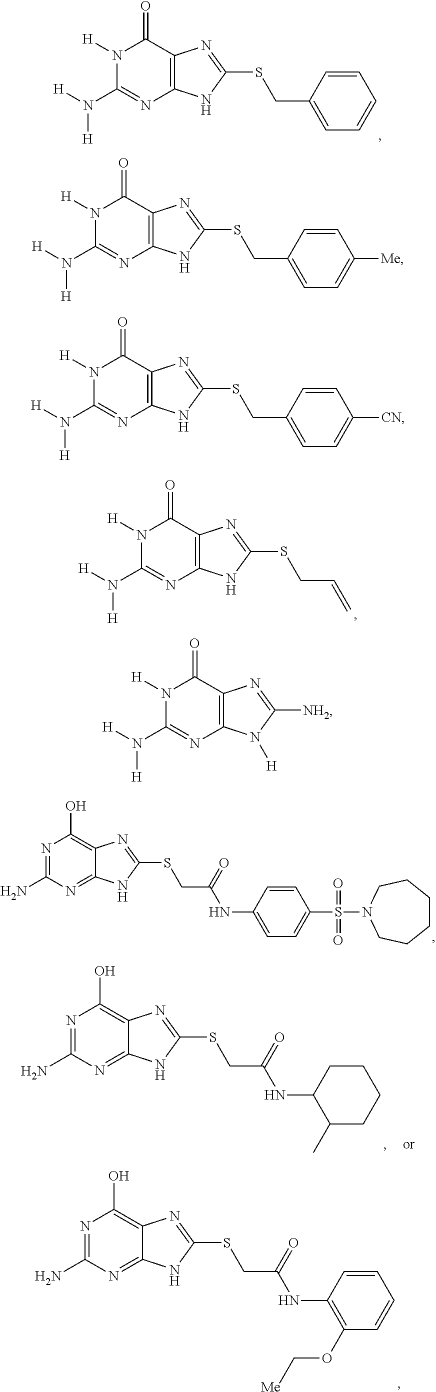
or a tautomer, prodrug, or pharmaceutically acceptable salt thereof, and a pharmaceutically acceptable excipient.

49. A method of treating, reducing, or preventing a condition in a mammal, wherein said method comprises the administration of the compound of any of claims 1-47, or any of the following compounds, 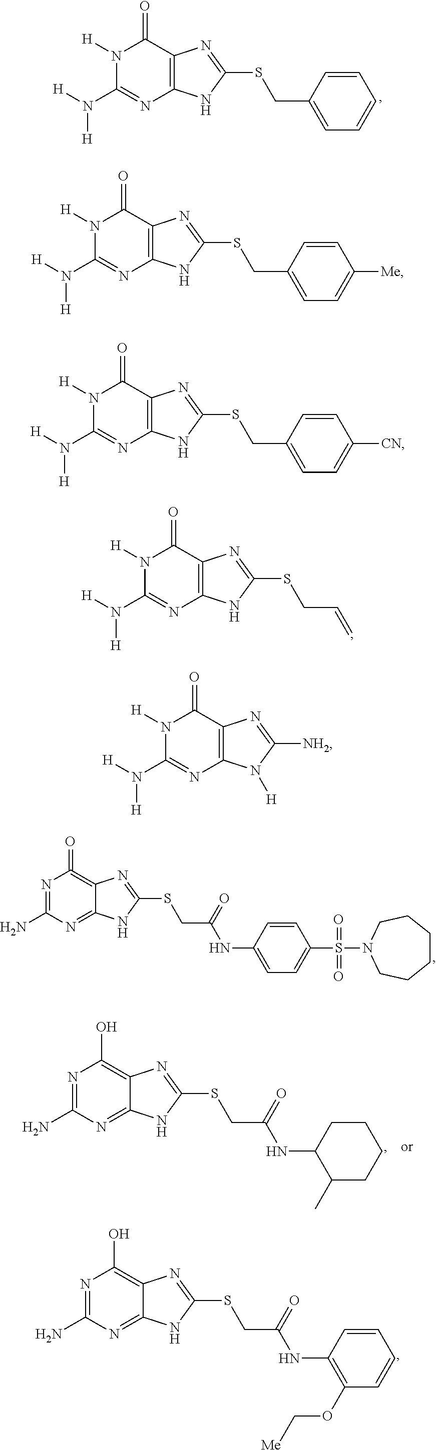
or a tautomer, prodrug, or pharmaceutically acceptable salt thereof, or the pharmaceutical composition of claim 48, to said mammal in a dosage sufficient to inhibit GCH-1.

50. The method of claim 49, wherein said condition is pain.

51. The method of claim 50, wherein said pain is neuropathic, inflammatory, nociceptive, or functional pain.

52. The method of claim 50 or 51, wherein said pain is chronic pain.

53. The method of claim 50 or 51, wherein said pain is acute pain.

54. A method of inhibiting GCH-1 in a cell, wherein said method comprises contacting a cell with any of the compounds of claims 1-47, or any of the following compounds, 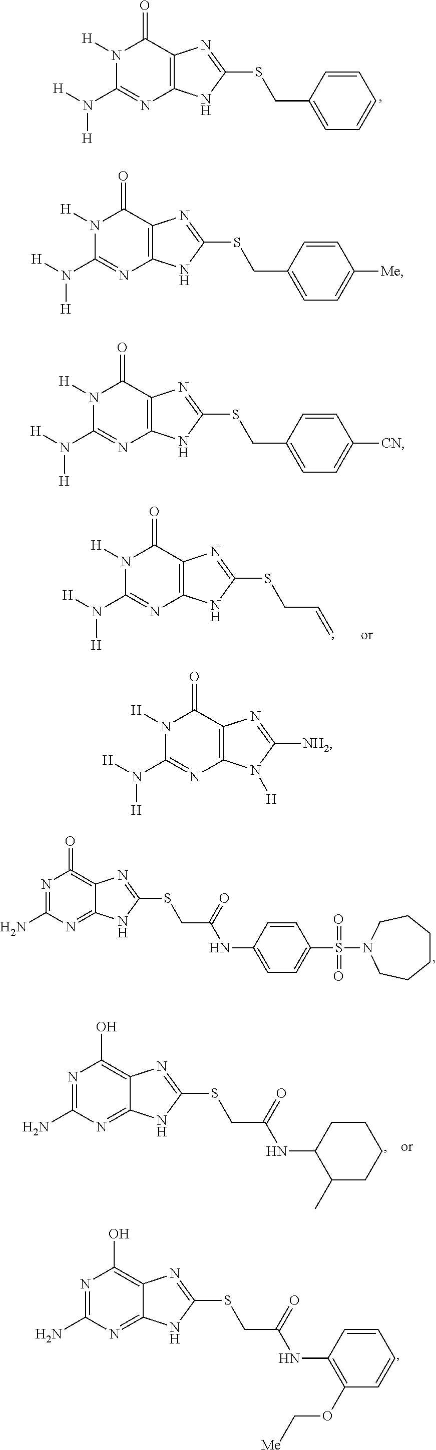
or a tautomer, prodrug, or pharmaceutically acceptable salt thereof.
